SEQUENCE LISTING (89 sequences)

SEQ ID NO: 1 - protein, 13 aa, Artificial sequence
  synthetic sequence
FVQWFSKFLGRIL

SEQ ID NO: 2 - protein, 13 aa, Artificial sequence
  synthetic sequence
FVQWFSKFLGKLL

SEQ ID NO: 3 - protein, 11 aa, Artificial sequence
  synthetic sequence
FVQWFSKFLGK

SEQ ID NO: 4 - protein, 11 aa, Artificial sequence
  synthetic sequence
FFQWFSKFLGK

SEQ ID NO: 5 - protein, 12 aa, Artificial sequence
  synthetic sequence
ILGTILGLLKGL

SEQ ID NO: 6 - protein, 15 aa, Artificial sequence
  synthetic sequence
  VARIANT (5)..(5): The amino acid at position 5 is Arg or Gln
SFLLXNPNDKYEPFW

SEQ ID NO: 7 - protein, 10 aa, Artificial sequence
  synthetic sequence
QDPNDQYEPF

SEQ ID NO: 8 - protein, 13 aa, Artificial sequence
  synthetic sequence
  VARIANT (4)..(4): The amino acid at position 4 is Ile or Leu
  VARIANT (10)..(10): The amino acid at position 10 is Ser or Gly
FLPXIASLLXKLL

SEQ ID NO: 9 - protein, 13 aa, Artificial sequence
  synthetic sequence
FLPLIGRVLSGIL

SEQ ID NO: 10 - protein, 13 aa, Artificial sequence
  synthetic sequence
LLPIVGNLLKSLL

SEQ ID NO: 11 - protein, 13 aa, Artificial sequence
  synthetic sequence
LLPILGNLLNGLL

SEQ ID NO: 12 - protein, 13 aa, Artificial sequence
  synthetic sequence
LLPIVGNLLNSLL

SEQ ID NO: 13 - protein, 13 aa, Artificial sequence
  synthetic sequence
VLPIIGNLLNSLL

SEQ ID NO: 14 - protein, 13 aa, Artificial sequence
  synthetic sequence
FLPLIGKVLSGIL

SEQ ID NO: 15 - protein, 13 aa, Artificial sequence
  synthetic sequence
FFPVIGRILNGIL

SEQ ID NO: 16 - protein, 10 aa, Artificial sequence
  synthetic sequence
LSPNLLKSLL

SEQ ID NO: 17 - protein, 10 aa, Artificial sequence
  synthetic sequence
LLPNLLKSLL

SEQ ID NO: 18 - protein, 13 aa, Artificial sequence
  synthetic sequence
FLPFLAKILTGVL

SEQ ID NO: 19 - protein, 13 aa, Artificial sequence
  synthetic sequence
FLPLFASLIGKLL

SEQ ID NO: 20 - protein, 13 aa, Artificial sequence
  synthetic sequence
FLPFLASLLTKVL

SEQ ID NO: 21 - protein, 13 aa, Artificial sequence
  synthetic sequence
FLPFLASLLSKVL

SEQ ID NO: 22 - protein, 13 aa, Artificial sequence
  synthetic sequence
FLPFLATLLSKVL

SEQ ID NO: 23 - protein, 14 aa, Artificial sequence
  synthetic sequence
SILPTIVSFLSKVF

SEQ ID NO: 24 - protein, 14 aa, Artificial sequence
  synthetic sequence
SILPTIVSFLSKFL

SEQ ID NO: 25 - protein, 14 aa, Artificial sequence
  synthetic sequence
SILPTIVSFLTKFL

SEQ ID NO: 26 - protein, 14 aa, Artificial sequence
  synthetic sequence
FILPLIASFLSKFL

SEQ ID NO: 27 - protein, 13 aa, Artificial sequence
  synthetic sequence
VLPLISMALGKLL

SEQ ID NO: 28 - protein, 14 aa, Artificial sequence
  synthetic sequence
NFLGTLINLAKKIM

SEQ ID NO: 29 - protein, 14 aa, Artificial sequence
  synthetic sequence
FLPILINLIHKGLL

SEQ ID NO: 30 - protein, 13 aa, Artificial sequence
  synthetic sequence
FLPIVGKLLSGLL

SEQ ID NO: 31 - protein, 12 aa, Artificial sequence
  synthetic sequence
FLPIASLLGKYL

SEQ ID NO: 32 - protein, 13 aa, Artificial sequence
  synthetic sequence
FISAIASMLGKFL

SEQ ID NO: 33 - protein, 13 aa, Artificial sequence
  synthetic sequence
FLSAIASMLGKFL

SEQ ID NO: 34 - protein, 13 aa, Artificial sequence
  synthetic sequence
FISAIASFLGKFL

SEQ ID NO: 35 - protein, 14 aa, Artificial sequence
  synthetic sequence
FLFPLITSFLSKVL

SEQ ID NO: 36 - protein, 13 aa, Artificial sequence
  synthetic sequence
FLPAIAGILSQLF

SEQ ID NO: 37 - protein, 13 aa, Artificial sequence
  synthetic sequence
FLPLIAGLLGKLF

SEQ ID NO: 38 - protein, 14 aa, Artificial sequence
  synthetic sequence
FFPIGVFCKIFKTC

SEQ ID NO: 39 - protein, 14 aa, Artificial sequence
  synthetic sequence
FFPLALLCKVFKKC

SEQ ID NO: 40 - protein, 14 aa, Artificial sequence
  synthetic sequence
FLLFPLMCKIQGKC

SEQ ID NO: 41 - protein, 14 aa, Artificial sequence
  synthetic sequence
FVLPLVMCKILRKC

SEQ ID NO: 42 - protein, 21 aa, Artificial sequence
  synthetic sequence
FGLPMLSILPKALCILLKRKC

SEQ ID NO: 43 - protein, 6 aa, Artificial sequence
  synthetic sequence
RRWWRF

SEQ ID NO: 44 - protein, 6 aa, Artificial sequence
  synthetic sequence
FRWWHR

SEQ ID NO: 45 - protein, 8 aa, Artificial sequence
  synthetic sequence
PFKLSLHL

SEQ ID NO: 46 - protein, 9 aa, Artificial sequence
  synthetic sequence
TPFKLSLHL

SEQ ID NO: 47 - protein, 8 aa, Artificial sequence
  synthetic sequence
FFFLSRIF

SEQ ID NO: 48 - protein, 8 aa, Artificial sequence
  synthetic sequence
FFWLSKIF

SEQ ID NO: 49 - protein, 7 aa, Artificial sequence
  synthetic sequence
KVFLGLK

SEQ ID NO: 50 - protein, 12 aa, Artificial sequence
  synthetic sequence
GIHDILKYGKPS

SEQ ID NO: 51 - protein, 13 aa, Artificial sequence
  synthetic sequence
ILGKIWEGIKSLF

SEQ ID NO: 52 - protein, 14 aa, Artificial sequence
  synthetic sequence
LKLKSIVSWAKKVL

SEQ ID NO: 53 - protein, 14 aa, Artificial sequence
  synthetic sequence
KKKKPLFGLFFGLF

SEQ ID NO: 54 - protein, 14 aa, Artificial sequence
  synthetic sequence
INWLKLGKAIIDAL

SEQ ID NO: 55 - protein, 15 aa, Artificial sequence
  synthetic sequence
SFLLRNPNDKYEPFW

SEQ ID NO: 56 - protein, 15 aa, Artificial sequence
  synthetic sequence
SFLLQDPNDQYEPFW

SEQ ID NO: 57 - protein, 8 aa, Artificial sequence
  synthetic sequence
IEPDVSQV

SEQ ID NO: 58 - protein, 8 aa, Artificial sequence
  synthetic sequence
WAFRSRYH

SEQ ID NO: 59 - protein, 8 aa, Artificial sequence
  synthetic sequence
ASPRAGGK

SEQ ID NO: 60 - protein, 8 aa, Artificial sequence
  synthetic sequence
KEPLSAEA

SEQ ID NO: 61 - protein, 8 aa, Artificial sequence
  synthetic sequence
  SITE (4)..(4): The amino acid at position 4 is D-aspartic acid
IEPDVSQV

SEQ ID NO: 62 - protein, 10 aa, Artificial sequence
  synthetic sequence
  SITE (1)..(2): The amino acids at positions 1 and 2 comprise 7-methoxycoumarin-4-acetic acid (MCA)
  SITE (10)..(10): The amino acid at position 10 comprises a DNP quencher
KIEPDVSQVK

SEQ ID NO: 63 - protein, 29 aa, Artificial sequence
  synthetic sequence
  SITE (1)..(1): The amino acid at position 1 comprises dota-hexanoic acid
FVQWFSKFLGKIEPDVSQVQDPNDQYEPF

SEQ ID NO: 64 - protein, 8 aa, Artificial sequence
  synthetic sequence
IEPDVSVQ

SEQ ID NO: 65 - protein, 8 aa, Artificial sequence
  synthetic sequence
LTYDFWIQ

SEQ ID NO: 66 - protein, 8 aa, Artificial sequence
  synthetic sequence
PQVDLYDK

SEQ ID NO: 67 - protein, 8 aa, Artificial sequence
  synthetic sequence
VVQDKHEI

SEQ ID NO: 68 - protein, 8 aa, Artificial sequence
  synthetic sequence
VYADSSEW

SEQ ID NO: 69 - protein, 8 aa, Artificial sequence
  synthetic sequence
TMADSQES

SEQ ID NO: 70 - protein, 8 aa, Artificial sequence
  synthetic sequence
  VARIANT (7)..(8): Xaa can be any naturally occurring amino acid
GHIDHMXX

SEQ ID NO: 71 - protein, 8 aa, Artificial sequence
  synthetic sequence
LEQDVWIA

SEQ ID NO: 72 - protein, 8 aa, Artificial sequence
  synthetic sequence
LDPDNFKR

SEQ ID NO: 73 - protein, 8 aa, Artificial sequence
  synthetic sequence
  VARIANT (1)..(2): Xaa can be any naturally occurring amino acid
XXPDFYLG

SEQ ID NO: 74 - protein, 8 aa, Artificial sequence
  synthetic sequence
MGPDAFNL

SEQ ID NO: 75 - protein, 8 aa, Artificial sequence
  synthetic sequence
  VARIANT (7)..(8): Xaa can be any naturally occurring amino acid
LKDDMGXX

SEQ ID NO: 76 - protein, 8 aa, Artificial sequence
  synthetic sequence
IWFDYTLK

SEQ ID NO: 77 - protein, 8 aa, Artificial sequence
  synthetic sequence
  VARIANT (1)..(1): Xaa can be any naturally occurring amino acid
XIGDNVEW

SEQ ID NO: 78 - protein, 8 aa, Artificial sequence
  synthetic sequence
  VARIANT (1)..(3): Xaa can be any naturally occurring amino acid
XXXDQVNL

SEQ ID NO: 79 - protein, 8 aa, Artificial sequence
  synthetic sequence
  VARIANT (7)..(8): Xaa can be any naturally occurring amino acid
PQADQWXX

SEQ ID NO: 80 - protein, 8 aa, Artificial sequence
  synthetic sequence
  VARIANT (6)..(8): Xaa can be any naturally occurring amino acid
PSVDMXXX

SEQ ID NO: 81 - protein, 8 aa, Artificial sequence
  synthetic sequence
  VARIANT (1)..(1): Xaa can be any naturally occurring amino acid
XNVDWTAP

SEQ ID NO: 82 - protein, 8 aa, Artificial sequence
  synthetic sequence
YGYDLQTA

SEQ ID NO: 83 - protein, 8 aa, Artificial sequence
  synthetic sequence
HGFDEAHN

SEQ ID NO: 84 - protein, 8 aa, Artificial sequence
  synthetic sequence
HSHDSWKA

SEQ ID NO: 85 - protein, 8 aa, Artificial sequence
  synthetic sequence
KQDDLMSE

SEQ ID NO: 86 - protein, 8 aa, Artificial sequence
  synthetic sequence
SFGDIMEM

SEQ ID NO: 87 - protein, 8 aa, Artificial sequence
  synthetic sequence
  VARIANT (7)..(8): Xaa can be any naturally occurring amino acid
VNDDVKXX

SEQ ID NO: 88 - protein, 8 aa, Artificial sequence
  synthetic sequence
  VARIANT (1)..(3): Xaa can be any naturally occurring amino acid
XXXDKQFT

SEQ ID NO: 89 - protein, 8 aa, Artificial sequence
  synthetic sequence
  VARIANT (7)..(8): Xaa can be any naturally occurring amino acid
NDVDGGXX

(19) United States
(12) Patent Application Publication
(10) Pub. No.: US 2024/0238459 A1
(43) Pub. Date: Jul. 18, 2024

(54) Granzyme-Activatable Membrane-Interacting Peptides and Methods of Use

(71) Applicant: The Regents of the University of California, Oakland, CA (US)

(72) Inventors: Charles S. Craik, San Francisco, CA (US); Conner Bardine, San Francisco, CA (US); Michael Evans, San Francisco, CA (US)

(21) Appl. No.: 18/563,182

(22) PCT Filed: Jun. 30, 2022
(86) PCT No.: PCT/US2022/035779

§ 371 Date: Nov. 21, 2023

Related U.S. Application Data
(60) Provisional application No. 63/216,890, filed on Jun. 30, 2021.

(51) Int. Cl. A61K 51/08; A61K 47/64; A61K 47/65; A61K 49/00; C12Q 1/37

(52) U.S. Cl. CPC A61K 51/088; A61K 47/64; A61K 47/65; A61K 49/0032; A61K 49/0056; C12Q 1/37

(57) ABSTRACT

The present disclosure provides granzyme-activatable and detectable membrane-interacting peptides that, following activation, can interact with phospholipid bilayers, such as cell membranes. The present disclosure also provides methods of use of such peptides, as well as compositions comprising such peptides. The peptides of the present disclosure are of the general structure X^{1a}-A-X²—Z—X^{1b}, where A is a membrane-interacting peptide region having a plurality of nonpolar hydrophobic amino acid residues that, following separation from portions Z, is capable of interaction with a phospholipid bilayer; Z is an inhibitory peptide region that can inhibit the activity of portion A; X² is a granzyme-cleavable linker that can be cleaved to release cleavage products from the compound; and X^{1a} and X^{1b} are optionally-present chemical handles that facilitate conjugation of various cargo moieties to the compound.

### CROSS-REFERENCE TO RELATED APPLICATIONS

This application claims the benefit of U.S. Provisional Patent Application No. 63/216,890, filed Jun. 30, 2021, which application is incorporated herein by reference in its entirety.

### STATEMENT OF GOVERNMENT SUPPORT

This invention was made with government support under grants R01 EB025207, R01 CA258297, and R01 AI161027 awarded by The National Institutes of Health. The government has certain rights in the invention.

### INTRODUCTION

The human granzymes consist of five serine proteases (A, B, H, K, M) that are expressed primarily within the secretory vesicles (i.e., granules) of lymphocytes involved in host defense, namely natural killer (NK) and cytotoxic T cells (CTLs). In these cell types, granzymes are best understood to be pro-apoptotic effectors against problematic cells, for example cancer cells or cells infected with pathogens. To impart cytotoxicity, lymphocytes degranulate after docking with a target cell to release granzymes transiently into pericellular space. Co-secreted with granzymes are perforin molecules, which form a channel in the plasma membrane of the target cell to facilitate granzyme transit into the cytoplasm. Granzyme biochemistry subsequently triggers cell death through several mechanisms, for example proteolytic activation of caspases or direct DNA damage (granzyme B), and SET mediated activation of DNA cleavage (granzyme A). However, the canon that granzymes are primarily cytotoxic effectors is being challenged by a more complex biological model in which secreted granzymes can also persist in extracellular space to perform non-cytotoxic signaling functions.

### SUMMARY

The present disclosure generally provides granzyme-activatable and detectable membrane-interacting peptides that, following activation, can interact with phospholipid bilayers, such as cell membranes. The present disclosure also provides methods of use of such compounds.

The compounds of the present disclosure are of the general structure X^{1a}-A-X²—Z—X^{1b}, where A is a membrane-interacting peptide region having a plurality of nonpolar hydrophobic amino acid residues that, following separation from portion Z, is capable of interacting with a phospholipid bilayer; Z is an inhibitory peptide region that can inhibit the activity of portion A; X² is a granzyme-cleavable linker that can be cleaved to release cleavage products from the compound; and X^{1a} and X^{1b} are optionally-present chemical handles that facilitate conjugation of various moieties to the compound. Prior to cleavage of the composition at X², the composition acts as a promolecule that does not associate with phospholipid bilayers to a significant or detectable level. Following cleavage at cleavable linker X², the cleavage product including portion A is free to interact with a phospholipid bilayer (e.g., a cell membrane), and thus accumulate at a site associated with a cleavage-promoting environment. Detection of the membrane-associated cleavage product can be accomplished by detection of a moiety attached through X^{1a} and/or X^{1b}. Such compositions can be used in a variety of methods, including, for example, use in directly imaging granzyme activity within a subject.

In some embodiments, the present disclosure provides molecules that include the structure, from N-terminal to C-terminal or C-terminal to N-terminal: X^{1a}-A-X²—Z—X^{1b}. wherein X^{1a} and/or X^{1b} may be present or absent, and when present comprise a nucleophilic moiety; A is a membrane-interacting polypeptide portion that, when separated from portion Z, comprises an alpha-helical structure capable of inserting into a phospholipid bilayer; Z is a polypeptide that, when linked to portion A through portion X², is effective to inhibit interaction of portion A with a phospholipid bilayer; and X² is a cleavable linker, wherein X² joins portion A to portion Z, and wherein X² can be cleaved under physiological conditions. In some embodiments, portion A includes about 5 to about 30 amino acid residues. In some embodiments, portion A includes the amino acid sequence X^{a}X^{b}X^{c}X^{d}X^{e}X^{f}Y^{a}X^{g}X^{h}Y^{b}Y*XⁱX^{j}, where X^{a}, X^{b}, X^{c}, X^{d}, X^{e}, X^{f}, X^{g}, X^{h}, Xⁱ, and X^{j} are hydrophobic amino acid residues, Y^{a} and Y^{b} are hydrophilic amino acid residues, and Y* is a charged amino acid residue. In some embodiments, portion A includes the amino acid sequence FVQWFSKFLGRIL (SEQ ID NO:1), or a conservative amino acid substitution thereof. In some embodiments, portion A includes the amino acid sequence FVQWFSKFLGKLL (SEQ ID NO:2), or a conservative amino acid substitution thereof. In some embodiments, portion A includes the amino acid sequence FVQWFSKFLGK (SEQ ID NO:3), or a conservative amino acid substitution thereof. In some embodiments, portion A includes the amino acid sequence FFQWFSKFLGK (SEQ ID NO:4), or a conservative amino acid substitution thereof. In some embodiments, portion A includes the amino acid sequence ILGTILGLLKGL (SEQ ID NO:5). In some embodiments, portion A includes the amino acid sequence of Japonicin-1. In some embodiments, portion A includes fewer than 5 basic amino acid residues.

In some embodiments, X² is cleavable by a granzyme. In some embodiments, X² is cleavable by granzyme B. In some embodiments, X² is cleavable by granzyme K. In some embodiments, X² is an enzymatically cleavable linker and Z includes an exosite recognition sequence for a granzyme that is capable of cleaving X².

In some embodiments, portion Z includes a covalently linked water soluble polymer. In some embodiments, Z includes the amino acid sequence SFLL(X^{a})NPNDKYEPFW (SEQ ID NO:6), wherein X^{a} is R or Q. In some embodiments, Z includes the amino acid sequence QDPNDQYEPF (SEQ ID NO:7). In some embodiments, Z comprises an amino acid sequence of an exosite recognition sequence for granzyme.

In some embodiments, one or more of X^{1a}, X^{1b}, A, or Z includes a D-amino acid. In some embodiments, X^{1a} is present and includes a nucleophilic moiety. In some embodiments, X^{1b} is present and includes a nucleophilic moiety. In some embodiments, the nucleophilic moiety of X^{1a} or X^{1b} includes a thiol functional group. In some embodiments, X^{1a} or X^{1b} includes an amino acid residue that includes the nucleophilic moiety. In some embodiments, the amino acid residue is a cysteine residue. In some embodiments, the amino acid residue is a lysine residue.

In some embodiments, X^{1a} or X^{1b} includes a cargo moiety covalently attached to the nucleophilic moiety. In some embodiments, the cargo moiety is a detectable moiety. In some embodiments, the detectable moiety includes a fluorescent moiety. In some embodiments, the detectable moiety comprises a radioisotope. In some embodiments, the present disclosure provides nucleic acids encoding the molecule described above. In some embodiments, the present disclosure provides compositions that include the molecules described above and a pharmaceutically acceptable carrier.

In some embodiments, the present disclosure provides methods of detectably labeling a phospholipid bilayer in the presence of granzyme activity, the methods including contacting a molecule as described above with a granzyme contributing to the granzyme activity, wherein when the contacting is under conditions suitable for granzyme cleavage of the cleavable linker, the molecule is cleaved to release the membrane interacting polypeptide portion for interaction with a phospholipid bilayer and detectably labels the phospholipid bilayer. In some embodiments, the cell is in vivo. In some embodiments, the subject is a human.

In some embodiments, the present disclosure provides methods for assessing granzyme activity in a subject, the methods including administering to the subject a molecule as described above, wherein X² is cleavable by a granzyme, wherein in the presence of granzyme activity the molecule is cleaved to release a cleavage product comprising the detectable moiety and the membrane interacting polypeptide portion and wherein the cleavage product interacts with a phospholipid bilayer in an area of granzyme enzyme activity, and detecting the presence or absence of the detectable label of the cleavage product, wherein the presence of the detectable label indicates an area of granzyme enzyme activity. In some embodiments, the assessing can be qualitative or and/or quantitative.

In some embodiments, the present disclosure provides methods for assessing immune cell activation in a subject, wherein the immune cells secrete granzyme upon activation, the methods including administering to the subject a molecule as described above, wherein X² is cleavable by a granzyme, wherein in the presence of activated granzyme-secreting immune cells the molecule is cleaved to release a cleavage product comprising the detectable moiety and the membrane interacting polypeptide portion and wherein the cleavage product interacts with a phospholipid bilayer in an area of granzyme-secreting immune cell activation, and detecting the presence or absence of the detectable label of the cleavage product, wherein the presence of the detectable label indicates an area of granzyme-secreting immune cell activation.

In some embodiments, the present disclosure provides methods of making a molecule useful in delivery of a cargo moiety to a phospholipid bilayer, the methods including synthesizing the molecule as described above, wherein X^{1a} is present, and attaching a cargo moiety to the nucleophilic moiety of X^{1a}, wherein a molecule useful in delivery of a cargo moiety to a phospholipid bilayer is produced. In some embodiments, the synthesizing involves culturing a recombinant host cell comprising an expression construct encoding the molecule. In some embodiments, the synthesizing is by chemical synthesis.

### BRIEF DESCRIPTION OF THE FIGURES

FIG. 1A-1F: The development and in vitro characterization of GRIP B, a restricted interaction peptide to measure GZMB proteolysis in vivo with imaging. (A) A schematic showing a generalized structure of a restricted interaction peptide, and the in vivo mechanism of action. Cleavage of the full length pro-form by a dedicated endoprotease liberates a tagged (e.g., radiolabeled) antimicrobial peptide, which irreversibly interacts with nearby phospholipid membranes. Thus, the stable accumulation of peptide at extended time points post injection (i.e., hours, not seconds) can reflect the relative units of enzyme activity in a region of interest. (B) A schema showing the workflow of the MSP-MS study to identify a GZMB cleavage sequence. Proteolytic products from GZMB activity were produced by incubating the enzyme with a physicochemically diverse library of 228 tetradeca-peptides. Peptide sequencing by LC-MS/MS allowed for the determination of GZMB generated cleavages. (C) An iceLogo showing the consolidated results of an MSP-MS analysis of the P4-P4′ substrate preferences for human GZMB. (D) A plot showing the Michaelis-Menten kinetics of human granzyme B proteolysis of the IEPDVSVQ (SEQ ID NO:64) peptide. Coverage of the non-prime and prime sites of GZMB yielded an optimized substrate with improved catalytic turnover by approximately 2-fold compared to IEPD alone. (E) The final amino acid sequence of GRIP B. Membrane binding domain (SEQ ID NO:3), Granzyme B specific substrate (SEQ ID NO:57), Peptide masking domain (SEQ ID NO:7). (F) Data showing high specificity of the substrate FVQWFSKFLGK (SEQ ID NO:3) for Granzyme B compared to thrombin, caspase 3, caspase 8, granzyme K, MMP9, and C1S.

FIG. 2A-2D: In vitro mechanism of action studies and the synthesis of ⁶⁴Cu-GRIP B. (A) Mean fluorescence intensity data showing the extent of cell labeling by 5FAM-GRIP B in the presence or absence of GZMB. The data were collected using MC28 cells in triplicate. *P<0.01. (B) A bar graph representing the extent of red blood cell lysis due to treatment with vehicle (0.1% DMSO), the full length GRIP B pro-peptide, and the proteolytically activated truncated peptide. Triton-X is included as a positive control. (C) An HPLC trace showing the overlay of the radioactive trace (blue) with the UV trace of the DOTA-GRIP B precursor. The trace was collected 30 min after the start of the reaction. (D) A radioactive HPLC trace showing the conversion of ⁶⁴Cu-GRIP B to one major product after a 30 min incubation with 400 nM recombinant human GZMB.

FIG. 3A-3E: ⁶⁴Cu-GRIP B detects T cell activation in vivo elicited by immune checkpoint inhibition. (A) A time activity curve showing the renal clearance of ⁶⁴Cu-GRIP B in a male C57B16 mouse bearing a subcutaneous CT26 tumor. (B) Representative transaxial CT and PET/CT images showing the accumulation over time of ⁶⁴Cu-GRIP B in a CT26 tumor exposed to anti-PD1 and anti-CTLA4 CPI. Also shown is the uptake of ⁶⁴Cu-GRIP B in a tumor bearing mouse treated with vehicle. (C) A time activity curve from a dynamic PET acquisition showing the tumoral uptake of ⁶⁴Cu-GRIP B in CT26 tumors from mice treated with vehicle or CPI. (D) A plot showing the % change in ⁶⁴Cu-GRIP B uptake per organ in treated versus untreated mice. (E) Digital autoradiography and immunofluorescence showing the co-localization of ⁶⁴Cu-GRIP B with GZMB and T cells within CT26 tumor slices from mice exposed to vehicle or CPI.

FIG. 4A-4F: ⁶⁴Cu-GRIP B biodistribution in vivo is dependent on GZMB proteolytic activity. (A) A bar graph summarizing the post treatment effects on the tumoral uptake of ⁶⁴Cu-GRIP B (or ⁶⁴Cu-L-GRIP B) and ⁶⁴Cu-D-GRIP B, an uncleavable negative control tracer bearing D-amino acids in the GZMB cut site. Three cohorts of mice bearing subcutaneous CT26, MC38 or EMT6 mice were studied. CT26 and MC38 were implanted in male C57B16 mice, and EMT6 were implanted in female Balb/c mice. *P<0.05, **P<0.01. (B) Representative transaxial PET/CT and CT images from the MC38 cohort showing the tumoral uptake of ⁶⁴Cu-L-GRIP B and ⁶⁴Cu-D-GRIP B in mice treated with vehicle or CPI. (C) A bar graph showing the post treatment effects on the splenic uptake of ⁶⁴Cu-GRIP B and ⁶⁴Cu-D-GRIP B in mice treated with vehicle versus CPI. These data were taken from the CT26 cohort and similar trends were observed in the other mouse cohorts. *P<0.01 (D) Autoradiography and H&E showing the relative intensity of ⁶⁴Cu-L-GRIP B and ⁶⁴Cu-D-GRIP B uptake in spleen sections. (E) A bar graph summarizing the post treatment effects on the tumoral and splenic uptake of ⁶⁴Cu-GRIP B in germline GZMB−/− treated with vehicle or CPI. The GZMB−/− were inoculated with CT26 tumors for this study.

FIG. 5A-5B: Post treatment changes in tumoral uptake of ⁶⁴Cu-GRIP B correlates with the magnitude of volumetric tumor response to CPI in wild type mice, but not in GZMB−/− mice. (A) Scatter plots showing the correlation between fold change in tumor volume from day 11 to day 0 and ⁶⁴Cu-GRIP B tumoral uptake (left) or tumor to blood ratio (right). The data were collected from two cohorts of wild type mice bearing CT26 tumors. (B) Scatter plots showing the correlation between fold change in tumor volume from day 11 to day 0 and ⁶⁴Cu-GRIP B tumoral uptake (left) or tumor to blood ratio (right). The data were collected from two cohorts of GZMB−/− mice bearing CT26 tumors.

FIG. 6A-6E: ⁶⁴Cu-GRIP B PET detects secreted GZMB elicited by an endotoxin mediated inflammatory response. (A) Representative ⁶⁴Cu-GRIP B PET/CT studies showing higher radiotracer accumulation in the lungs of mice treated with 0.1 or 3.0 mg/kg LPS compared to mice that received sham. (B) Region of interest analysis of the right lung lobe shows significantly higher radiotracer uptake in LPS treated versus sham treated mice (n=3/arm). *P<0.01 (C) Autoradiography, immunofluorescence, and H&E of the right lung lobe shows higher tracer accumulation in the treated lung, as well as higher GZMB and CD3 staining. (D) A bar graph showing the percent change in radiotracer uptake per organ between the LPS versus sham treated mice (n=4/arm). All changes were determined to be statistically significant, P<0.05. (E) Representative maximum intensity projections showing the systemwide changes in tracer biodistribution due to treatment with 3.0 mg/kg LPS.

FIG. 7A-7B: ⁶⁴Cu-GRIP B PET/CT detects granzymes secreted from activated CAR T cells used in cell based therapy in mice bearing subcutaneous RAJI tumors. Data depicted show images and region of interest (ROI) analysis gathered at 4 hours post injection.

FIG. 8A-8C: ⁶⁴Cu-GRIP B PET/CT detects granzymes secreted from activated CAR T cells used in cell based therapy in mice bearing orthotopic RAJI tumors in liver. Data depicted show images and region of interest analysis gathered at 4 hours post injection. ROI and post mortem dosimetry show CD19 CAR Ts induce ⁶⁴Cu-GRIP B uptake in tumor bearing livers.

FIG. 9A-9C: ⁶⁴Cu-GRIP B PET can detect a productive immune response in a pneumonia model. (A) and (B) Mice received an intranasal instillation of virus or sham, and were imaged with 64Cu-GRIP B at 10 days post infection (the time point at which peak recruitment of T cells to the lungs occurs). Radiotracer uptake in infected lungs is very high and significantly different than healthy lungs at 6 hours post radiotracer injection. (C) Relative radiotracer uptake per organ showed that viral infections induce higher radiotracer uptake in numerous tissues, including the spleen, liver, and blood pool (*P<0.01) in a biodistribution study.

FIG. 10A-10B: ⁶⁴Cu-GRIP B can detect granzyme B secreted from activated immune cells attempting to combat bacterial infections.

FIG. 11A-11B: Imaging studies in germline GZMB knockout mice demonstrate that radiotracer uptake in E. Coli abscesses is due to granzyme B.

FIG. 12A-12B: Live E. coli abscesses induce greater radiotracer uptake and are significantly more immunostimulatory than a bolus of the endotoxin LPS.

FIG. 13A-13B: ⁶⁴Cu-GRIP B accumulation in response to S. aureus infection parallels what has been observed in E coli infections. Radiotracer uptake rose rapidly from 0-6 hours post injection and plateaued from 6-24 hours, and uptake in the live bacterial abscess was significantly higher than in the heat killed abscess.

FIG. 14A-14F: Myositis studies performed with P. aeruginosa and K. pneumoniae displayed similar findings to E. coli and S. aureus infection.

FIG. 15A-15D: M. marinum and L. monoctyogenes do not induce ⁶⁴Cu-GRIP B uptake in live bacterial abscesses compared to heat killed controls.

### DEFINITIONS

The terms “polypeptide,” “oligopeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusion proteins with heterologous and homologous leader sequences, with or without N-terminal methionine residues; immunologically tagged proteins; and the like.

The term “membrane-interacting peptide” refers to a peptide molecule having a plurality of nonpolar hydrophobic amino acid residues, and, when unconstrained by a portion Z as described herein, comprises an alpha-helical structure capable of interaction with phospholipid bilayers such as a cell membrane. Such secondary structure may appear before, during or after insertion of the membrane-interacting peptide into the phospholipid bilayer. The composition of membrane-interacting peptides as described herein is not strictly limited to nonpolar hydrophobic amino acid residues, as such peptides may include different types of amino acid residues, for example, polar uncharged, polar basic, or polar acidic amino acid residues as well.

The term “antimicrobial polypeptide” refers to a type of membrane-interacting peptide that is derived from a naturally-occurring peptide that exhibits antimicrobial activity in its natural form based on its ability to interact with cell membranes. It is understood that the term “antimicrobial polypeptide” as used herein does not require or imply that the polypeptides so described have antimicrobial activity. Any peptide shown to spontaneously interact with and potentially insert into phospholipid membranes are included in this category. For example, spontaneously inserting membrane interaction peptides from naturally occurring transmembrane proteins may be applied. Antimicrobial polypeptides are well known in the art, and include, for example, polypeptides in the temporin family of proteins.

The term “promolecule” as used herein refers to a molecule whose activity is restricted because the individual portions of the molecule are linked together, therefore limiting or restricting the activity that the individual portions may have when not linked to one another. The activity of the individual portions of a promolecule is unleashed upon cleavage or disruption of the bonds that hold the individual portions together. The promolecules of the present disclosure do not inhibit activity of the granzyme.

The term “enzyme-activated” refers to a molecule whose behavior is modified by an enzyme. Many activating enzymes fall under the class of hydrolases EC 3.1 to EC 3.13 or peptidases EC 3.4 to 3.99 in the Nomenclature Committee of the International Union of Biochemistry and Molecular Biology (NC-IUBMB). Example enzyme activities include those that act upon bonds of the type ether, peptide, carbon-nitrogen, acid anhydrides, carbon-carbon, halide, phosphorus-nitrogen, sulfur-nitrogen, carbon-phosphorus, sulfur-sulfur, carbon-sulfur.

The term “non-standard amino acid” means any molecule other than a naturally-occurring amino acid molecule that can be incorporated into a peptide backbone of a polypeptide in lieu of a naturally-occurring amino acid residue in a polypeptide. Non-limiting examples of such non-standard amino acids include: hydroxylysine, desmosine, isodesmosine, or others.

The term “modified amino acid” means any naturally-occurring amino acid that has undergone a chemical or biochemical modification, such as a post-translational modification. Non-limiting examples of modified amino acids include: methylated amino acids, (e.g. methyl histidine, methylated lysine) acetylated amino acids, amidated amino acids, formylated amino acids, hydroxylated amino acids, phosphorylated amino acids, or others.

As used herein, “homologues” or “variants” refers to protein sequences that are similar based on their amino acid sequences. Homologues and variants include proteins that differ from naturally-occurring sequences by one or more conservative amino acid substitutions.

As used herein, the term “conservative amino acid substitution” means a substitution of an amino acid residue for another amino acid residue having similar chemical properties.

The term “treatment” as used herein means that at least an amelioration of the symptoms associated with a disease or condition afflicting the subject is achieved, where amelioration refers to at least a reduction in the magnitude of a parameter, e.g., a symptom, associated with the disease or condition being treated. As such, treatment includes situations where the condition, or at least symptoms associated therewith, are reduced or avoided.

It will be appreciated that throughout the present disclosure reference is made to amino acids according to the single letter or three letter codes. For the reader's convenience, the single and three letter amino acid codes are provided below. In addition, the amino acid residues provided below are divided into categories based on their chemical properties. The headings provided in the table below (Nonpolar, Hydrophobic; Polar, Uncharged; Polar, Acidic; and Polar, Basic) are used to refer generally to amino acid residues having the identified chemical properties.

Nonpolar, Hydrophobic Residues

| | | |
|---|---|---|
| Alanine | Ala | A |
| Valine | Val | V |
| Leucine | Leu | L |
| Isoleucine | Ile | I |
| Phenylalanine | Phe | F |
| Tryptophan | Trp | W |
| Methionine | Met | M |
| Proline | Pro | P |

Polar, Acidic

| | | |
|---|---|---|
| Aspartic Acid | Asp | D |
| Glutamic Acid | Glu | E |

Polar, Uncharged Residues

| | | |
|---|---|---|
| Glycine | Gly | G |
| Serine | Ser | S |
| Threonine | Thr | T |
| Cysteine | Cys | C |
| Tyrosine | Tyr | Y |
| Asparagine | Asn | N |
| Glutamine | Gln | Q |

Polar, Basic

| | | |
|---|---|---|
| Lysine | Lys | K |
| Arginine | Arg | R |
| Histidine | His | H |

The terms “nucleic acid molecule” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

The term “heterologous” refers to two components that are defined by structures that can be derived from different sources. For example, where “heterologous” is used in the context of a polypeptide, the polypeptide includes operably linked amino acid sequences that can be derived from polypeptides having different amino acid sequences (e.g., a first amino acid sequence from a first polypeptide and a second amino acid sequence from a second polypeptide). Similarly, “heterologous” in the context of a polynucleotide encoding a chimeric polypeptide includes operably linked nucleic acid sequences that can be derived from different genes (e.g., a first component from a nucleic acid encoding a first portion of a peptide according to an embodiment disclosed herein and a second component from a nucleic acid encoding a second portion of a peptide disclosed herein).

“Derived from” in the context of an amino acid sequence or polynucleotide sequence (e.g., a polypeptide derived from an antimicrobial peptide) is meant to indicate that the polypeptide or nucleic acid has a sequence that is based on that of a reference polypeptide or nucleic acid, and is not meant to be limiting as to the source or method in which the protein or nucleic acid is made.

The term “operably linked” refers to functional linkage between molecules to provide a desired function. For example, “operably linked” in the context of a polypeptide refers to a functional linkage between amino acid sequences (e.g., of different domains) to provide for a described activity of the polypeptide. “Operably linked” in the context of nucleic acids refers to a functional linkage between nucleic acids to provide a desired function such as transcription, translation, and the like, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide.

As used herein in the context of the structure of a polypeptide, “N-terminus” and “C-terminus” refer to the extreme amino and carboxyl ends of the polypeptide, respectively, while “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Isolated” refers to a protein of interest (e.g., a membrane-interacting peptide) that, if naturally occurring, is in an environment different from that in which it may naturally occur. “Isolated” is meant to include proteins that are within samples that are substantially enriched for the protein of interest and/or in which the protein of interest is partially or substantially purified. Where the protein is not naturally occurring, “isolated” indicates the protein has been separated from an environment in which it was made by either synthetic or recombinant means.

“Enriched” means that a sample is non-naturally manipulated (e.g., by an experimentalist or a clinician) so that a protein of interest is present in a greater concentration than the concentration of the protein in the starting sample, such as a biological sample (e.g., a sample in which the protein naturally occurs or in which it is present after administration), or in which the protein was made (e.g., as in a bacterial protein and the like).

“Substantially pure” indicates that an entity makes up greater than about 50% of the total content of the composition (e.g., total protein of the composition), or greater than about 60% of the total protein content. For example, a “substantially pure” peptide refers to compositions in which at least 75%, at least 85%, at least 90% or more of the total composition is the entity of interest (e.g. 95%, 98%, 99%, greater than 99%), of the total protein. The protein can make up greater than about 90%, or greater than about 95% of the total protein in the composition.

The term “binding” refers to a direct association between two molecules, due to, for example, covalent, electrostatic, hydrophobic, and ionic and/or hydrogen-bond interactions, including interactions such as salt bridges and water bridges.

The term “nucleophilic moiety” as used herein refers to a functional group, which comprises a nucleophilic reactive group. A nucleophilic reactive group comprises at least one pair of free electrons that is able to react with an electrophile. Examples of nucleophilic moieties include sulfur nucleophiles, such as thiols, thiolate anions, anions of thiolcarboxylate, anions of dithiocarbonates, and anions of dithiocarbamates; oxygen nucleophiles, such as hydroxide anion, alcohols, alkoxide anions, and carboxylate anions; nitrogen nucleophiles, such as amines, azides, and nitrates; and carbon nucleophiles, such as alkyl metal halides and enols.

The terms “patient” or “subject” as used interchangeably herein can refer to a human or to a non-human animal, e.g. a mammal, including humans, primates, domestic and farm animals, and zoo, sport, laboratory, or pet animals, such as horses, cows, dogs, cats, rodents, and the like.

### DETAILED DESCRIPTION

Before the invention of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the invention belongs. Although any invention similar or equivalent to those described herein can also be used in the practice or testing of the invention, representative illustrative methods and materials are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present invention is not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present invention and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Overview

The present disclosure generally provides activatable and detectable membrane-interacting peptides that can be used to identify areas of a subject that are associated with a particular biological activity, e.g., proteolysis. Following activation, the promolecules of the present disclosure are capable of forming alpha-helical structures that interact with and insert into phospholipid bilayers, such as cell membranes. The present disclosure also provides methods of use of such compounds.

The compounds of the present disclosure find use in, for example, methods relating to the assessment of granzyme activity. For example, an activatable membrane-interacting peptide having a portion X² that is cleavable by the enzyme granzyme can be administered to a subject. In this example, exposure of the molecule to an area of granzyme activity in the subject results in cleavage at X² to generate a cleavage product containing portion A, which cleavage product is capable of inserting into phospholipid bilayers in the area of granzyme activity. Detection of this cleavage product in phospholipid bilayers can be accomplished by imaging of the tissue(s) suspected of being associated with granzyme activity to image a detectable moiety attached to portion A through portion X^{1a}. The presence of granzyme activity in a subject can also be assessed qualitatively and/or quantitatively by detection of the cleavage product containing portion Z, which may be facilitated by moieties attached as portion X^{1b}.

The compositions of the present disclosure can be used in a variety of methods, including, e.g., use in directly imaging active clotting, infection, or malignancy within a subject.

### Restricted Interaction Peptides

The promolecules of the present disclosure are of the general structure, from N-terminus to C-terminus or from C-terminus to N-terminus:

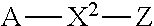

where
A is a membrane-interacting peptide region having a plurality of nonpolar hydrophobic amino acid residues that, following cleavage from the composition, comprises an alpha-helical structure capable of interacting with a phospholipid bilayer;
Z is an inhibitory peptide region that can inhibit the activity of portion A and, in some embodiments, can facilitate targeted interaction of a promolecule with a specific enzyme; and
X² is a granzyme-cleavable linker that can be cleaved to release cleavage products from the compound.

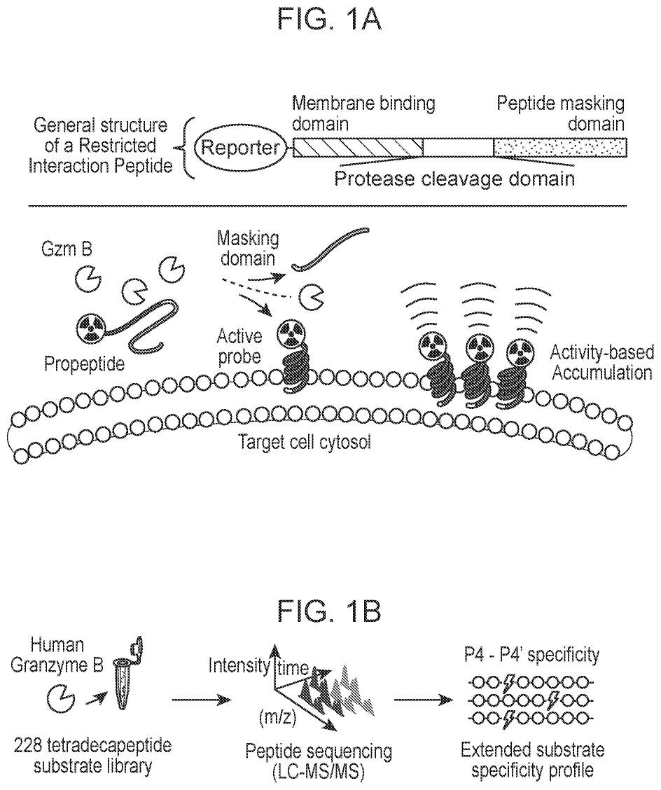

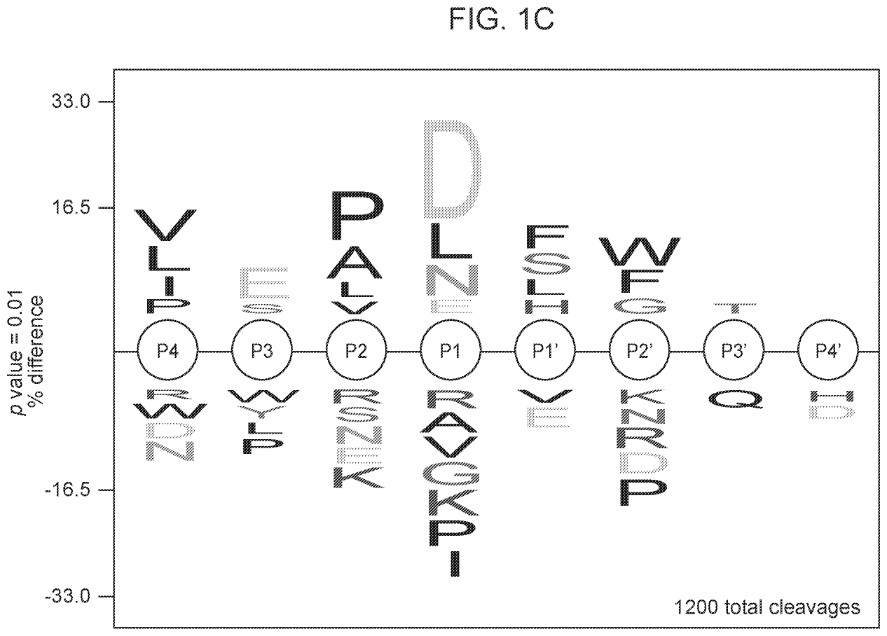

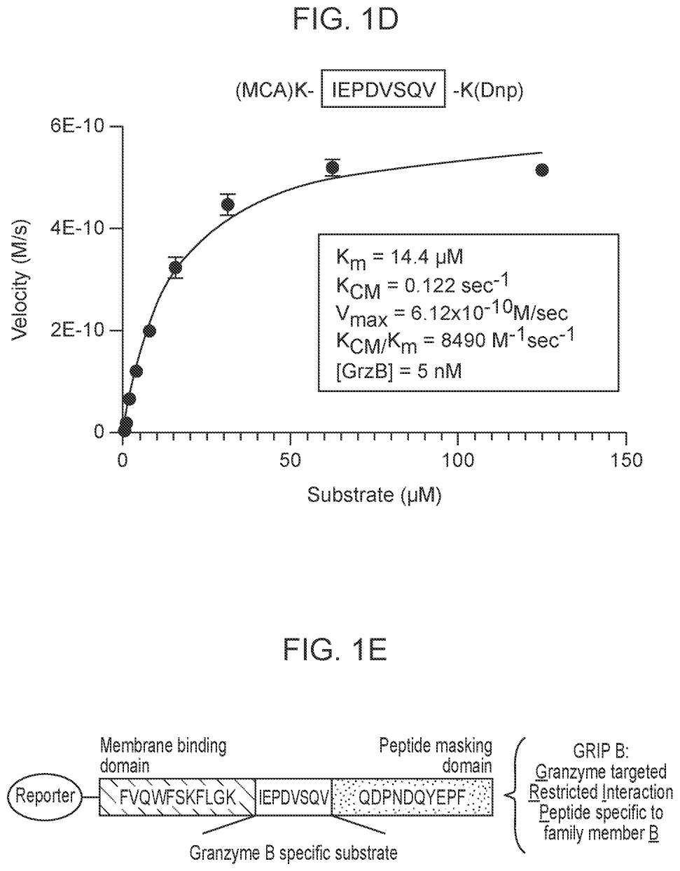

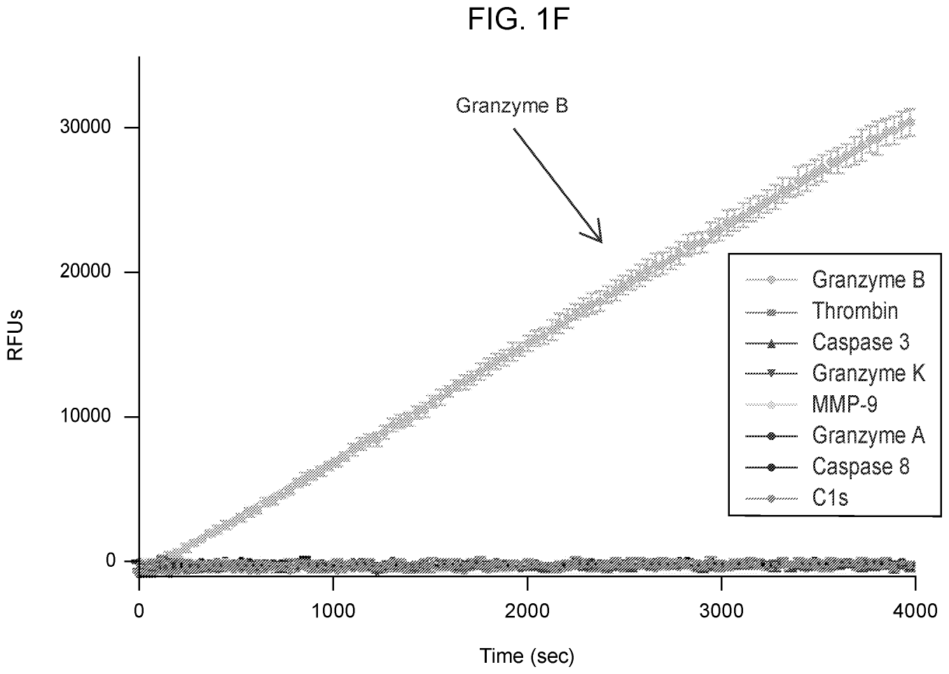

Prior to granzyme-mediated cleavage of the composition at X², the composition acts as a promolecule that does not significantly or detectably associate with phospholipid bilayers. Granzyme-mediated cleavage of X² results in the formation of a cleavage product comprising portion A and a cleavage product comprising portion Z. Following granzyme-mediated cleavage of X², the cleavage product comprising portion A, now unconstrained by portion Z, is free to interact with a phospholipid bilayer (e.g., a cell membrane), and thus accumulate at a site associated with a cleavage-promoting environment (FIG. 1, panel A).

In some embodiments, the promolecules of the present disclosure are of the general structure, from N-terminus to C-terminus or from C-terminus to N-terminus:

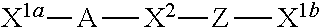

where
A, X², and Z are as described above; and
X^{1a} and X^{1b} are optionally-present chemical handles that facilitate conjugation of various moieties to the compound.

Detection of cleavage products comprising portion A or portion Z can be accomplished by detection of a detectable moiety attached through chemical handle X^{1a} or X^{1b}, or by other methods, e.g., detection using an antibody that specifically binds to an amino acid sequence of the cleavage product.

The various features of the compounds and methods of the present disclosure are described in more detail below.

The overall length of the intact structure X^{1a}-A-X²—Z—X^{1b} may vary based on the sizes of the individual portions that are used to assemble a given molecule. In some embodiments, the overall size of the intact structure is up to about 15 amino acids in length. In some embodiments, the overall length of the intact structure is up to about 20, up to about 30, up to about 40, up to about 50, up to about 60, up to about 70, up to about 80, up to about 90, up to about 100, or up to about 110 amino acids in length. In some embodiments, the overall length of the intact structure may be from about 15 to about 20, about 20 to about 30, about 30 to about 40, about 40 to about 50, about 50 to about 60, about 60 to about 70, about 70 to about 80, about 80 to about 90, about 90 to about 100, or about 100 to about 110 amino acids in length. The overall length of the intact structure is no more than about 115 amino acids in length.

The intact structure X^{1a}-A-X²—Z—X^{1b} may be referred to herein as a “promolecule.” Portion A of the promolecule does not significantly interact with phospholipid bilayers due to the presence of portion Z in the promolecule. Without being held to theory, portion Z inhibits the phospholipid bilayer interacting properties of portion A by preventing portion A from forming an alpha-helical structure when portion A and portion Z are linked together by portion X². Following cleavage of X², portion Z is separated from portion A, allowing the cleavage product comprising portion A to undergo a conformational change such that at least portion A can form a regular structure such as that of an alpha-helical structure. In the alpha-helical conformation, portion A spontaneously interacts with phospholipid bilayers, e.g., by inserting into the phospholipid bilayer.

One of ordinary skill in the art will appreciate that the promolecules of the present disclosure can be adapted for use in a variety of settings, e.g., by providing for granzyme-cleavable linkers that differ in conditions that provide for cleavage. In some embodiments, a promolecule has the structure, from N-terminus to C-terminus or from C-terminus to N-terminus, A-X²—Z. In some embodiments, a promolecule has the structure, from N-terminus to C-terminus or from C-terminus to N-terminus, X^{1a}-A-X²—Z. In some embodiments, a promolecule has the structure, from N-terminus to C-terminus or from C-terminus to N-terminus, A-X²—Z—X^{1b}. In some embodiments, a promolecule has the structure, from N-terminus to C-terminus or from C-terminus to N-terminus, X^{1a}-A-X²—Z—X^{1b}. As disclosed herein, the various embodiments of portions X^{1a}, A, X², Z, and X^{1b} may be freely interchanged to form a molecule having any of the above-described features. In some embodiments, multiple copies of X^{1a} or X^{1b} may be incorporated to enhance detection sensitivity or pharmacological properties.

### Properties of Restricted Interaction Peptides

As described above, the promolecules of the present disclosure have the general structure X^{1a}-A-X²—Z—X^{1b}. In order to prevent the membrane-interacting portion (portion A) from interacting with cell membranes prior to activation, the promolecules are designed to have an isoelectric point (pI) of 7 or lower. The isoelectric point is the pH at which the net charge on a peptide molecule is zero. The pI of the full-length promolecules of the present disclosure can be modulated by adjusting the pI of one or more of the individual portions X^{1a}, A, X², Z, or X^{1b} that make up the promolecule, or by chemically modifying any or all portions of the compound (e.g., by phosphorylation or sulfation to impart additional negative charge).

The pI of a peptide can be modulated by substituting, eliminating, or introducing amino acid residues in order to change the overall net charge of the peptide. Decreasing the net charge of a peptide reduces its pI value. For example, eliminating one or more positively charged amino acid residues (e.g. K, R, or H) or replacing such residues with uncharged or negatively charged residues reduces the pI value of the peptide.

The pI of a given peptide can be readily determined by using a computer algorithm for pI estimation (e.g., Protein Calculator, Scripps Institute). Such computer algorithms are readily available to the public via the internet and can determine a theoretical pI value for a peptide based on its amino acid sequence. Promolecules of the present disclosure are designed to have a theoretical pI value less than or equal to 7.

The promolecules of the present disclosure are generally designed to have an overall net charge preferably less than or equal to about zero. This can be accomplished, for example, by substituting, eliminating, or introducing various amino acid residues in the polypeptide sequences of portion A or portion Z, or by introducing charged moieties to portion Z, to neutralize the overall charge of the promolecule. Charged amino acid residues or moieties that are introduced to neutralize the charge of other amino acid residues or chemical moieties are placed as close as possible to one another in order to maximize charge-cancelling effects, e.g., a distance of no more than about 40 Angstroms. In some embodiments, promolecules of the present disclosure need not have an overall charge of less than or equal to about zero if, for example, the propensity of the membrane interaction segment to spontaneously insert into phospholipid membranes is limited.

Cargo moieties that are optionally conjugated to the promolecules of the present disclosure may be charged, and therefore may impact the pI value and the overall net charge of a promolecule, thus impacting the restriction in membrane-interacting activity. The charge on a particular cargo moiety added to a promolecule will generally cancel or neutralize the overall net charge of the promolecule that it is conjugated to and reduce the overall pI to a value of seven or lower. For example, a promolecule having an overall net charge of +1 could be conjugated to a detectable moiety having a charge of −1 to produce a molecule having an overall net charge of zero. Water soluble fluorescent dyes, such as those in the cyanine dye family, including Cy3, Cy5, and Cy7, are particularly useful in this regard due to their zwitterionic nature from two negatively-charged sulphate groups and a tertiary amine group. Metal chelating moieties, such as 1,4,7,10-tetraazacyclododecane-1,4,7,10-tetraacetic acid (DOTA) or diethylene triamine pentaacetic acid (DTPA) capable of binding radioisotopes Gallium-68 or Technetium-99m are zwitterionic as well, bearing multiple positively- and negatively-charged moieties. Metal binding to DOTA or DTPA occurs through the amine groups, thus allowing these entities to impart a charge of up to −4.

Portion Z's ability to inhibit or prevent portion A from interacting with phospholipid bilayers can also be modulated by changing the overall length of portion Z. This can be done, for example, by adding amino acid residues to portion Z, or by conjugating a molecule, such as a water-soluble polymer, to portion Z. In some embodiments, negatively charged amino acids or similar chemical modifications, such as phosphates or sulphate moieties, are added to portion Z. In some embodiments, polyethylene glycol is conjugated to portion Z in order to increase the length of portion Z and enhance its ability to inhibit portion A from interacting with cell membranes prior to activation. In some embodiments, whole proteins (e.g. albumin) may be conjugated to portion Z. In certain embodiments, a polymer or protein that is conjugated to portion Z may also increase the circulating half-life of the promolecule. In some embodiments, the promolecules of the present disclosure may be conjugated to polymers having branched, dendrimeric, or otherwise polyvalent architecture.

### Portion A—Membrane-Interacting Peptides

The membrane-interacting peptides of the present disclosure comprise amino acid sequences that are capable of forming alpha-helical structures, e.g., upon contacting an environment with a lower dielectric constant than water. Following granzyme-mediated cleavage of X², the cleavage product comprising portion A comprises an alpha-helical structure that is capable of inserting into a phospholipid bilayer in the vicinity of the cleavage-promoting environment. Without being held to theory, the alpha-helical structure of portion A may be present in the molecule prior to cleavage but, due to constraint by portion Z, is unable to insert into a phospholipid bilayer. The presence of portion A and/or portion Z constrains portion A such that portion A does not form an alpha-helical structure sufficient to allow for significant or detectable insertion into a phospholipid bilayer.

An alpha helix is a common motif in the secondary structure of proteins, and generally comprises a right-handed coiled or spiral conformation that is stabilized by hydrogen bonds in which the N—H group of a first amino acid residue forms a hydrogen bond with the C═O group of an amino acid residue located four residues away in the polypeptide chain. A typical alpha helix comprises approximately 3.6 amino acid residues per turn of the helix, and is a tightly-packed structure. The side chains of the amino acid residues that make up an alpha helix face the outside of the helix. Different amino acid sequences have different propensities for forming alpha helices due, in part, to the differing chemical properties of the amino acid side chains.

As described above, promolecules of the present disclosure generally comprise a membrane-interacting peptide portion A. Portion A may be derived from a naturally-occurring polypeptide, or may be a variant of a naturally-occurring polypeptide. The overall length of portion A can be, for example, about 5 up to about 10 amino acids, or can be up to about 15, up to about 20, up to about 25, or up to about 30 amino acids. Portion A may range in size from about 5 to about 10 amino acids in length, or may be about 10 to about 15, about 15 to about 20, about 20 to about 25, or about 25 to about 30 amino acids in length. Portion A is no longer than about 35 amino acid residues in length.

Membrane-interacting peptides generally comprise a plurality of nonpolar, hydrophobic amino acid residues (e.g., alanines, valines, leucines, isoleucines, phenylalanines, tryptophans, methionines, or prolines), but may comprise other types of amino acids as well, such as polar uncharged, polar acidic, and/or polar basic amino acid residues. In general, the membrane-interacting peptides of the present disclosure comprise fewer than 5 polar basic amino acid residues. In some embodiments, the amino acid sequence of portion A comprises multiple regions of two to three contiguous nonpolar hydrophobic amino acid residues interspersed with regions of one to two contiguous polar uncharged, polar acidic, or polar basic residues. After cleavage of X², portion A undergoes a conformational change, typically forming an alpha-helical structure that readily interacts with cell membranes (e.g., membranes present in the cells of eukaryotic, prokaryotic or archael organisms, or artificial membranes of detergent micelles or liposomes of varying compositions, including synthetic polymers).

### Antimicrobial Peptides

Antimicrobial peptides that elicit their effects through membrane interaction are well known in the art, and include examples such as the temporin family of proteins, which can be naturally obtained from the skin of frogs belonging to the Rana temporaria species. Antimicrobial peptides generally comprise fewer than about 30 amino acid residues and, under physiological conditions, contain alpha-helical structures having nonpolar hydrophobic amino acid residues that facilitate their interaction with the phospholipid bilayers of cell membranes. Such interactions may generally include types ranging from structured barrel-stave pores to broadly-defined detergent-like behavior. In some embodiments of the present disclosure, an amino acid sequence of a naturally-occurring antimicrobial peptide is utilized as a membrane-interacting peptide. In other embodiments, a membrane-interacting peptide that comprises modifications relative to a naturally-occurring antimicrobial peptide, e.g., elimination, introduction, or substitution of one or more amino acid residues, addition of chemical modifications such as disulfide bonds, or other chemical modifications (e.g., amidation), is utilized as a membrane-interacting peptide. In other embodiments, the peptide sequence is capable of spontaneous membrane interaction and/or insertion, but is not associated with membrane disrupting activity.

Examples of antimicrobial peptides are provided below.

### Modifications to Membrane-Interacting Peptides

Antimicrobial peptides or portions thereof may be incorporated into the compounds of the present disclosure in their naturally-occurring form, or may be modified to alter their chemical properties and adapt such for a desired use. For example, the membrane-interaction potential of antimicrobial peptides may be strengthened or weakened by, e.g., adding, eliminating or substituting certain amino acid residues in the protein sequence. Such additions, eliminations, or substitutions can be made, e.g., to introduce charged amino acid residues, to eliminate charged amino acid residues, to introduce hydrophobic amino acid residues, to eliminate hydrophobic amino acid residues, etc.

In some embodiments, an antimicrobial peptide sequence may be altered by chemically modifying the peptide with disulfide bonds or other chemical modifications (e.g. amidation). Many antimicrobial peptides are naturally produced with such modifications to improve the potency of their interactions with phospholipid membranes and resistance to proteolysis.

### Temporins

In some embodiments, portion A comprises a protein from the Temporin family. Proteins in the Temporin family generally range from about 10 up to about 14 amino acids in length. The consensus sequence for the Temporin family of proteins showing the most abundant amino acid found at each position is: FLP(I/L)IASLL(S/G)KLL (SEQ ID NO:8). The consensus sequence for the Temporin family of proteins showing the general amino acid type found at each position is: X^{a}X^{b}X^{c}X^{d}X^{e}X^{f}Y^{a}X^{g}X^{h}Y^{b}Y*XⁱX^{j}, where X^{a}, X^{b}, X^{c}, X^{d}, X^{e}, X^{f}, X^{g}, X^{h}, Xⁱ, and X^{j} are hydrophobic amino acid residues, Y^{a} and Y^{b} are hydrophilic amino acid residues, and Y* is a charged amino acid residue. The table below shows the amino acid sequences of several Temporin and Temporin-like peptides that are useful in the promolecules and methods of the present disclosure.

As described above, antimicrobial peptide sequences may be altered by eliminating or substituting one or more of the amino acid residues. For example, in some embodiments, a membrane-interacting peptide comprises Temporin-L, whose amino acid sequence is FVQWFSKFLGRIL (SEQ ID NO:1). In other embodiments, a membrane-interacting peptide comprises a derivative of Temporin-L having the amino acid sequence FVQWFSKFLGKLL (SEQ ID NO:2), wherein amino acid residues R and I at positions 11 and 12 of the Temporin-L sequence have been replaced with amino acid residues K and L, respectively. In some embodiments, a membrane-interacting peptide comprises a derivative of Temporin-L having the amino acid sequence FVQWFSKFLGK (SEQ ID NO:3), wherein amino acid residue R at position 11 of the Temporin-L sequence has been replaced with amino acid residue K, and amino acid residues I and L at positions 12 and 13 of the Temporin-L sequence are not present.

TABLE 1
Amino acid sequences of Temporin and Temporin-like peptides thirteen
amino acids in length. Longer and shorter members of the family
have also been described but are not included in this table.

| Peptide Name | Amino Acid Sequence | | | | | | | | | | | | | | | ID NO |
|---|---|---|---|---|---|---|---|---|---|---|---|---|---|---|---|---|
| Temporin-A | — | F | L | P | L | I | G | R | V | L | S | G | I | L | — | 9 |
| Temporin-B | — | L | L | P | I | V | G | N | L | L | K | S | L | L | — | 10 |
| Temporin-C | — | L | L | P | I | L | G | N | L | L | N | G | L | L | — | 11 |
| Temporin-D | — | L | L | P | I | V | G | N | L | L | N | S | L | L | — | 12 |
| Temporin-E | — | V | L | P | I | I | G | N | L | L | N | S | L | L | — | 13 |
| Temporin-F | — | F | L | P | L | I | G | K | V | L | S | G | I | L | — | 14 |
| Temporin-G | — | F | F | P | V | I | G | R | I | L | N | G | I | L | — | 15 |
| Temporin-H | — | L | S | P | — | — | — | N | L | L | K | S | L | L | — | 16 |
| Temporin-K | — | L | L | P | — | — | — | N | L | L | K | S | L | L | — | 17 |
| Temporin-L | — | F | V | Q | W | F | S | K | F | L | G | R | I | L | — | 1 |
| Temporin-1Ca | — | F | L | P | F | L | A | K | I | L | T | G | V | L | — | 18 |
| Temporin-1Cb | — | F | L | P | L | F | A | S | L | I | G | K | L | L | — | 19 |
| Temporin-1Cc | — | F | L | P | F | L | A | S | L | L | T | K | V | L | — | 20 |
| Temporin-1Cd | — | F | L | P | F | L | A | S | L | L | S | K | V | L | — | 21 |
| Temporin-1Ce | — | F | L | P | F | L | A | T | L | L | S | K | V | L | — | 22 |
| Temporin-1Ga | S | I | L | P | T | I | V | S | F | L | S | K | V | F | — | 23 |
| Temporin-1Gb | S | I | L | P | T | I | V | S | F | L | S | K | F | L | — | 24 |
| Temporin-1Gc | S | I | L | P | T | I | V | S | F | L | T | K | F | L | — | 25 |
| Temporin-1Gd | F | I | L | P | L | I | A | S | F | L | S | K | F | L | — | 26 |
| Temporin-1La | — | V | L | P | L | I | S | M | A | L | G | K | L | L | — | 27 |
| Temporin-1Lb | N | F | L | G | T | L | I | N | L | A | K | K | I | M | — | 28 |
| Temporin-1Lc | — | F | L | P | I | L | I | N | L | I | H | K | G | L | L | 29 |
| Temporin-1P | — | F | L | P | I | V | G | K | L | L | S | G | L | L | — | 30 |
| Ranatuerin-5 | — | F | L | P | I | — | A | S | L | L | G | K | Y | L | — | 31 |
| Ranatuerin-6 | — | F | I | S | A | I | A | S | M | L | G | K | F | L | — | 32 |
| Ranatuerin-7 | — | F | L | S | A | I | A | S | M | L | G | K | F | L | — | 33 |
| Ranatuerin-8 | — | F | I | S | A | I | A | S | F | L | G | K | F | L | — | 34 |
| Ranatuerin-9 | F | L | F | P | L | I | T | S | F | L | S | K | V | L | — | 35 |
| Peptide A1 | — | F | L | P | A | I | A | G | I | L | S | Q | L | F | — | 36 |
| Peptide B9 | — | F | L | P | L | I | A | G | L | L | G | K | L | F | — | 37 |

In some embodiments of the present disclosure, a membrane-interacting peptide comprises a Temporin or a Temporin-like peptide listed in Table 1, or a conservative amino acid substitution thereof. In some embodiments of the present disclosure, a membrane-interacting peptide comprises the sequence of Temporin-L (FVQWFSKFLGRIL; SEQ ID NO:1), or a conservative amino acid substitution thereof.

### Protonectin

In some embodiments of the present disclosure, a membrane-interacting peptide comprises Protonectin, having the amino acid sequence ILGTILGLLKGL (SEQ ID NO:5), or a conservative amino acid substitution thereof.

### Japonicins

In some embodiments, a membrane-interacting peptide may comprise a Japonicin or a Japonicin-like peptide listed in Table 2, or a conservative amino acid substitution thereof. In some embodiments of the present disclosure, a membrane-interacting peptide comprises the sequence of Japonicin-1 (FFPIGVFCKIFKTC; SEQ ID NO:38), or a conservative amino acid substitution thereof. Japonicins are naturally obtainable from the skin of the Japanese brown frog Rana japonica and range in length from about 14 up to about 21 amino acid residues. The table below shows the amino acid sequences of several Japonicin and Japonicin-like peptides that are useful in the promolecules and methods of the present disclosure.

TABLE 2
Amino acid sequences of Japonicin and Japonicin-like peptides.

| Peptide Name | Amino Acid Sequence | | | | | | | | | | | | | | | | | | | | | ID NO |
|---|---|---|---|---|---|---|---|---|---|---|---|---|---|---|---|---|---|---|---|---|---|---|
| Japonicin-1 | F | — | — | — | — | — | — | F | P | I | G | V | F | C | K | I | F | K | — | T | C | 38 |
| Japonicin- | F | — | — | — | — | — | — | F | P | L | A | L | L | C | K | V | F | K | — | K | C | 39 |
| 1CDYa | | | | | | | | | | | | | | | | | | | | | | |
| Japonicin- | F | — | — | — | — | — | — | L | L | F | P | L | M | C | K | I | Q | G | — | K | C | 40 |
| 1Npa | | | | | | | | | | | | | | | | | | | | | | |
| Japonicin- | F | — | — | — | — | — | — | V | L | P | L | V | M | C | K | I | L | R | — | K | C | 41 |
| 1Npb | | | | | | | | | | | | | | | | | | | | | | |
| Japonicin-2 | F | G | L | P | M | L | S | I | L | P | K | A | L | C | I | L | L | K | R | K | C | 42 |

In some embodiments of the present disclosure, a membrane-interacting peptide comprises a Japonicin or a Japonicin-like peptide listed in Table 2, or a conservative amino acid substitution thereof.

### Additional Peptides

In addition to the peptides described above, promolecules of the present disclosure may comprise a membrane-interacting peptide listed in the following table, or a conservative amino acid substitution thereof. In some embodiments, the peptides listed in the table below comprise N- and/or C-terminal modifications that may modulate their activity.

TABLE 3
Peptides suitable for use in portion A.

| Peptide Name | Amino Acid Sequence | ID NO |
|---|---|---|
| Combi-1 | RRWWRF | 43 |
| Combi-2 | FRWWHR | 44 |
| Jelleine-1 | PFKLSLHL | 45 |
| Jelleine-2 | TPFKLSLHL | 46 |
| Temporin-SHf | FFFLSRIF | 47 |
| Modified | FFWLSKIF | 48 |
| Temporin-SHF | | |
| Jcpep7 | KVFLGLK | 49 |
| Myxinidin | GIHDILKYGKPS | 50 |
| 1T51 | ILGKIWEGIKSLF | 51 |
| Mastoparan B | LKLKSIVSWAKKVL | 52 |
| K4 | KKKKPLFGLFFGLF | 53 |
| Agelaia-MP | INWLKLGKAIIDAL | 54 |

### Portion Z—Membrane-Interaction Inhibitory Peptide

Compounds of the present disclosure generally comprise portion Z, which inhibits or prevents portion A from interacting with phospholipid bilayers when linked to portion A through portion X². In some embodiments, portion Z also facilitates the interaction of the promolecule with a target enzyme.

Portion Z is generally a polypeptide comprising about 2 up to about 15 amino acid residues in length. In some embodiments, portion Z is up to about 5, up to about 10, up to about 15, up to about 20, up to about 25, up to about 30, up to about 35, up to about 40, up to about 45, or up to about 50 amino acids in length. In some embodiments, portion Z ranges from about 2 to about 5, about 5 to about 10, about 10 to about 15, about 15 to about 20, about 20 to about 25, about 25 to about 30, about 30 to about 35, about 35 to about 40, about 40 to about 45, or about 45 to about 50 amino acids in length. Portion Z is no more than about 55 amino acids in length.

Portion Z may comprise any type of amino acid residue. In some embodiments, portion Z may optionally comprise a detectable moiety to facilitate detection of portion Z following cleavage of X².

### Recognition Domains

Portion Z may be designed to aid in modulating the cleavage of X² and subsequent activation of portion A. In some embodiments, portion Z comprises an amino acid sequence that can be bound by a granzyme that cleaves X². In certain embodiments, recognition of a specific amino acid sequence in portion Z may be required before a granzyme is able to cleave X² and activate the promolecule. Some of the amino acid residues in portion Z may be adjusted to modulate the activity of the promolecule without changing the specificity of interaction between portion Z and the target granzyme.

In some embodiments, portion Z comprises an amino acid sequence derived from naturally occurring physiologic substrates of the enzymatic granzyme activator. In some embodiments, portion Z comprises an amino acid sequence derived from the analysis of screening of combinatorial peptide libraries that may or may not share similarity to physiologic substrates of the enzymatic granzyme activator.

In some embodiments, portion Z comprises the sequence of protease-activated receptor-1 (PAR-1), having the amino acid sequence SFLLRNPNDKYEPFW (SEQ ID NO:55), or a conservative amino acid substitution thereof. In other embodiments, portion Z comprises the amino acid sequence SFLLQDPNDQYEPFW (SEQ ID NO:56), or a conservative amino acid substitution thereof. In some embodiments, portion Z comprises the amino acid sequence QDPNDQYEPF (SEQ ID NO:7), or a conservative amino acid substitution thereof.

### X²— Cleavable Linkers

In the promolecules of the present disclosure, portion A is linked to portion Z through cleavable linker X². In some embodiments, X² comprises a linker that links portion A to portion Z with a single chemical bond. In other embodiments, X² comprises a chimeric linker that links portion A to portion Z through two or more different chemical bonds.

Cleavage of X² produces two cleavage products: a first cleavage product containing portion A and a second cleavage product containing portion Z. In general, X² is cleavable under a pre-selected physiological condition. X² can be selected so that the promolecule is selectively cleaved when exposed to an environment associated with a condition to be diagnosed or detected.

X² may comprise a chemical bond that is subject to cleavage by proteases or other enzymes found on the surface of cells or released near cells having a condition to be diagnosed or detected, such immune system activation or dysregulation, or by other conditions or factors.

X² may comprise an amino acid or a peptide. When X² comprises a peptide, the peptide may be of any suitable length, such as, for example, about 2 up to about 5, up to about 10, up to about 15, up to about 20, up to about 25, or up to about 30 amino acid residues in length. In some embodiments, X² is about 2 to about 5, about 5 to about 10, about 10 to about 15, about 15 to about 20, about 20 to about 25, or about 25 to about 30 amino acids in length. X² is no longer than about 35 amino acids in length. A cleavable peptide may include an amino acid sequence recognized and cleaved by a protease, so that proteolytic action of the protease cleaves X².

### Enzymatically-Cleavable Linkers

The design of X² for cleavage by specific conditions, such as by the presence of granzyme activity, allows targeting of promolecule activation to a specific location where such conditions are found. Thus, one way that compounds of the present disclosure provide specific targeting to regions of granzyme activity or regions of activation of granzyme-secreting immune cells is by the design of the linker portion X² to be cleaved by a granzyme. After granzyme-mediated cleavage of X², cleavage products A and Z are formed, and portion A is free to interact with phospholipid bilayers, such as cell membranes, in the vicinity of activation.

X² is a granzyme-cleavable peptide. X² may be cleavable by any granzyme of interest. Non-limiting examples of granzymes for which the peptide may be cleavable include granzyme A, granzyme B, granzyme H, granzyme K, and granzyme M. In certain embodiments, the granzyme is granzyme B, e.g., human granzyme B (UniProtKB—J3KPK2), mouse granzyme B (UniProtKB—P04187), or the like. In some embodiments, X² is a human granzyme B-cleavable peptide having the amino acid sequence IEPDVSQV (SEQ ID NO:57). This amino acid sequence is specifically cleaved by the human enzyme granzyme B. In certain embodiments, the granzyme is granzyme K, e.g., human granzyme K (UniProtKB—P49863), mouse granzyme K (UniProtKB—O35205), or the like. In some embodiments, X² is granzyme K-cleavable peptide having the amino acid sequence WAFRSRYH (SEQ ID NO:58). This amino acid sequence is specifically cleaved by the human enzyme granzyme K. In certain embodiments, the granzyme is granzyme A, e.g., human granzyme A (UniProtKB—P12544), mouse granzyme A (UniProtKB—P11032), or the like. In certain embodiments, the granzyme is granzyme H, e.g., human granzyme H (UniProtKB—P20718), pig granzyme H (UniProtKB—B8XTR8), or the like. In certain embodiments, the granzyme is granzyme M, e.g., human granzyme M (UniProtKB—P51124), mouse granzyme M (UniProtKB—008643), or the like.

### Linkers Cleavable by Granzymes

In some embodiments, X² linkers are susceptible to cleavage by the enzyme granzyme, which is an enzyme involved with the immune response. Granzymes are expressed primarily within the secretory vesicles (i.e., granules) of lymphocytes involved in host defense (e.g., natural killer cells and cytotoxic T lymphocytes). Following lymphocyte docking with a target cell, the lymphocyte degranulates and releases granzymes into the pericellular space. Therefore, granzymes may be used to cleave X² linkers and target detection of granzyme activity or activation of immune cells such as natural killer (NK) cells and cytotoxic T lymphocytes (CTL), using the promolecules of the present disclosure. In areas where a granzyme of interest is present, X² is cleaved, releasing portion A from portion Z and allowing portion A to interact with cell membranes in the vicinity.

In some embodiments, the X² linkers are cleavable by granzyme B. When the X² linker is cleavable by granzyme B, in some embodiments, the linker comprises the amino acid sequence X^{m}XⁿPDX^{o}SX^{p}X^{q}, wherein X^{m} is V, L, or I; Xⁿ is E; X^{o} is F, S, or V; X^{p} is T or Q; and X^{q} is V. According to some embodiments, when the X² linker is cleavable by granzyme B, the linker comprises the amino acid sequence IEPDVSQV (SEQ ID NO:57), LTYDFWIQ (SEQ ID NO:65), PQVDLYDK (SEQ ID NO:66), VVQDKHEI (SEQ ID NO:67), VYADSSEW (SEQ ID NO:68), TMADSQES (SEQ ID NO:69), GHIDHMXX (SEQ ID NO:70), LEQDVWIA (SEQ ID NO:71), LDPDNFKR (SEQ ID NO:72), XXPDFYLG (SEQ ID NO:73), MGPDAFNL (SEQ ID NO:74), LKDDMGXX (SEQ ID NO:75), IWFDYTLK (SEQ ID NO:76), XIGDNVEW (SEQ ID NO:77), XXXDQVNL (SEQ ID NO:78), PQADQWXX (SEQ ID NO:79), PSVDMXXX (SEQ ID NO:80), XNVDWTAP (SEQ ID NO:81), YGYDLQTA (SEQ ID NO:82), HGFDEAHN (SEQ ID NO:83), HSHDSWKA (SEQ ID NO:84), KQDDLMSE (SEQ ID NO:85), SFGDIMEM (SEQ ID NO:86), VNDDVKXX (SEQ ID NO:87), XXXDKQFT (SEQ ID NO:88), or NDVDGGXX (SEQ ID NO:89), where X is any amino acid. In one non-limiting example, the granzyme B cleavable linker comprises the amino acid sequence IEPDVSQV (SEQ ID NO:57). Granzyme B cleavable sequences which may be included in the cleavable linkers of the promolecules of the present disclosure include those provided in the MEROPS database. See www.ebi.ac.uk/merops/index.shtml. See also Rawlings et al. (2018) Nucleic Acids Res 46, D624-D632.

In some embodiments, the X² linkers are cleavable by granzyme K. When the X² linker is cleavable by granzyme K, in some embodiments, the linker has the amino acid sequence X^{r}X^{s}FRSX^{t}X^{u}X^{v}, wherein X^{r} is E or W; X^{s} is F, Y, or A; X^{t} is F, R, or I; X^{u} is Y, P, or T; and X^{v} is W or H. In one non-limiting example, the granzyme K cleavable linker comprises the amino acid sequence WAFRSRYH (SEQ ID NO:58). Granzyme K cleavable sequences which may be included in the cleavable linkers of the promolecules of the present disclosure include those provided in the MEROPS database. See www.ebi.ac.uk/merops/index.shtml. See also Rawlings et al. (2018) Nucleic Acids Res 46, D624-D632.

In some embodiments, X² linkers may be cleaved by other granzymes. In one non-limiting example, when the X² linker is cleavable by granzyme A, the linker comprises the amino acid sequence ASPRAGGK (SEQ ID NO:59). In another non-limiting example, when the X² linker is cleavable by granzyme M, the linker comprises the amino acid sequence KEPLSAEA (SEQ ID NO:60). Additional granzyme cleavable sequences which may be included in the cleavable linkers of the promolecules of the present disclosure include those provided in the MEROPS database. See www.ebi.ac.uk/merops/index.shtml. See also Rawlings et al. (2018) Nucleic Acids Res 46, D624-D632.

### Combinations of Multiple Linkers

In some embodiments, X² comprises an amino acid sequence that provides two or more sites susceptible to cleavage (e.g., by an enzyme), wherein at least one of the sites is susceptible to cleavage by a granzyme. Where a molecule having features of the present disclosure includes an X² linker comprising multiple cleavage sites, separation of portion A from portion Z may require cleavage of multiple bonds within the X² linker, which may take place either simultaneously or sequentially. Such X² linkers may include bonds having different chemical properties or cleavage specificities, so that separation of portion A from portion Z requires that more than one condition or environment (“extracellular signals”) be encountered by the molecule before activation takes place. The cleavage sites may be the same or different, and where different may be referred to herein as a “chimeric” linker. Cleavage of chimeric X² linkers thus serves as a detector of combinations of such extracellular signals.

Chimeric X² linkers may be used to further modulate the targeting of portion A to desired cells, tissue, or regions. Boolean combinations of extracellular signals can be used to broaden or narrow the conditions under which cleavage of X² occurs. Where chimeric X² linkers are used to link portion A to portion Z, the different chemical bonds within the chimeric linker can be arranged in parallel or in series. When arranged in parallel, the cleavage conditions are narrowed, since each bond must be cleaved before portion A may separate from portion Z. When the chemical bonds within the chimeric linker are arranged in series, the cleavage conditions are broadened, since cleavage of any one of the chemical bonds will result in separation of portion A from portion Z.

In some embodiments, a chimeric X² linker comprises a site susceptible to cleavage by a granzyme and a site susceptible to cleavage by a second protease or enzyme. In some embodiments, the second enzyme is the same or a different granzyme.

In some embodiments, a chimeric X² linker comprises a site susceptible to cleavage by a granzyme and a site susceptible to cleavage under reducing conditions. Reducing conditions can be found in regions having reduced oxygen concentration (i.e., hypoxia), such as regions surrounding cancer cells and cancerous tissues, infarct regions, and other hypoxic regions. Examples of sites susceptible to cleavage under hypoxic conditions include those containing a disulfide bond. In a hypoxic environment, free thiols and other reducing agents become available extracellularly, while the oxygen that normally maintains the extracellular environment in an oxidizing state is depleted. This shift in the redox balance promotes reduction and cleavage of a disulfide bond within an X² linker. In addition to disulfide linkages that take advantage of thiol-disulfide equilibria, linkages including quinones that are cleaved when reduced to hydroquinones may be used in an X² linker designed for cleavage in reducing environments.

In some embodiments, a chimeric X² linker comprises a site susceptible to cleavage by a granzyme and a site susceptible to cleavage under in acidic environments. Acidic environments can be found at sites near damaged or hypoxic tissue. Sites susceptible to cleavage in acidic environments can be utilized to target activation of the promolecules of the present disclosure to acidic regions. Such targeting could be achieved with an acid-labile linker (e.g., by including in X² an acetal or vinyl ether linkage, or another linkage that is cleaved under acidic conditions).

For example, in order to detect the presence of a granzyme or hypoxia (i.e., to cleave X² in the presence of either granzyme activity or hypoxia), a chimeric X² linker is designed with the granzyme-sensitive and reduction-sensitive chemical bonds in series, so that cleavage of either bond would suffice to allow separation of portion A from portion Z.

Alternatively, in order to detect the presence of both granzyme activity and hypoxia (i.e., to cleave X² in the presence of granzyme activity and hypoxia but not in the presence of only one of these conditions alone), a dual X² linker could be designed, e.g., a chimeric linker, to place the granzyme sensitive bond between at least one pair of cysteines that are disulfide-bonded to each other (i.e., the chemical bonds in the chimeric X² linker are arranged in parallel). In this case, both granzyme cleavage and disulfide reduction are required in order to allow separation of portions A and Z.

In certain embodiments, promolecules of the present disclosure may have the following formula (I), wherein portion X² comprises a dual linker, which may be a chimeric linker, and has a cyclic structure.

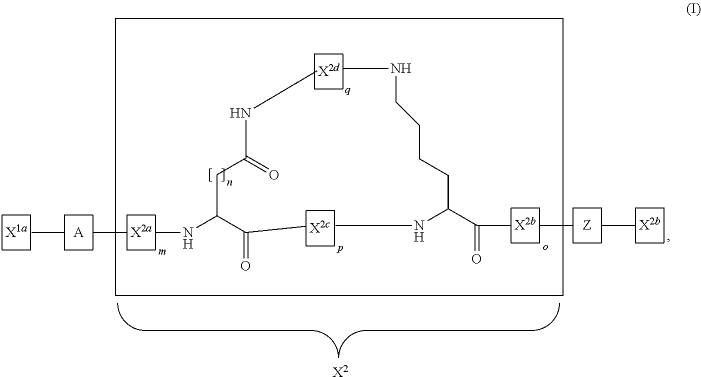

wherein X^{1a}, A, Z, and X^{1b} are as described herein. In formula (I) above, X^{2a} and X^{2b} are amino acids of X², wherein X^{2a} and X^{2b} may be independently selected from any amino acid; X²⁰ and X^{2d} comprise amino acids which provide a cleavable linker (e.g., an enzymatically cleavable linker); n is one or two; m and o are at least one, and may be independently selected from an integer ranging from 1 to 30; p and q are each at least two, and may be independently selected from an integer ranging from two to thirty, wherein the cleavage sites provided by X²⁰ and X^{2d} may the same or different, may be susceptible to cleavage by the same or different conditions (e.g., the same or different enzymes), and may be, for example, independently selected from any of the cleavable linkers described herein. Where X²⁰ and X^{2d} define cleavable linkers that are each susceptible to cleavage under different conditions (e.g., different enzymes), the molecule can be described as comprising a chimeric linker. Such combinations may include enzymes of the same or different class.

Synthesis of a molecule of formula (I) can be performed with standard peptide coupling chemistry. For example, as a precursor to compounds of formula (I), standard peptide coupling chemistry can be used to make a compound of the formula (II) below, wherein X² comprises an aspartic acid or glutamic acid and lysine residues.

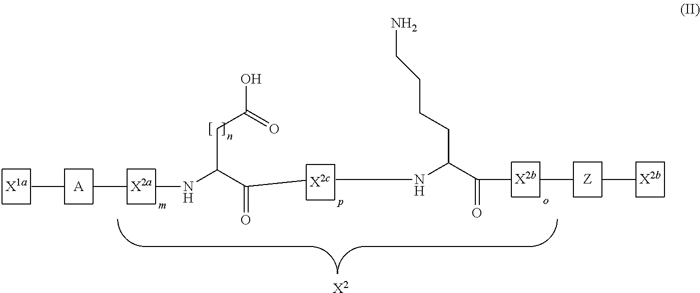

A peptide coupling reaction typically employs a conventional peptide coupling reagent and is conducted under conventional coupling reaction conditions, typically in the presence of a trialkylamine, such as ethyldiisopropylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF.

During any of the processes for preparation of the compounds, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known in the art. For example, the aspartic acid, glutamic acid, and lysine residues can be protected with various protecting groups during the synthetic process. Depending on the type of protecting group used, selectivity in deprotection can be used advantageously during the synthetic process. One of ordinary skill in the art would be able to select the type of protecting group that is appropriate for the synthetic scheme.

The dual linker described above having a cyclic structure can be synthesized by using the carboxyl side chain of aspartic acid or glutamic acid as the carboxyl handle of a peptide backbone and amino side chain of lysine as the amino handle of a peptide backbone. Synthesis of formula (I) can be performed using standard peptide coupling chemistry. Peptide coupling reactions typically employ a conventional peptide coupling reagent and are conducted under conventional coupling reaction conditions as discussed above.

Portions X^{1A} and X^{1B}

Promolecules of the present disclosure may include optional portions X^{1a} and X^{1b} that, when present, comprise a nucleophilic moiety and facilitate the attachment of one or more cargo moieties to the promolecule. The nucleophilic moiety of portions X^{1a} and X^{1b} generally comprises a nucleophilic reactive group comprising at least one pair of free electrons that is capable of reacting with an electrophile. Examples of nucleophilic moieties include sulfur nucleophiles, such as thiols, thiolate anions, anions of thiolcarboxylate, anions of dithiocarbonates, and anions of dithiocarbamates; oxygen nucleophiles, such as hydroxide anion, alcohols, alkoxide anions, and carboxylate anions; nitrogen nucleophiles, such as amines, azides, and nitrates; and carbon nucleophiles, such as alkyl metal halides and enols.

Cargo moieties may be, e.g., detectable moieties that can facilitate detection of a promolecule through various imaging modalities, or may be, e.g., therapeutic agents that can facilitate treatment of a disease or condition.

Non-limiting examples of detectable moieties include fluorescent dyes and radioisotopes. In some embodiments, two or more cargo moieties may be attached to the same promolecule (e.g., a fluorescent dye and a radioisotope attached to the same promolecule). Differing cargo moieties may be paired for simultaneous detection using multiple modalities. For example, non-invasive detection using nuclear imaging agents could be coupled with fluorescence to enable follow on studies for enhanced yet invasive (e.g., surgical) detection.

In some embodiments, a detectable moiety may comprise a fluorescent dye. Non-limiting examples of fluorescent dyes that may be conjugated to promolecules of the present disclosure include cyanine dyes, such as fluorescein, tetramethoxyrhodamine, Cy2, Cy3, Cy3B, Cy3.5, Cy5, Cy5.5, or Cy7, IRdye 800cw, or ATTO-TEC™ dyes, such as ATTO 680. Suitable cargo moieties also include fluorescent dyes having longer wavelengths in the near-infrared region. Such dyes are known in the art and can be readily incorporated into the compounds of the present disclosure.

In some embodiments, a detectable moiety may comprise a metal chelating moiety. Non-limiting examples of metal chelating moieties include 1,4,7,10-tetraazacyclododecane-1,4,7,10-tetraacetic acid (DOTA), 1,4,7-Triazacyclononane-1,4,7-triacetic acid (NOTA), 1,4-bis(carboxymethyl)-6-[bis(carboxymethyl)]amino-6-methyl-perhydro-1,4-diazepine (AAZTA), desferrioxamine (DFO), 3,4,3-(LI-1,2-HOPO) (HOPO), diethylenetriaminepentaacetic acid (DTPA), 4,11-bis-(carboxymethyl)-1,4,8,11-tetraazabicyclo[6.6.2]-hexadecane (CB-TE2A), N,N′-bis(2-hydroxybenzyl)-ethylenediamine-N,N′-diacetic acid (HBED).

In some embodiments, a detectable moiety may comprise a radioisotope, e.g., a radioisotope chelated through a metal binding moiety. Non-limiting examples of radioisotopes include Actinium-225, Astatine-211, Bismuth-212, Bismuth-213, Bromine-76, Bromine-77, Calcium-47, Carbon-11, Carbon-14, Chromium-51, Cobalt-57, Cobalt-58, Copper-64, Erbium-169, Fluorine-18, Gallium-67, Gallium-68, Hydrogen-3, Indium-111, Iodine-123, Iodine-125, Iodine-131, Iron-59, Krypton-81m, Lead-212, Lutetium-177, Nitrogen-13, Oxygen-15, Phosphorus-32, Radium-223, Radium-224, Samarium-153, Selenium-75, Sodium-22, Sodium-24, Strontium-89, Technetium-99m, Thallium-201, Thorium-226, Thorium-227, Xenon-133, or Yttrium-9. In some embodiments, the detectable moiety is Copper-64.

In some embodiments, the detectable moiety is the radioisotope Copper-64 conjugated to the metal chelating moiety DOTA.

In some embodiments, the cargo moiety is a radioisotope. In such embodiments, the cargo moiety may be detected using X-rays, fluoroscopy, angiography, positron emission tomography (PET), or single positron emission computed tomography (SPECT) wherein cells, tissues, or entire subjects are placed in the field of the imaging modality and visualized.

In some embodiments, detection of the cleavage products is performed at the organismal level. In some embodiments, the detection is performed within a target region. In some embodiments, the detection occurs at extended time points post-administration (e.g., post-injection, such as post-intravenous administration), such as from 0.5 to 24 hours post-injection. In some embodiments, the detection occurs at multiple time points post-administration, e.g., post-injection.

In some embodiments, a single promolecule having features of the present disclosure may include more than one cargo moiety so that portion A may be linked to multiple detectable moieties, or to both a detectable moiety and a therapeutic agent, or to multiple therapeutic agents. Such multiple detectable moieties may include different types of markers, and may allow, for example, attachment of both a radioisotope and a contrast agent or fluorescent dye, allowing imaging by different modalities.

Promolecules comprising a detectable moiety conjugated through portion X^{1a} or X^{1b} may have use in visualization or identification of cells having a certain condition or cells in a region exhibiting a particular condition. For example, granzyme activity or activation of immune cells that secrete granzyme may be visualized by designing an X² linker to be cleaved by a granzyme of interest, such as granzyme B, so that a cleavage product comprising portion A interacts with cell membranes in the vicinity of the granzyme activity or immune cell activation. The interaction of portion A with cell membranes delivers a radioisotope or other marker to the region. Thus, radioisotopes are one example of a cargo moiety that may be delivered to target cell membranes or phospholipid bilayer structures in specific regions upon cleavage of X².

Non-limiting examples of therapeutic agents that can be conjugated to the promolecules of the present disclosure include radioisotopes such as Actinium-225, Astatine-211, Bismuth-212, Bismuth-213, Bromine-76, Bromine-77, Calcium-47, Carbon-11, Carbon-14, Chromium-51, Cobalt-57, Cobalt-58, Copper-64, Erbium-169, Fluorine-18, Gallium-67, Gallium-68, Hydrogen-3, Indium-111, Iodine-123, Iodine-125, Iodine-131, Iron-59, Krypton-81m, Lead-212, Lutetium-177, Nitrogen-13, Oxygen-15, Phosphorus-32, Radium-223, Radium-224, Samarium-153, Selenium-75, Sodium-22, Sodium-24, Strontium-89, Technetium-99m, Thallium-201, Thorium-226, Thorium-227, Xenon-133, or Yttrium-9.

In some embodiments, a particular moiety may function as both a detectable moiety and as a therapeutic agent.

### Methods of Making

Promolecules of the present disclosure can be made by any suitable method, including but not limited to recombinant and non-recombinant (e.g., chemical synthesis) methods. Cargo moieties may be conjugated to promolecules by any suitable method, including but not limited to nucleophilic addition reactions.

### Production of Promolecules

The promolecules of the present disclosure can be produced by any suitable method, including recombinant and non-recombinant methods (e.g., chemical synthesis).

Where a polypeptide is chemically synthesized, the synthesis may proceed via liquid-phase or solid-phase. Solid-phase synthesis (SPPS) allows the incorporation of unnatural amino acids, peptide/protein backbone modification. Various forms of SPPS, such as Fmoc and Boc, are available for synthesizing peptides of the present disclosure. Details of the chemical synthesis are known in the art (e.g., Ganesan A. 2006 Mini Rev. Med Chem. 6:3-10 and Camarero J A et al. 2005 Protein Pept Lett. 12:723-8). Briefly, small insoluble, porous beads are treated with functional units on which peptide chains are built. After repeated cycling of coupling/deprotection, the free N-terminal amine of a solid-phase attached peptide or amino acid is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Where the polypeptide is produced using recombinant techniques, the proteins may be produced as an intracellular protein or as a secreted protein, using any suitable construct and any suitable host cell, which can be a prokaryotic or eukaryotic cell, such as a bacterial (e.g. E. coli) or a yeast host cell, respectively.

Other examples of eukaryotic cells that may be used as host cells include insect cells, mammalian cells, and/or plant cells. Where mammalian host cells are used, the cells may include one or more of the following: human cells (e.g. HeLa, 293, H9 and Jurkat cells); mouse cells (e.g., X3, NIH3T3, pancreatic ductal adenocarcinoma 2.1, L cells, and C127 cells); primate cells (e.g. Cos 1, Cos 7 and CV1) and hamster cells (e.g., Chinese hamster ovary (CHO) cells).

A wide range of host-vector systems suitable for the expression of the subject polypeptide may be employed according to standard procedures known in the art. See, e.g., Sambrook et al. 1989 Current Protocols in Molecular Biology Cold Spring Harbor Press, New York and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are available commercially.

Vectors can provide for extrachromosomal maintenance in a host cell or can provide for integration into the host cell genome. The expression vector provides transcriptional and translational regulatory sequences, and may provide for inducible or constitutive expression, where the coding region is operably linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like). Expression constructs generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selectable genes are well known in the art and will vary with the host cell used.

Isolation and purification of a protein and/or antibody can be accomplished according to methods known in the art. For example, a protein can be isolated from a lysate of cells genetically modified to express the protein constitutively and/or upon induction, or from a synthetic reaction mixture, by immunoaffinity purification, which generally involves contacting the sample with an anti-protein antibody, washing to remove non-specifically bound material, and eluting the specifically bound protein. The isolated protein can be further purified by dialysis and other methods normally employed in protein purification methods. In one embodiment, the protein may be isolated using metal chelate chromatography methods. Protein of the present disclosure may contain modifications to facilitate isolation.

The subject polypeptides may be prepared in substantially pure or isolated form (e.g., free from other polypeptides). The protein can be present in a composition that is enriched for the polypeptide relative to other components that may be present (e.g., other polypeptides or other host cell components). Purified protein may be provided such that the protein is present in a composition that is substantially free of other expressed proteins, e.g., less than 98%, less than 95%, less than 90%, less than 80%, less than 60%, or less than 50%, of the composition is made up of other expressed proteins.

### Conjugation of Cargo Moieties to Polypeptides

Cargo moieties may be conjugated to promolecules of the present disclosure using any suitable technique, including but not limited to nucleophilic addition reactions that utilize nucleophilic moieties. Non-limiting examples of such reactions include reactions of sulfur nucleophiles, oxygen nucleophiles, carbon nucleophiles, or nitrogen nucleophiles with a suitable electrophile to form a covalent bond.

### Optional Modifications

Promolecules of the present disclosure may be further modified to generally provide, e.g., longer circulating half-life, restriction of the promolecules to certain anatomical compartments (e.g., restriction to the cardiovascular system), protection against non-specific degradation, and/or enhanced sensitivity to certain imaging modalities.

In some embodiments, two or more promolecules may be linked to a central molecule, e.g., a polyethylene glycol (PEG) molecule, to form a dendrimer using techniques that are known in the art. Suitable PEG molecules may have a molecular weight of up to about 1,000, up to about 5,000, up to about 10,000, up to about 20,000, up to about 30,000, or up to about 40,000 Daltons. Conjugation of two or more promolecules to a central PEG molecule can be accomplished by, e.g., activating a PEG molecule with a functional group at one or more termini and then reacting the activated PEG molecule with one or more promolecules of the present disclosure. The choice of functional groups depends on the available reactive groups on the promolecule, such as the N-terminal amine, the C-terminal carboxylic acid, or residues such as lysine, aspartic acid, cysteine, glutamic acid, serine, threonine, or other specific reactive sites. Linear, single-arm PEG structures, as well as branched PEG structures may be created using such techniques.

In some embodiments, a linear, single-arm PEG structure is formed having the general formula: X^{1a}-A-X²—Z—X³, where X³ is a PEG molecule and X^{1a}, A, X², and Z are as described above. In other embodiments, a branched PEG dendrimer is formed using techniques known in the art, wherein two or more promolecules of the present disclosure are conjugated to the branched PEG dendrimer to form a polyvalent PEG structure.

### Compositions

Compositions comprising any of the promolecules of the present disclosure are also provided. The compositions may include any of the promolecules of the present disclosure, including any of the promolecules described hereinabove and in the Experimental section below.

In certain embodiments, a composition of the present disclosure comprises any of promolecules of the present disclosure, present in a liquid medium. The liquid medium may be an aqueous liquid medium, such as water, a buffered solution, or the like. One or more additives such as a salt (e.g., NaCl, MgCl2, KCl, MgSO4), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. The promolecules of the present disclosure can be formulated in a variety of pharmaceutical compositions suitable for administration to a subject (e.g., by a desired route). A composition comprising a promolecule of the present disclosure may comprise a pharmaceutically acceptable excipient, a variety of which are known in the art and need not be discussed in detail herein.

In some embodiments, promolecules of the present disclosure are formulated for parenteral administration to a subject, e.g., intravenous administration. Pharmaceutically acceptable excipients have been amply described in a variety of publications, including, for example, “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

In some cases, a subject pharmaceutical composition will be suitable for injection into a subject, e.g., will be sterile. For example, in some embodiments, a subject pharmaceutical composition will be suitable for injection into a human subject, e.g., where the composition is sterile and is free of detectable pyrogens and/or other toxins.

A subject pharmaceutical composition may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH-adjusting and buffering agents, tonicity-adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

Promolecules of the present disclosure may be formulated into unit dosage forms that contain a predetermined amount of the promolecules disclosed herein. Unit dosage forms suitable for injection or intravenous administration may comprise promolecules of the present disclosure in a composition as a solution in sterile water, normal saline, or another pharmaceutically acceptable carrier.

The term “unit dosage form,” as used herein, refers to physically discrete units suitable as unitary dosages for human and animal subjects, each unit containing a predetermined quantity of promolecules of the present disclosure calculated in an amount sufficient to produce the desired effect in association with a pharmaceutically acceptable diluent, carrier or vehicle. The specifications for the unit dosage forms of the present disclosure depend on the particular promolecule employed and the effect to be achieved, and the pharmacodynamics associated with each promolecule in the subject.

Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Promolecules of the present disclosure may also be formulated for oral administration to a patient. For oral preparations, promolecules can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The promolecules of the present disclosure may be utilized in aerosol formulations to be administered via inhalation, or may be formulated into acceptable pressurized propellants, such as dichlorodifluoromethane, propane, nitrogen, and the like.

Furthermore, promolecules of the present disclosure can be made into suppositories by mixing with a variety of bases such as emulsifying bases or water-soluble bases. The promolecules of the present invention can be administered rectally via a suppository. The suppository can include vehicles such as cocoa butter, carbowaxes, and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the promolecules of the present disclosure. Similarly, unit dosage forms for injection or intravenous administration may comprise one or more promolecules in a composition as a solution in sterile water, normal saline or another pharmaceutically acceptable carrier.

In some embodiments, promolecules of the present disclosure are formulated for local administration to a subject, e.g., at or near a site of desired action. In some embodiments, promolecules of the present disclosure are formulated in a sustained release dosage form that is designed to release promolecules at a predetermined rate for a specific period of time.

Promolecules of the present disclosure may also be formulated with agents that influence the pharmacokinetic profile of the promolecule when administered to a subject. Such agents include verapamil or other equivalents.

### Routes of Administration

In practicing the methods of the present disclosure, routes of administration may be selected according to any of a variety of factors, such as properties of the promolecule to be delivered, the type of condition being diagnosed, detected, or treated (e.g., detection of clotting), and the like. Promolecules of the present disclosure may be delivered by a route of administration that provides delivery of the promolecule to the bloodstream (e.g., by parenteral administration, such as intravenous administration, intramuscular administration, and/or subcutaneous administration) or to a specific tissue or organ (e.g., muscle tissue, cardiac tissue, vascular tissue, and the like). Injection can be used to accomplish parenteral administration. In some embodiments, promolecules are delivered by a route of administration that provides for delivery of the promolecule directly into affected tissue, e.g., by direct injection into the target tissue or organ.

Promolecules of the present disclosure may be administered through the respiratory tract. Such dosage forms may be smoking devices, dry powder inhalers, pressurized metered dose inhalers, nebulizers, vaporizers, or the like.

Promolecules of the present disclosure may be administered orally by having the subject swallow a suitable dosage form, such as tablets, powders, granules, capsules, elixirs, syrups, or the like. Promolecules of the present disclosure may also be administered rectally in the form of suppositories.

Promolecules of the present disclosure may be administered by direct injection into a target tissue or into the blood stream, including intradermal, subcutaneous, intravenous, intracardiac, intramuscular, intraosseous, or intraperitoneal injection. Promolecules of the present disclosure can be administered by intracavernous or intravitreal delivery to organs or tissues, or administered by intracerebral, intrathecal, or epidural delivery to tissues of the central nervous system.

Promolecules of the present disclosure may be administered locally or topically. Such administration may be accomplished by topically applying a suitable formulation directly to a target tissue. The previously-described routes of administration, formulations and dosage forms are merely exemplary and are in no way limiting.

### Dosages

In the methods of the present disclosure, an amount of a promolecule that is effective to achieve the desired diagnosis, detection, or treatment is administered to a subject.

The amount administered varies depending upon the goal of the administration, the health and physical condition of the individual to be treated, age, the degree of resolution desired, the formulation of a subject composition, the activity of the subject composition employed, the treating clinician's assessment of the medical situation, the condition of the subject, the body weight of the subject, as well as the severity of the disease, disorder, or condition being diagnosed, detected, and/or treated, and other relevant factors. The size of the dose will also be determined by the existence, nature, and extent of any adverse side-effects that might accompany the administration of a particular composition.

It is expected that the amount will fall in a relatively broad range that can be determined through routine trials. For example, the amount of a promolecule of the present disclosure employed to detect granzyme activity or activation of granzyme-secreting immune cells in a subject is not more than about the amount that could otherwise be irreversibly toxic to the subject (i.e., maximum tolerated dose). In other cases, the amount is around or even well below the toxic threshold, but still in an effective concentration range, or even as low as a threshold dose. In some embodiments, a dose of from 1 to 200 μg, 50 to 150 μg, or 75 to 125 μg (e.g., about 100 μg) is administered to a subject to detect granzyme activation or the activation of granzyme-secreting immune cells.

In certain embodiments, the promolecule is administered intravenously and within the mass dose limitations defined by the FDA for a PET microdosing study. According to some embodiments, the limitations are 1/100th of the minimal pharmacologically active dose or less than 100 μg. In some instances, a dose applied to humans is dictated by animal (e.g., mouse) dosimetry data. In certain embodiments, the promolecule is administered at a dose of from 1 mCi/injection to 20 mCi/injection, e.g., from 5 mCi/injection to 15 mCi/injection. An effective amount of the promolecule may be administered in one or more administrations, e.g., one or more, two or more, three or more, four or more, or five or more administrations.

### Methods of Use

The present disclosure provides methods of using activatable and detectable membrane-interacting peptides for the diagnosis and/or treatment of diseases or conditions generally involving localized biological processes, such as proteolysis. For example, in certain cases, the promolecules of the present disclosure find use as a diagnostic tool in guiding and/or monitoring therapy (e.g., an immunomodulatory therapy, such as a cell-based therapy, e.g., CAR T cell therapy or the like). Such methods generally involve detection of biological processes, such as proteolysis, which may be associated with a particular disease, condition and/or immune response. The promolecules of the present disclosure also find use in treating particular diseases or conditions, and in methods that involve delivery of therapeutic agents to a particular site or location within a patient.

In general, the methods of the present disclosure involve selecting a promolecule that contains a cleavable linker X² that is cleaved by granzyme under conditions associated with a disease or condition to be diagnosed, detected, or treated, and administering the promolecule to a subject in an amount that is sufficient to facilitate detection of the cleavage-promoting condition(s) or to facilitate treatment of a disease/condition associated with the cleavage-promoting condition(s). Administering can be by any suitable route, and the promolecule can be selected according to the disease or condition to be diagnosed or treated. For example, in the case of detection of granzyme activity or activation of granzyme-secreting immune cells within a subject, administration can be intravenous.

When the promolecule encounters granzyme cleavage-promoting conditions (e.g., the release of granzyme by activated granzyme-secreting immune cells) within the subject, the promolecule is cleaved by granzyme and the cleavage product containing the membrane-interacting peptide inserts into and detectably labels membranes in the vicinity of the cleavage-promoting environment. Detection of this cleavage product identifies the presence of granzyme activity in the labeled region. In diagnostic uses and methods, identification of the region within the subject where granzyme cleavage promoting conditions exist facilitates diagnosis of a disease or condition, and may then provide guidance for administering and/or monitoring an appropriate therapy. In therapeutic uses and methods, delivery of a therapeutic agent to a targeted site within the patient facilitates treatment of a disease or condition.

According to some embodiments, the present disclosure provides methods of detectably labeling a phospholipid bilayer of a cell in the presence of granzyme activity. Such methods comprise contacting a promolecule of the present disclosure with granzyme contributing to the granzyme activity, where the cleavable linker of the molecule is cleaved by the granzyme to release a cleavage product comprising a detectable moiety and a membrane interacting polypeptide portion, such that the membrane interacting polypeptide portion interacts with the phospholipid bilayer of the cell and detectably labels the phospholipid bilayer of the cell in the presence of granzyme activity. In some embodiments, the contacting is in vitro, in vivo, or ex vivo.

According to some embodiments, the present disclosure provides methods of assessing granzyme activity in a cellular sample. Such methods comprise contacting the sample with a promolecule of the present disclosure, wherein in the presence of granzyme activity, the promolecule is cleaved to release a cleavage product comprising the detectable moiety and the membrane interacting polypeptide portion, and wherein the cleavage product interacts with a phospholipid bilayer of a cell in the presence of the granzyme activity. Such methods further comprise assessing for the presence or absence of the detectable moiety of the cleavage product, wherein the presence of the detectable moiety indicates granzyme activity in the cellular sample.

### Diagnostic Uses and Methods

The methods of the present disclosure generally relate to diagnosis and detection of diseases or conditions that involve localized biological processes, such as proteolysis. In some embodiments, the methods of the present disclosure relate to detecting proteolysis resulting from the activity of one or more granzymes that are associated with a particular condition. Such granzymes may be, e.g., bound to or associated with cells located in particular tissues or organs, and the identification of such cells may be useful in guiding and/or monitoring therapy. For example, the enzyme granzyme is associated with the activation of granzyme-secreting immune cells, and the identification of such sites may be useful in determining sites of immune cell activation following immunomodulatory therapy.

The methods of the present disclosure can be adapted to provide for methods of monitoring therapy. For example, a promolecule of the present disclosure can be administered to a subject prior to, during (e.g., between doses), and/or after therapy, and the signal associated with the promolecule can be detected to facilitate the effect of therapy upon the condition being treated. Non-limiting example methods of the present disclosure are provided below.

### Assessing Granzyme Activity in a Subject

According to some embodiments, the present disclosure provides methods of assessing granzyme activity in a subject. Such methods comprise administering the promolecule of the present disclosure, wherein at sites of granzyme activity in the subject, the promolecule is cleaved by a granzyme contributing to the granzyme activity to release cleavage products comprising the detectable moiety and the membrane interacting polypeptide portion, and wherein the cleavage products interact with the phospholipid bilayers of cells at the sites of granzyme activity in the subject. Such methods further comprise assessing for the presence or absence of cells labeled with the cleavage products, wherein the presence of cells labeled with the cleavage products indicates granzyme activity in a subject.

In some embodiments, promolecules are administered to a subject to assess granzyme activity in the subject. For example, a promolecule having a granzyme-cleavable X² linker and having a radioisotope moiety conjugated thereto is administered intravenously to a subject. When the promolecule comes into contact with the granzyme at a site of granzyme activity in the subject, X² is cleaved, forming cleavage products containing portions A and Z. The membrane-interacting peptide of portion A then undergoes a conformational change to form an alpha-helical structure that inserts into cell membranes in the area of the granzyme activity. In some embodiments, the cargo moiety attached to the promolecule is a radioisotope. Once the cleavage product containing portion A has inserted into the plasma membranes of cells in the area of granzyme activity, as described above, the radioisotope is detected using an appropriate imaging modality, e.g., fluoroscopy, X-rays, single photon emission computed tomography (SPECT), magnetic resonance (MR) or positron emission tomography (PET) to identify tissues in the subject in which granzyme activity is taking place. In some embodiments, assessing granzyme activity in a subject comprises identifying regions in the subject in which granzyme activity occurs.

### Assessing Granzyme-Secreting Immune Cell Activation in a Subject

According to some embodiments, the present disclosure provides method of assessing for activation of immune cells in a subject, wherein the immune cells secrete granzyme upon activation in the subject. Such methods comprise administering the promolecule of the present disclosure to the subject, wherein at sites of activated immune cell-secreted granzyme in the subject, the molecule is cleaved by the activated immune cell-secreted granzyme to release cleavage products comprising the detectable moiety and the membrane interacting polypeptide portion, and wherein the cleavage products interact with the phospholipid bilayers of cells at the sites of activated immune cell-secreted granzyme in the subject. Such methods further comprise assessing for the presence or absence of cells labeled with the cleavage products, wherein the presence of cells labeled with the cleavage products indicates activation of the immune cells in the subject.

In certain embodiments, promolecules are administered to a subject to assess for granzyme-secreting immune cell activation. In some embodiments, the granzyme-secreting immune cells are NK cells or CTLs. In some embodiments, the granzyme secreted by the CTLs is granzyme B or granzyme K.

For example, a promolecule having a granzyme-cleavable X² linker and having a radioisotope moiety conjugated thereto is administered intravenously to a subject. When the promolecule comes into contact with granzyme at a site of granzyme secretion by activated immune cells, X² is cleaved, forming cleavage products containing portions A and Z. The membrane-interacting peptide of portion A then undergoes a conformational change to form an alpha-helical structure that inserts into cell membranes in the area of granzyme-secreting immune cell activation.

According to some embodiments, the cargo moiety attached to the promolecule is a radioisotope. Once the cleavage product containing portion A has inserted into the plasma membranes of cells in the area of granzyme activity, as described above, the radioisotope is detected using an appropriate imaging modality, e.g., fluoroscopy, X-rays, single photon emission computed tomography (SPECT), magnetic resonance (MR) or positron emission tomography (PET) to distinguish the locations and/or tissues in the subject in which granzyme-secreting immune cell activation is taking place.

In certain embodiments, assessing for granzyme-secreting immune cell activation is performed at the organismal level. In some embodiments, the assessing is performed within a target region. In some embodiments, the detection occurs at extended time points post-administration (e.g., post-injection, such as post-intravenous administration), such as from 0.5 to 24 hours post-injection. In some embodiments, the detection occurs at multiple time points post-administration, e.g., post-injection.

According to some embodiments, assessing for granzyme-secreting immune cell activation comprises assessing for immune cells activated as part of an immune response to a pathogen. In certain embodiments, the pathogen is a microorganism that can cause disease in a subject. According to some embodiments, the subject has, or is suspected of having, a pathogen infection.

In some embodiments, assessing for granzyme-secreting immune cell activation comprises assessing for immune cells activated as part of an immune response to a viral infection. Examples of such viral infections include, but are not limited to, an infection by Adenoviridae (e.g., adenovirus), Arenaviridae (e.g., Machupo virus), Bunyaviridae (e.g., Hantavirus or Rift Valley fever virus), Coronaviridae, Orthomyxoviridae (e.g., influenza viruses), Filoviridae (e.g., Ebola virus and Marburg virus), Flaviviridae (e.g., Japanese encephalitis virus and Yellow fever virus), Hepadnaviridae (e.g., hepatitis B virus), Herpesviridae (e.g., herpes simplex viruses), Papovaviridae (e.g., papilloma viruses), Paramyxoviridae (e.g., respiratory syncytial virus, measles virus, mumps virus, or parainfluenza virus), Parvoviridae, Picornaviridae (e.g., polioviruses), Poxviridae (e.g., variola viruses), Reoviridae (e.g., rotaviruses), Retroviridae (e.g., human T cell lymphotropic viruses (HTLV) and human immunodeficiency viruses (HIV)), Rhabdoviridae (e.g., rabies virus), and Togaviridae (e.g., encephalitis viruses, yellow fever virus, and rubella virus). In certain embodiments, the virus causes pneumonia.

According to some embodiments, assessing for granzyme-secreting immune cell activation comprises assessing for immune cells activated as part of an immune response to a bacterial infection. Examples of such bacterial infections include, but are not limited to, an infection by Bacillus (e.g., B. anthracis), Enterobacteriaceae (e.g., Salmonella, Escherichia coli, Yersinia pestis, Klebsiella, and Shigella), Yersinia (e.g., E. pestis or E. enterocolitica), Staphylococcus (e.g., S. aureus), Streptococcus, Gonorrheae, Enterococcus (e.g., E. faecalis), Listeria (e.g., L. monocytogenes), Brucella (e.g., B. abortus, B. melitensis, or B. suis), Vibrio (e.g., V. cholerae), Corynebacterium diphtheria, Pseudomonas (e.g., P. pseudomallei or P. aeruginosa), Burkholderia (e.g., B. mallei or B. pseudomallei), Shigella (e.g., S. dysenteriae), Rickettsia (e.g., R. rickettsii, R. prowazekii, or R. typhi), Francisella tularensis, Chlamydia psittaci, Coxiella burnetii, and Mycoplasma (e.g., M. mycoides). In certain embodiments, the bacterium is Escherichia coli (E. coli), Staphylococcus aureus (S. aureus), Pseudomonas aeruginosa (P. aeruginosa), or Klebsiella pneumoniae (K. pneumoniae). According to some embodiments, the bacterium is E. coli. In certain embodiments, the bacterium is S. aureus. In certain embodiments, the bacterium is P. aeruginosa. According to some embodiments, the bacterium is K. pneumoniae.

In some embodiments, assessing for granzyme-secreting immune cell activation comprises assessing for the presence or absence of T-cell exhaustion in the subject. T-cell exhaustion is characterized by the deterioration and loss of T-cell functions (e.g., granzyme secretion), culminating in the loss of the T-cells. For example, promolecules of the present disclosure may be administered to the subject at one or more timepoints (e.g., one or more, two or more, three for more, four or more, or five or more timepoints) in order to identify the presence or absence of T-cell exhaustion by assessing for changes in the activation of granzyme-secreting immune cells over time. As described above, a promolecule having a cleavable X² linker that is cleaved by granzyme and having a fluorescent cargo moiety or a radioisotope cargo moiety conjugated thereto is administered intravenously to the subject. If granzyme-secreting immune cell activation is present, the promolecule will be cleaved by granzyme, portion A will undergo a conformational change to form an alpha-helical structure and insert into cell membranes in the vicinity of the granzyme-secreting immune cell activation, and the location of activation of granzyme-secreting immune cells can be detected by visualizing the fluorescent moiety or radioisotope. Following administration of the promolecules at multiple time points and visualization of granzyme-secreting immune cell activation, changes in the extent of granzyme-secreting immune cell activation over time can be assessed qualitatively and/or quantitatively. If the activation of granzyme-secreting immune cells is not present or has decreased, then no signal or a diminished signal, respectively, will be detected. Decreases in the extent of granzyme-secreting immune cell activation indicate decreased T-cell function and the presence of T-cell exhaustion.

In some embodiments, assessing for granzyme-secreting immune cell activation comprises assessing for an immune response in the subject. In a further embodiment, assessing for an immune response comprises assessing whether the subject is at risk for developing immune related adverse effects. For example, promolecules of the present disclosure are administered to the subject in order to determine the locations of granzyme-secreting immune cell activation. As described above, a promolecule having a cleavable X² linker that is cleaved by granzyme and having a fluorescent cargo moiety or a radioisotope cargo moiety conjugated thereto is administered intravenously to the subject. If granzyme-secreted immune cell activation is present, the promolecule will be cleaved by granzyme, portion A will undergo a conformational change to form an alpha-helical structure and insert into cell membranes in the region of granzyme-secreting immune cell activation, and regions of granzyme-secreting immune cell activation can be detected by visualizing the fluorescent moiety or radioisotope. Detection of systemic granzyme-secreting immune cell activation in normal (e.g., non-tumor) tissues according to the methods of the present disclosure may serve to identify the subject as being at risk for developing immune related adverse effects.

### Monitoring Therapy in a Subject

In some embodiments, promolecules of the present disclosure are administered to a subject during or after therapy in order to monitor the progress of the therapy. For example, after an immunomodulatory therapy has been performed on a subject, promolecules of the present disclosure may be administered to the subject in order to determine whether activation of granzyme-secreting immune cells has occurred and the locations of said activated immune cells. As described above, a promolecule having a cleavable X² linker that is cleaved by granzyme and having a fluorescent cargo moiety or a radioisotope cargo moiety conjugated thereto is administered intravenously to the subject. If there is granzyme-secreting immune cell activation, the promolecule will be cleaved by granzyme at the site of immune cell activation, portion A will undergo a conformational change to form an alpha-helical structure and insert into cell membranes in the vicinity, and the activation of granzyme-secreting immune cells can be detected by visualizing the fluorescent moiety or radioisotope. If the activation of granzyme-secreting immune cells is not present or has decreased, then no signal or a diminished signal, respectively, will be detected at the site of treatment. This information can then be used by the treating physician to monitor the progress of the therapeutic efforts.

In some embodiments, monitoring the progress of the therapy comprises monitoring the progress of an immunomodulatory therapy in a subject. In some embodiments, the immunomodulatory therapy comprises a cell-based therapy (that is, the transfer of autologous or allogeneic cellular material into the subject for medical purposes), stimulator of interferon genes (STING) pathway modulation, immune checkpoint inhibition, chemotherapy, ionizing radiation, or any combination thereof. In some embodiments, the immunomodulatory therapy comprises a cell-based therapy. In some embodiments, the cell-based therapy comprises chimeric antigen receptor T-cell (CAR-T) therapy, chimeric antigen receptor NK cell (CAR-NK) therapy, or the administration of T-cells comprising an engineered T-cell receptor. In some embodiments, the immunomodulatory therapy is used to treat cancer in a subject.

In certain embodiments, the cell-based therapy comprises administering cells (e.g., T cells, NK cells, or the like) engineered to express a receptor (e.g., a chimeric antigen receptor (CAR) or a T cell receptor (TCR) such as a recombinant TCR). According to some embodiments, when the cells are engineered to express a receptor on its surface, the extracellular binding domain of the receptor specifically binds a tumor antigen expressed on the surface of a cancer cell. Non-limiting examples of tumor antigens to which the extracellular binding domain of the receptor may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), Wilms' tumor 1 (WT1), and VEGF-A.

In some embodiments, monitoring the progress of an immunomodulatory therapy for treating cancer comprises assessing a tumor in the subject for responsiveness to the immunomodulatory therapy. For example, while a subject is undergoing immunomodulatory therapy for cancer, promolecules of the present disclosure are administered to the subject in order to determine whether activation of granzyme-secreting immune cells has occurred in the vicinity of the tumor. As described above, a promolecule having a cleavable X² linker that is cleaved by granzyme and having a fluorescent cargo moiety or a radioisotope cargo moiety conjugated thereto is administered intravenously to the subject. If granzyme-secreted immune cell activation is present in the vicinity of the tumor, the promolecule will be cleaved by granzyme, portion A will undergo a conformational change to form an alpha-helical structure and insert into cell membranes in the vicinity of the tumor, and the location of activation of granzyme-secreting immune cells in the vicinity of the tumor can be detected by visualizing the fluorescent moiety or radioisotope.

In some embodiments, monitoring the progress of immunomodulatory therapy comprises assessing for the presence or absence of T-cell exhaustion in a subject as described above. For example, while the subject is undergoing immunomodulatory therapy comprising the administration of therapeutic T cells to the subject, promolecules of the present disclosure may be administered to the subject at one or multiple timepoints in order to assess for T-cell exhaustion (e.g., identify the presence or absence, or extent, of T-cell exhaustion) by assessing for activation of granzyme-secreting therapeutic T cells at the one or multiple timepoints. Decreases in the extent of granzyme-secreting immune cell activation indicate decreased T-cell function and the presence of T-cell exhaustion.

In some embodiments, monitoring the progress of immunomodulatory therapy comprises monitoring for an immune response in the subject as described above. In a further embodiment, monitoring for an immune response comprises assessing whether the subject is at risk for developing immune related adverse effects as described above. For example, promolecules of the present disclosure are administered to the subject in order to determine the locations of granzyme-secreting immune cell activation, as described above. Detection of systemic granzyme-secreting immune cell activation in normal tissues (e.g., non-tumor tissue) indicates that the subject is at risk for developing immune related adverse effects.

### Methods of Detecting Cleavage Products a and Z in Diagnostic Applications

Cleavage products A and Z may be detected through a variety of imaging and detection modalities, including by not limited to fluorescence microscopy, X-rays, fluoroscopy, angiography, positron emission tomography (PET), and the like. Detection can be accomplished by directly imaging the cells or tissues in which the cleavage product is located, or by contacting the cells or tissues in which the cleavage product is located with a secondary molecule or reagent, such as an antibody, followed by imaging or detecting the secondary molecule or reagent.

In some embodiments, one or more of the cleavage products is conjugated to a cargo moiety that facilitates detection. In some embodiments, the cargo moiety is a fluorescent dye. In such embodiments, the cargo moiety may be detected directly using fluorescence microscopy, wherein cells, tissues, or entire subjects are placed in the field of a fluorescence microscope and visualized directly.

In some embodiments, the cargo moiety is a radioisotope. In such embodiments, the cargo moiety may be detected using X-rays, fluoroscopy, angiography, positron emission tomography (PET), or single positron emission computed tomography (SPECT) wherein cells, tissues, or entire subjects are placed in the field of the imaging modality and visualized.

In some embodiments, cleavage products are detected using a secondary molecule or reagent, e.g., an antibody, which specifically binds to or interacts with the cleavage products, e.g., an antibody that specifically binds to amino acid sequences in the cleavage products. In such embodiments, cells or tissues containing a cleavage product are contacted with a secondary molecule or reagent, followed by imaging or detecting the secondary molecule or reagent.

In some embodiments, detection of and assessing for the presence of the cleavage products is performed at the organismal level. In some embodiments, the detection and assessing is performed within a target region. In some embodiments, the detection occurs at extended time points post-administration (e.g., post-injection, such as post-intravenous administration), such as from 0.5 to 24 hours post-injection. In some embodiments, the detection occurs at multiple time points post-administration, e.g., post-injection.

### Screening Methods

The promolecules of the present disclosure find use in screening methods, e.g., in vitro or in vivo screening of candidate agents for a desired activity. In some embodiments, the present disclosure relates to methods for screening cells in vitro, e.g., to assess activity of a granzyme expressed by the cells, or to screen for candidate agents that modulate granzyme activity in the cells. In other embodiments, the present disclosure relates to in vivo screening methods that can be used, e.g., to screen candidate agents for the desired granzyme activity in transgenic animal models of disease.

In some embodiments, the screening methods of the present disclosure involve contacting cells in vitro with promolecules having X² linkers designed to be cleaved by a granzyme. Where granzyme-secreting immune cells are activated, the promolecules are cleaved and the cleavage product containing the membrane-interacting peptide undergoes a conformational change, typically forming an alpha-helical structure that interacts with phospholipid bilayers in the vicinity of the cell, thereby labeling regions in the vicinity of granzyme-secreting immune cell activation. A detectable moiety conjugated to the cleavage product comprising the membrane-interacting peptide can then be detected, which facilitates screening for granzyme-secreting immune cell activation. The amount of cleavage product that accumulates at a given location or position can therefore be used to screen for desired granzyme activity.

The methods of the present disclosure also relate to screening methods that can be used to identify candidate agents or test compounds having a desired activity, e.g., candidate agents that modulate granzyme activity or the activation of granzyme-secreting immune cells. In some embodiments, a screening method involves culturing cells in vitro and contacting the cells with a candidate agent or test compound. The cultured cells are then contacted with promolecules of the present disclosure comprising a cleavable linker that is cleaved by a granzyme of interest. Candidate agents or test compounds that elicit the desired granzyme activity in the cultured cells facilitate the production of cleavage-promoting conditions that result in cleavage of X². After cleavage of X², the cleavage product comprising the membrane-interacting peptide undergoes a conformational change to form an alpha-helical structure that inserts into and labels the nearby phospholipid bilayers. An increase in the level of labeling in the presence of the candidate agent or test compound as compared to the level of labeling in the absence of the candidate agent or test compound indicates that the candidate agent or test compound has the desired activity.

The methods of the present disclosure also relate to methods of screening cells in vivo, e.g., to identify candidate agents or test compounds having a desired activity, e.g., candidate agents that modulate granzyme activity or the activation of granzyme-secreting immune cells. For example, the screening methods discussed above may be conducted in vivo in animal models, e.g., transgenic animal models of disease, to identify cells or tissues of interest that express (or fail to express) a particular granzyme of interest, or to identify cells or tissues that modulate granzyme expression or activity in response to the candidate agent or test compound.

### Kits

Also provided by the present disclosure are kits for using the promolecules disclosed herein and for practicing the methods, as described above. The kits may be provided for administration of promolecules to a subject in which a disease or condition are to be diagnosed. The kit can include one or more of the promolecules and/or cargo moieties as disclosed herein, which may be provided in a sterile container, and can be provided in formulation with a suitable pharmaceutically acceptable excipient for administration to a subject. The promolecules can be provided in a formulation that is ready to be used as it is or can be reconstituted to have the desired concentrations. Where the promolecules are provided to be reconstituted by a user, the kit may also provide buffers, pharmaceutically acceptable excipients, and the like, packaged separately from the subject promolecules.

In addition to the above-mentioned components, the kits can further include instructions for using the components of the kit to practice the methods of the present disclosure. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### Example 1—Design and Synthesis of a Granzyme-Cleavable Restricted Interaction Peptide (GRIP)

Multiplex substrate profiling using mass spectrometry (MSP-MS) against recombinant human GZMB was performed to identify an optimal cleavage sequence to install in a RIP targeting GZMB. The MSP-MS library contains 228-tetradecameric peptides; a physicochemically diverse population of rationally designed substrates with maximum sequence diversity (FIG. 1, panel B). Based on the observation that most proteases require two optimally positioned amino acids for substrate recognition and cleavage, physicochemical diversity was generated in the peptide library through incorporation of all neighbor (XY) and near-neighbor (X*Y, X**Y) amino acid pairings. Upon incubation of native GZMB at various timepoints, cleavages were identified by peptide sequencing via liquid chromatography tandem mass spectrometry (LCMS-MS). A statistical analysis that considers both cleaved and uncleaved positions in the peptide library was subsequently performed to construct an iceLogo representation of the preferred substrate sequence spanning the granzyme B P4-P4′ sites (FIG. 1, panel C).

The iceLogo results suggested that four sequences with conserved sites of P2=P, P1=D, and P2′=S (i.e. XXPDXSXX) were equally specific and efficient GZMB substrates. The sequence IEPDVSQV (SEQ ID NO:57) was nominated for two reasons. First, the P4-P1 sequence was previously discovered to be specific to GZMB using an orthogonal approach, namely a positional scanning synthetic combinatorial library, and this sequence was shown to be specifically recognized by GZMB versus other human granzymes. Second, the IEPD tetrapeptide has been studied in vivo as part of a covalent reversible aldehyde radiotracer targeting GZMB, and the tetrapeptide-aldehyde appeared to be effective at labeling GZMB and stable in vivo.

The kinetics of IEPDVSQV (SEQ ID NO:57) cleavage by GZMB was assayed in vitro using a fluorescent quenched peptide substrate, and incorporating the P1′-P4′ sequence VSVQ significantly improved the kcat/Km compared to previously reported values for IEPD alone (˜8000 M⁻¹ sec⁻¹ versus ˜3300 M⁻¹ sec⁻¹, see FIG. 1, panel D). To generate the full length granzyme B-cleavable restricted interaction peptide (GRIP B) probe, this sequence was flanked using Temporin L (FVQWFSKFLGK; SEQ ID NO:3) as the membrane interacting domain, and the PAR1 (QDPNDQYEPF; SEQ ID NO:7) peptide as the masking domain (FIG. 1, panel E). Importantly, full length GRIP B was efficiently cleaved by recombinant human GZMB showing that neither Temporin L nor the masking domain interfered with proteolysis.

The substrate sequence FVQWFSKFLGK (SEQ ID NO:3) was assessed for specificity for granzyme B compared to thrombin, caspase 3, caspase 8, granzyme K, MMP9, and C1S at 5 nM concentrations with substrate at 60 μM, 37° C., and buffer of PBS, 1 mM DTT, and pH 7.4. As shown in FIG. 1, panel F, the substrate sequence is highly specific for granzyme B compared to the other proteases tested.

### Example 2—In Vitro Mechanisms and Radiosynthesis of ⁶⁴Cu-GRIP B

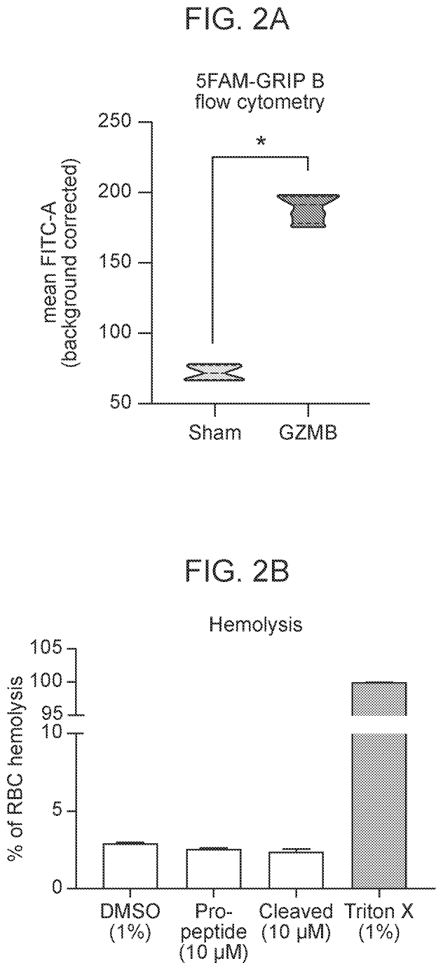

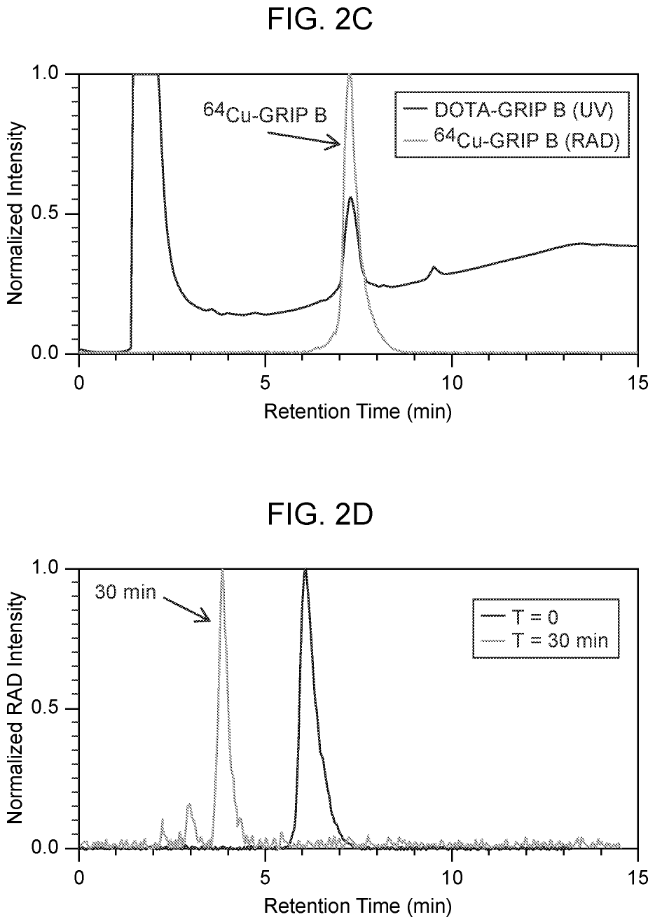

The proteolytically cleaved version of GRIP B was confirmed to effectively bind membranes. A N-terminal, 5FAM-tagged version of GRIP B was synthesized, and incubated with cells and recombinant human granzyme B or vehicle. Flow cytometry showed that intact GRIP B had low interaction with cell membranes, while co-incubating GRIP B with cells and 20 nM recombinant GZMB resulted in fluorescently labeled cell membranes (FIG. 2, panel A). The insertion of the cleaved GRIP B peptide into lipid micelles was further confirmed by measuring tryptophan fluorescence. Lastly, full length or proteolytically cleaved GRIP B was shown not to display toxicity toward human red blood cells in vitro (FIG. 2, panel B).

To couple GRIP B to a chelator for radiolabeling, the peptide was reacted with 1,4,7,10-tetraazacyclododecane-1,4,7,10-tetratacetic acid mono-N-hydroxysuccinimide ester (DOTA-NHS-ester) on solid support, which ligated to the amino group on the N-terminal phenylalanine. DOTA-GRIP B was subsequently deprotected, cleaved from resin, and purified with semi-prep HPLC.

DOTA-GRIP B was next radiolabeled with copper-64, since its half-life (t_{1/2}˜13 hours) would enable studies over a long window of time post injection to identify the optimal time point for imaging. ⁶⁴CuCl₂ was incubated with DOTA-GRIP B in HEPES buffer for 30 min at room temperature. The reaction was monitored for completeness via instant thin layer chromatography and purified using HPLC (FIG. 2, panel C). The decay corrected yield was consistently >95%, with a purity of >99%. The specific activity was ˜0.4 Ci/μmol over three radiosyntheses. Incubating ⁶⁴Cu-GRIP B with recombinant human GZMB showed conversion within 30 minutes to one radiolabeled product that comigrated on HPLC with the cold cleaved DOTA-peptide fragment (FIG. 2, panel D). Lastly, the serum stability was tested in vitro in mouse serum. On iTLC, ⁶⁴Cu-GRIP B was observed to be >98% stable over 4 hours at 37° C.

### Example 3—Immunomodulatory Therapies Induce Systemwide Changes in ⁶⁴Cu-GRIP B Biodistribution in Mouse Tumor Models

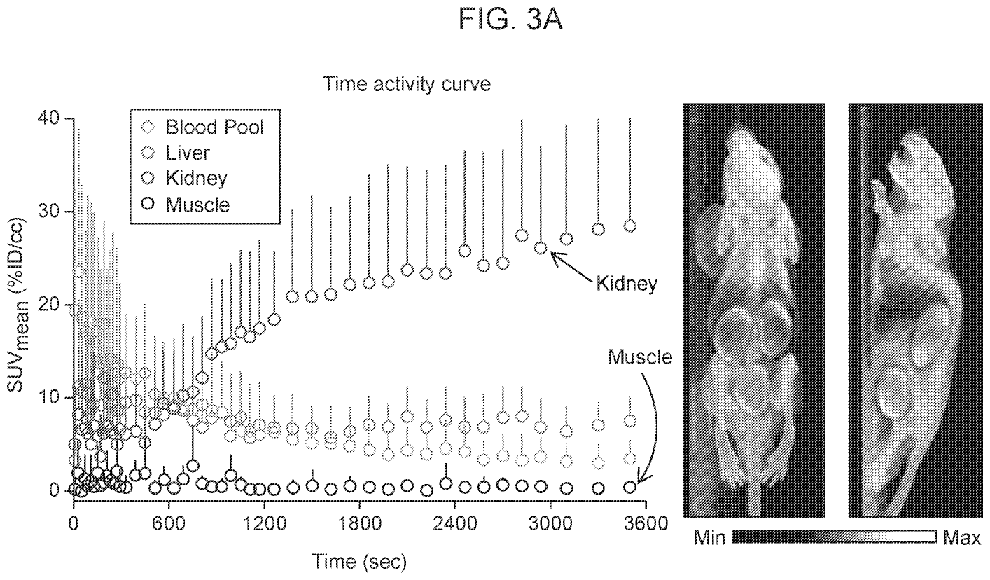

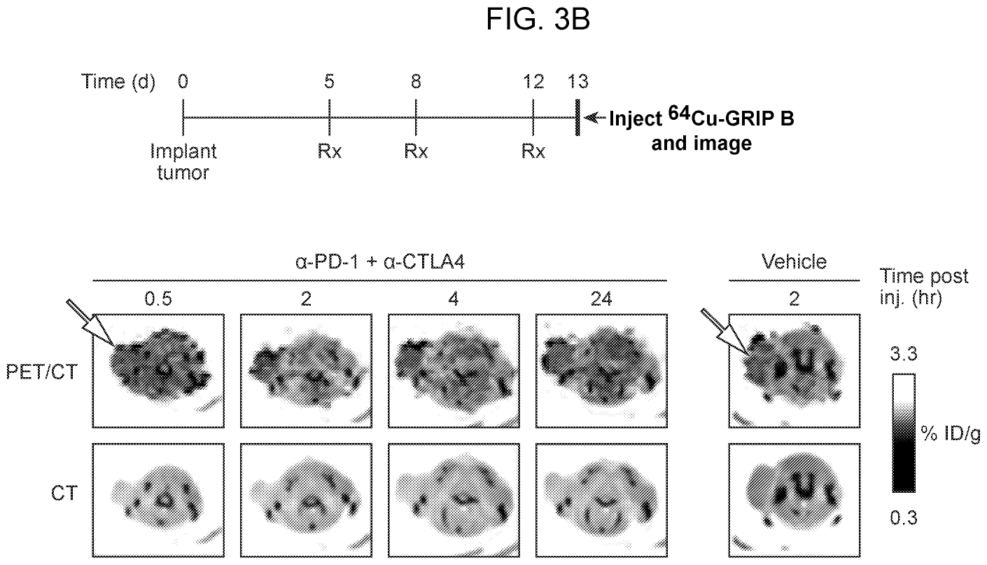

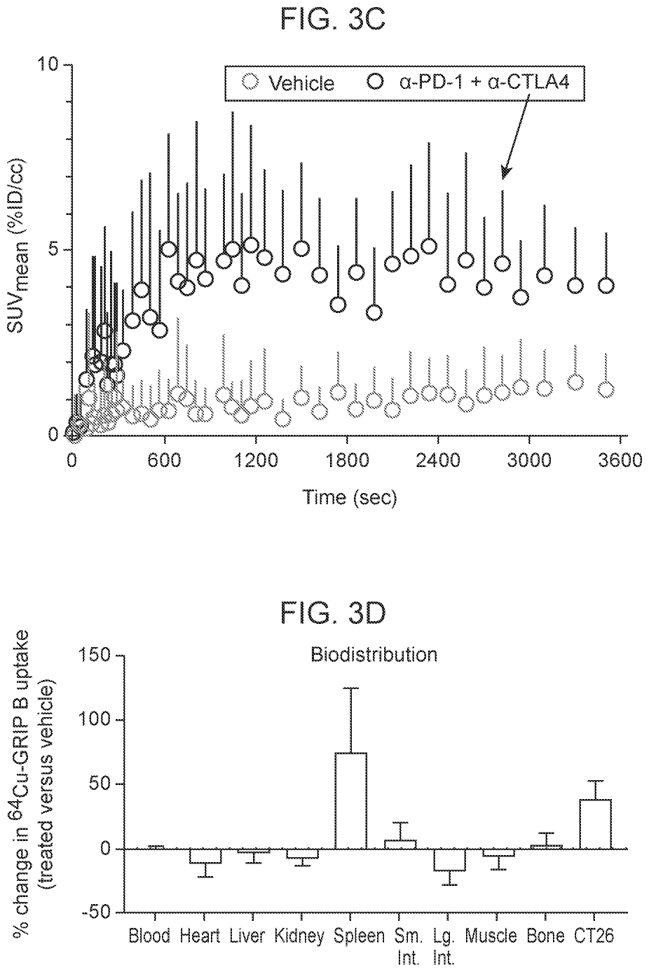

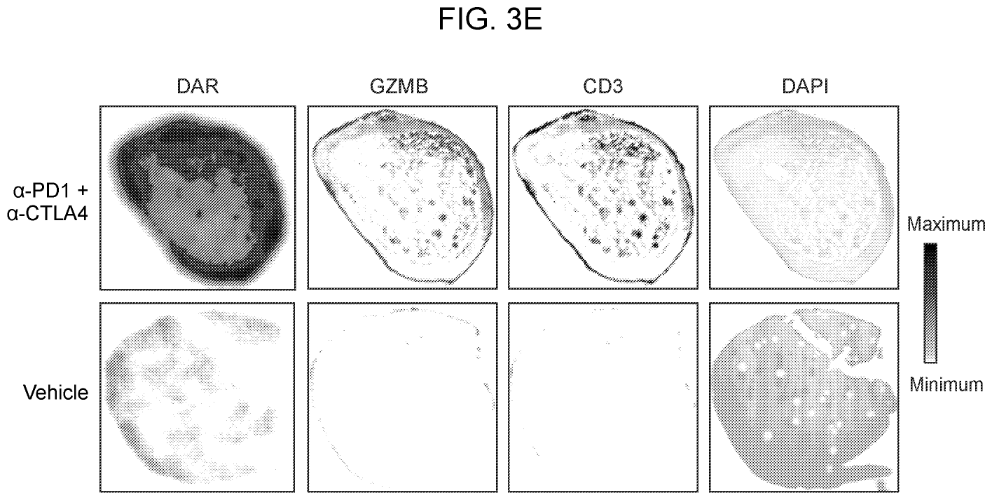

To understand tracer pharmacokinetics and normal tissue biodistribution, ⁶⁴Cu-GRIP B was first injected intravenously in C57B16/J mice followed by a 60 minute dynamic PET acquisition (FIG. 3, panel A). Region of interest analysis showed the probe cleared from blood pool with a t_{1/2}˜8 min. The dominant mode of clearance was renal, and the only substantial radiotracer accumulation outside of the kidneys was observed in liver. A biodistribution study was performed to evaluate radiotracer distribution in normal tissues out to 24 hours post injection. The biodistribution data corroborated the imaging findings showing the highest level of tissue-associated activity in the kidney and liver.

The effect of immunomodulatory therapies on ⁶⁴Cu-GRIP B biodistribution was next evaluated in mice bearing subcutaneous CT26 tumors, a mouse colorectal cancer cell line that responds to immunomodulatory therapy. Mice were treated with three intraperitoneal infusions of vehicle or anti-PD1 plus anti-CTLA4 CPIs over 11 days. The radiotracer was injected on day 14, and tumor uptake was monitored over several time points out to 24 hours post injection on PET. ROI analysis of static PET/CT images showed that ⁶⁴Cu-GRIP B uptake in the treated tumors steadily rose from 0.5 to 2-4 hours post injection (FIG. 3, panel B). Notably, radioactivity persisted in tumors out to 24 hours post injection, which is consistent with a mechanism of irreversible radiotracer trapping at the tumor. Moreover, tumoral uptake of ⁶⁴Cu-GRIP B was significantly higher in the CPI versus vehicle treated arm at 2 hours post injection. Time activity curves derived from a dynamic PET acquisition showed that tumoral accumulation of ⁶⁴Cu-GRIP B in CPI treated mice was rapid, reaching a level of ˜5% ID/cc within 10 min post injection (FIG. 3, panel C). Furthermore, compartmental modeling showed that k₃>>k₄, and k₄˜ 0, suggesting that ⁶⁴Cu-GRIP B is cleaved and trapped in tumors, as expected. By comparison, radiotracer uptake in vehicle tumors was significantly lower and did not change over time.

A biodistribution study at 2 hours post injection was performed to determine relative changes in tracer uptake between tissues in the vehicle and treated groups. These data showed ˜50% induction of radiotracer uptake in the tumors from treated mice compared to control mice (FIG. 3, panel D). A significant increase in tracer uptake within the spleen was observed, which is consistent with stimulation of T cells by systemic immune checkpoint inhibitors that we and others have documented. Digital autoradiography (DAR) showed that ⁶⁴Cu-GRIP B was significantly higher in the treated versus control tumor, and that the regions of radiotracer binding co-aligned with expression of GZMB and the T cell marker CD3 (FIG. 3, panel E).

### Example 4—Post Treatment Changes in ⁶⁴Cu-GRIP B are Attributed to GZMB Proteolytic Activity

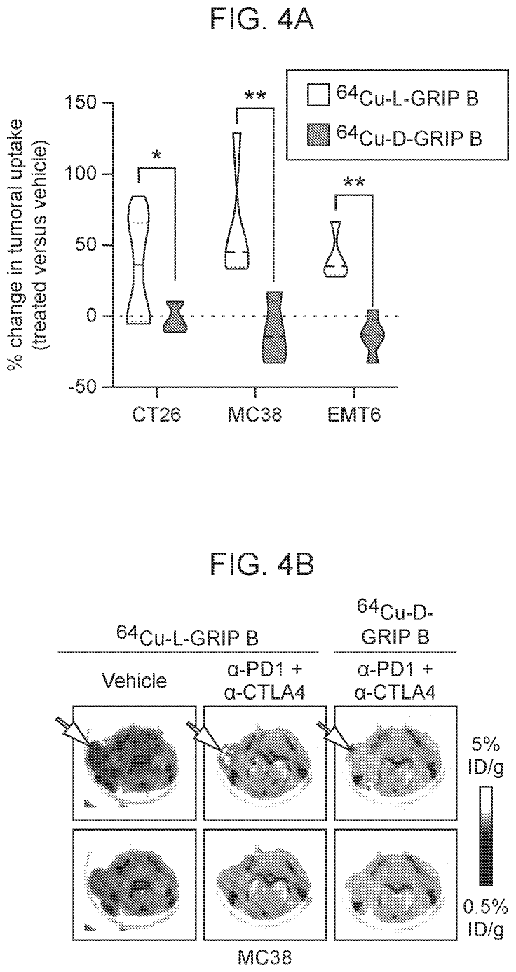

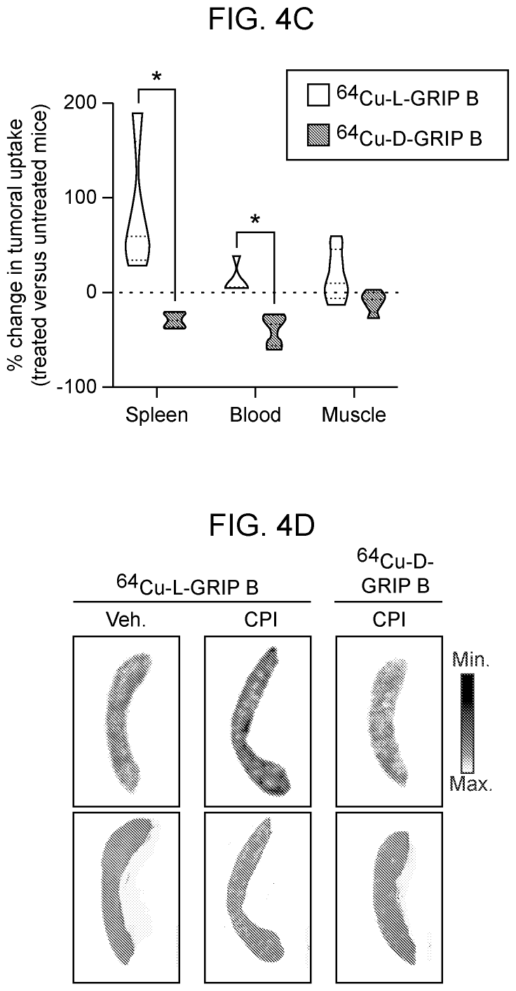

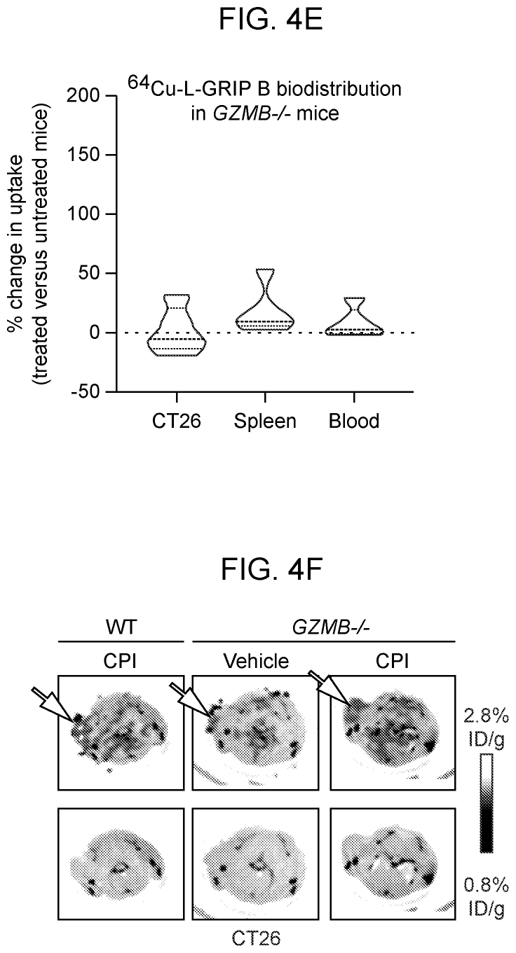

⁶⁴Cu-D-GRIP B, a probe that harbors a D-aspartic acid within the GZMB protease site (IEPdVSQV; SEQ ID NO:61) and prevents proteolysis by GZMB, was prepared to test if the post treatment “flare effect” requires proteolysis of ⁶⁴Cu-GRIP B. The probe was functionalized with DOTA and radiolabeled with Cu-64 using an approach similar to the synthesis of ⁶⁴Cu-GRIP B. Biodistribution studies showed that CPI treatment did not cause an increase in tumoral uptake of ⁶⁴Cu-D-GRIP B compared to control (FIG. 4, panel A).

Next, the biodistribution of ⁶⁴Cu-GRIP B and ⁶⁴Cu-D-GRIP B was compared in mice bearing MC38 (mouse colorectal cancer) or EMT6 (mouse mammary breast carcinoma) xenografts. The mice were treated with vehicle or anti-PD1 and anti-CTLA4 CPIs following the schema used for the CT26 cohort. Biodistribution data showed a significant increase in tumoral uptake of ⁶⁴Cu-L-GRIP B in both cohorts, while ⁶⁴Cu-D-GRIP B was not induced in tumors compared to controls, as expected (FIG. 4, panel A). Moreover, the absolute levels of ⁶⁴Cu-D-GRIP B uptake in treated tumors were low and comparable with baseline uptake of ⁶⁴Cu-L-GRIP B in untreated tumors (FIG. 4, panel B). Basal ⁶⁴Cu-D-GRIP B uptake in the spleen was also low and unaffected by treatment with immune checkpoint inhibitors (FIG. 4, panels C and D).

Lastly, to confirm that GZMB is responsible for post treatment changes in ⁶⁴Cu-GRIP B biodistribution, germline homozygous GZMB knockout mice were inoculated with CT26 tumors and the relative biodistribution of ⁶⁴Cu-L-GRIP B in mice was assessed after treatment with vehicle or CPIs. No significant post treatment changes in radiotracer uptake were observed among tumors and spleen exposed CPI compared to vehicle (FIG. 4, panels E and F).

### Example 5—Post Treatment Changes in Tumoral Uptake of ⁶⁴Cu-GRIP B Correlate with Tumor Volume Changes

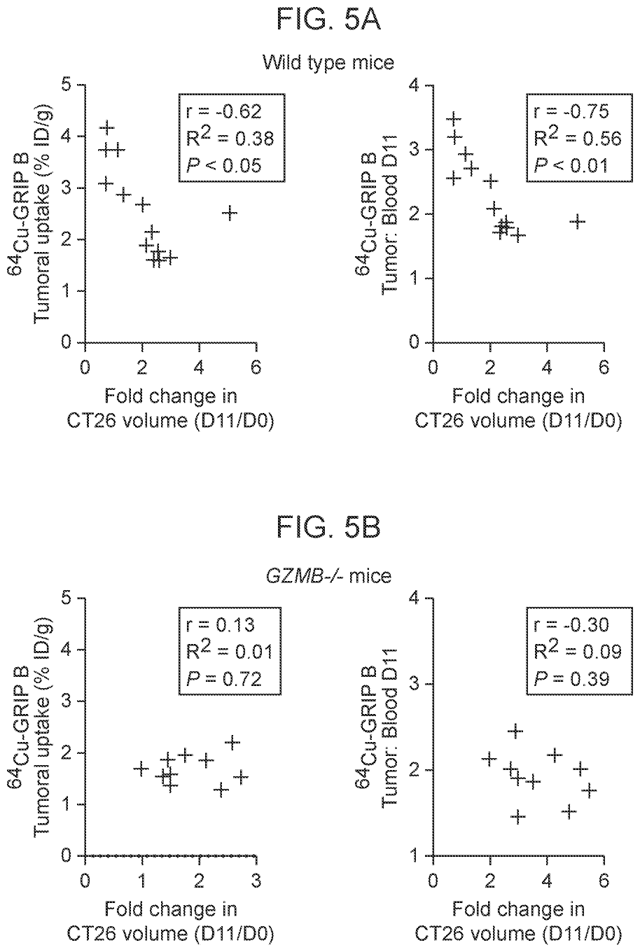

Tumors enriched with comparatively higher levels of GZMB activity could be expected to more significantly debulk compared to GZMB poor tumors. To confirm this, post treatment changes in tumoral uptake of ⁶⁴Cu-GRIP B were assessed for correlation with antitumor effects. The tumoral uptake of ⁶⁴Cu-GRIP B at day 11 was significantly correlated the percent change in tumor volume at day 11 compared to day 0 (FIG. 5, panel A). ⁶⁴Cu-GRIP B tumor to blood ratios of at day 11 also significantly correlated with percent change in tumor volume (FIG. 5, panel B). In contrast, neither tumoral uptake nor tumor to blood ratio of ⁶⁴Cu-GRIP B correlated with percent changes in tumor volume in the GZMB knockout mouse background (FIG. 5, panels C and D).

### Example 6—Studies with ⁶⁴Cu-GRIP B PET Suggest a Role for Secreted GZMB in Pulmonary Inflammation

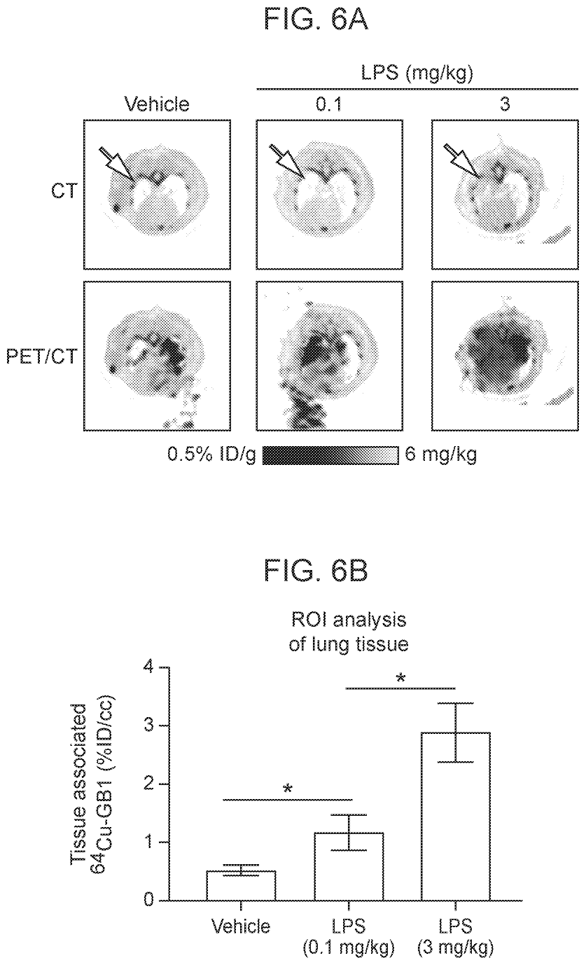

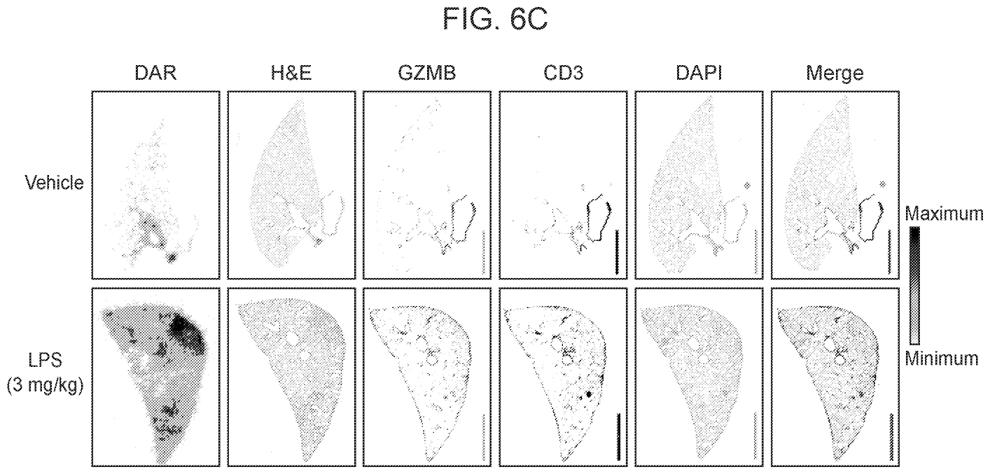

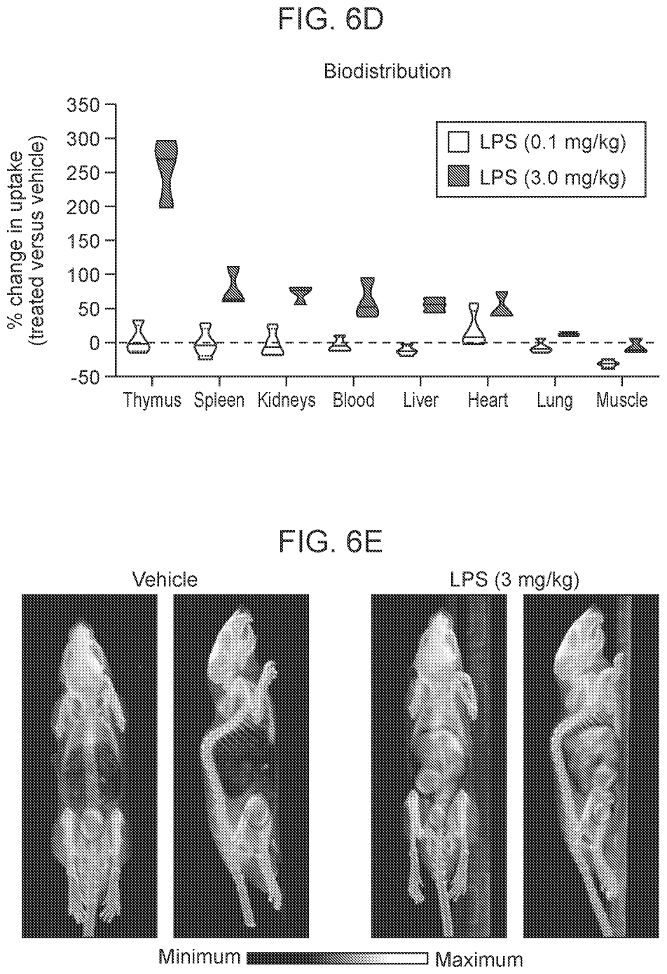

Though not well defined, non-cytotoxic functions for secreted GZMB have been proposed in several physiological processes, for example inflammation. To test if ⁶⁴Cu-GRIP B can localize potentially pathogenic reservoirs of secreted GZMB due to inflammation, ⁶⁴Cu-GRIP B PET/CT was performed on wild type mice that had received intratracheal instillations of lipopolysaccharide (LPS). PET/CT was performed four days after the instillation, a time point at which T cell recruitment to the lungs has occurred. ROI analysis showed significantly higher radiotracer accumulation in the lungs of mice treated with low (0.1 mg/kg) and high (3 mg/kg) doses of LPS compared to vehicle (FIG. 6, panels A and B). Autoradiography and immunofluorescence of the lungs showed visually higher radiotracer binding in the LPS treated lung, as expected, which also co-localized with GZMB and CD3 staining (FIG. 6, panel C). Since 3 mg/kg LPS can trigger systemwide T cell activation, radiotracer uptake was examined in a larger panel of mouse organs. Ex vivo biodistribution studies showed that ⁶⁴Cu-GRIP B was significantly higher in numerous tissues in the LPS treated versus vehicle mice at either dose, including lymphoid organs like the spleen and thymus (FIG. 6, panel D). Remarkably, the systemwide impact on T cell activation due to the intratracheal instillation of LPS was also visually obvious by comparing maximum intensity projections between treatment arms (FIG. 6, panel E).

### Example 7—Detection of Granzymes Secreted from Activated CAR T Cells by ⁶⁴Cu-GRIP B PET/CT

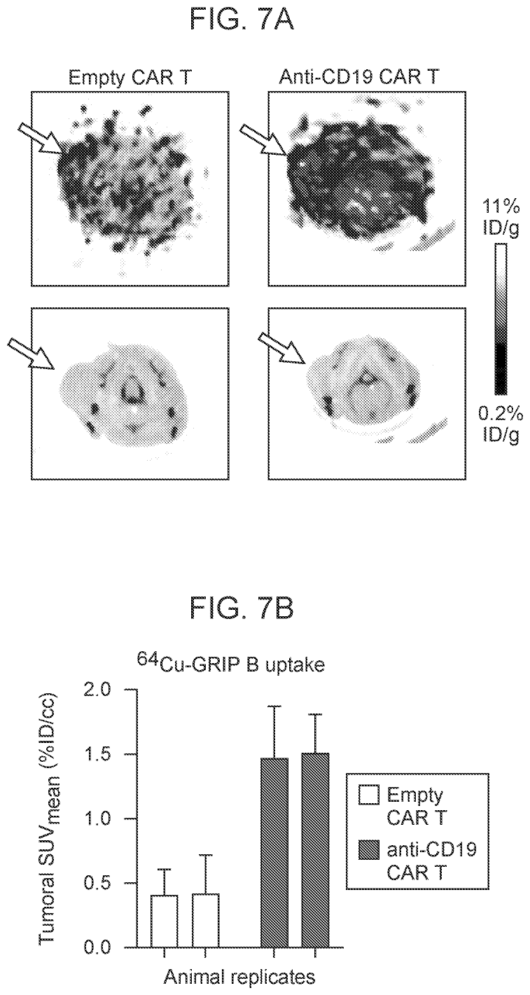

Mice bearing subcutaneous RAJI xenografts and administered anti-CD19 CAR T cells were imaged using ⁶⁴Cu-GRIP B PET/CT. Peripheral blood mononuclear cells were obtained from normal blood donor leukoreduction filters (Vitalant Blood Services). CD4+ and CD8+ T-cells were isolated via magnetic bead selection, activated with anti-CD3/CD28 beads, lentivirally transduced with a validated anti-CD19 CAR construct, and expanded in vitro with IL-2. In parallel, mice were injected with 1e6 Raji cells subcutaneously in the right flank. At tumor size of ˜400 mm3 5e6 CAR-expressing T-cells were implanted IV into each mouse. Mice received anti-CD19 CAR T cells or empty CAR Ts. After 6 days, the mice received ⁶⁴Cu-GRIP B (˜300 uCi/mouse) and were imaged from 0-4 hours post injection. The data depicted in FIG. 7 show images and region of interest analysis gathered at 4 hours post injection.

Additionally, mice bearing orthotopic RAJI xenografts and treated with activated CAR T cell based therapy were imaged using ⁶⁴Cu-GRIP B PET/CT. Raji cells (1e5) were administered orthotopically via tail vein injection to encourage seeding in abdominal tissues. This model was evaluated as subcutaneous Raji tumors are known to be intrinsically immunosuppressive.

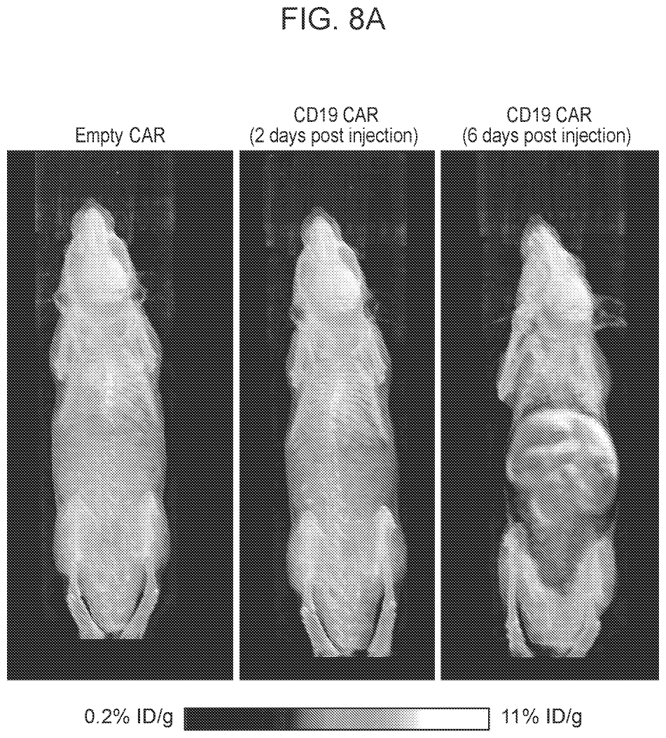

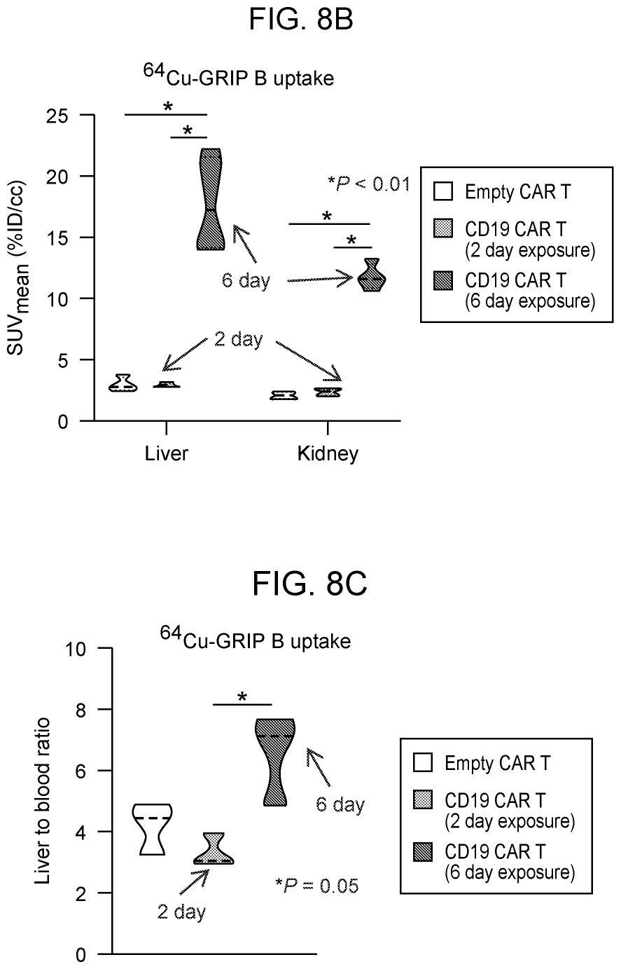

Peripheral blood mononuclear cells were obtained from normal blood donor leukoreduction filters (Vitalant Blood Services). CD4+ and CD8+ T-cells were isolated via magnetic bead selection, activated with anti-CD3/CD28 beads, lentivirally transduced with a validated anti-CD19 CAR construct (Wiita Ian, UCSF), and expanded in vitro with IL-2. One week after engrafting Raji cells subcutaneously, 5e6 CAR-expressing T-cells were implanted IV into each mouse. Mice received anti-CD19 CAR T cells or empty CAR Ts. After 2 or 6 days, the mice received ⁶⁴Cu-GRIP B (˜300 uCi/mouse) and were imaged from 0-4 hours post injection. The data depicted in FIG. 8 show images and region of interest analysis gathered at 4 hours post injection. FIG. 8, panel C shows post mortem liver dosimetry. Collectively, these data show that ⁶⁴Cu-GRIP B can detect granzyme B production from activated CAR T cells.

### Example 8—Detection of a Productive Immune Response in a Pneumonia Model by ⁶⁴Cu-GRIP B PET

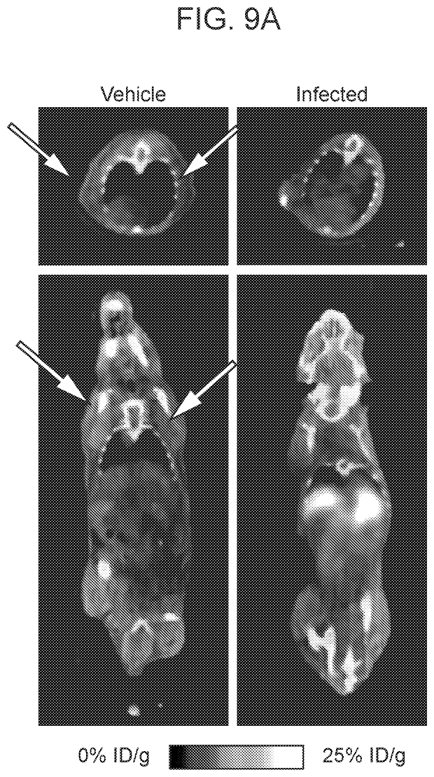

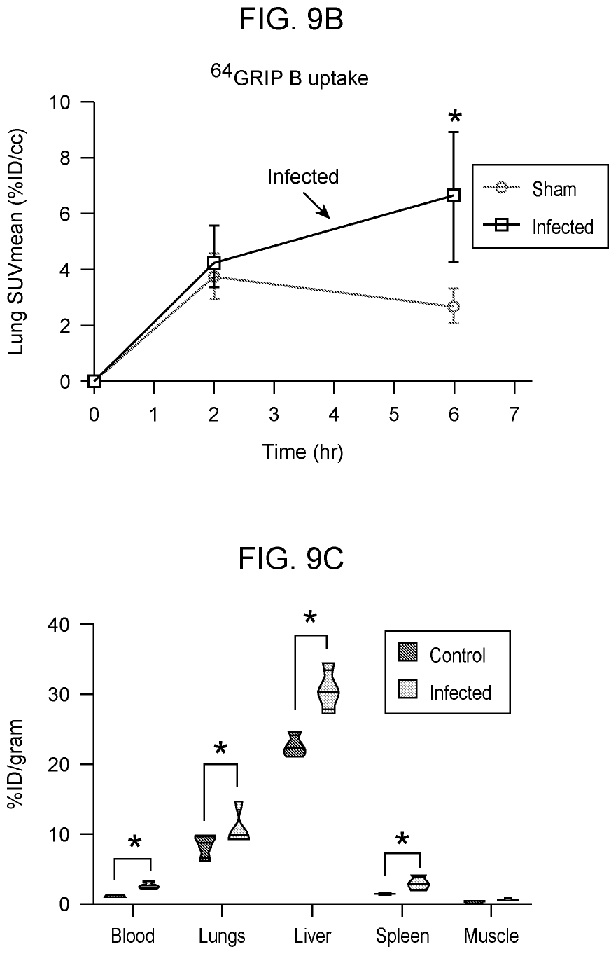

It was next evaluated whether ⁶⁴Cu-GRIP B PET can detect a productive immune response in a pneumonia model. In collaboration with the Looney lab at UCSF, mice received an intranasal instillation of virus or sham, and were imaged with ⁶⁴Cu-GRIP B at 10 days post infection (the time point at which peak recruitment of T cells to the lungs occurs). As shown in FIG. 9, the radiotracer uptake in infected lungs is very high and significantly different than healthy lungs at 6 hours post radiotracer injection. Moreover, high tracer uptake was noted on PET in other organs, for example the liver (*P<0.01). This unexpected finding led to a biodistribution study in lungs and other organs. Comparing relative radiotracer uptake per organ showed that viral infections induce higher radiotracer uptake in numerous tissues, including the spleen, liver, and blood pool (*P<0.01). Currently, granzyme B expression in these tissues is being confirmed using autoradiography and IF, and control studies (D-amino acid probe, imaging in germline granzyme B knockout mice) are being performed to confirm the pathogen induced changes in tracer biodistribution are driven by granzyme B.

### Example 9—Detection of Granzyme B Secreted from Activated Immune Cells in Response to Bacterial Infections by ⁶⁴Cu-GRIP B PET

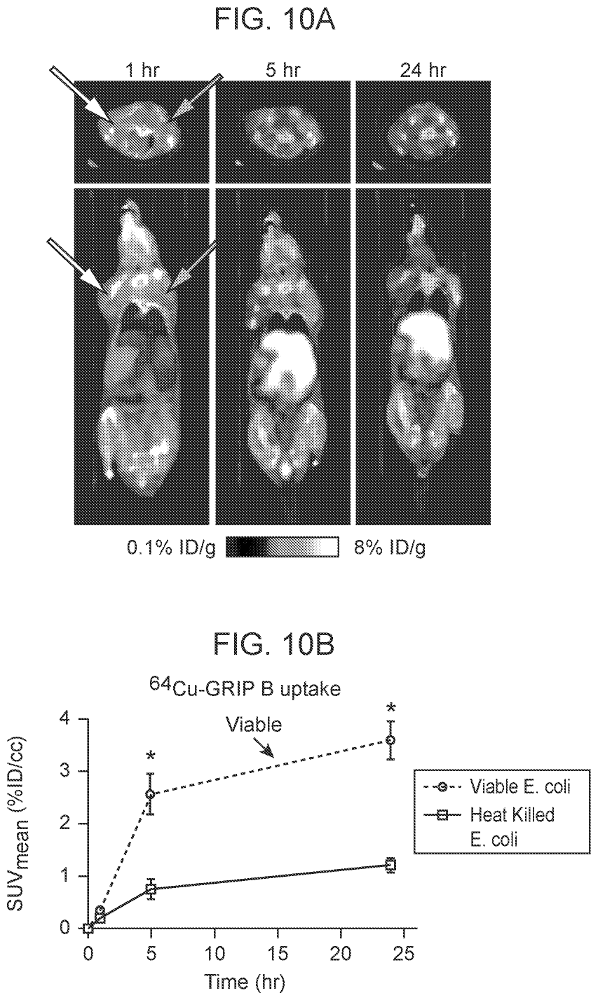

⁶⁴Cu-GRIP B was also tested to determine whether it can detect granzyme B secreted from activated immune cells attempting to combat bacterial infections. A cohort of mice bearing bilateral deltoid implants of live E. coli or heat killed E. coli was established to test the kinetics of tracer uptake (FIG. 10). Rapid tracer uptake was observed in the myositis lesion, which increased out to 5 hours and persisted to at least 24 hours post injection. Moreover, the ⁶⁴Cu-GRIP B uptake was significantly higher in the live E. coli abscess compared to the site of heat killed bacterial implantation.

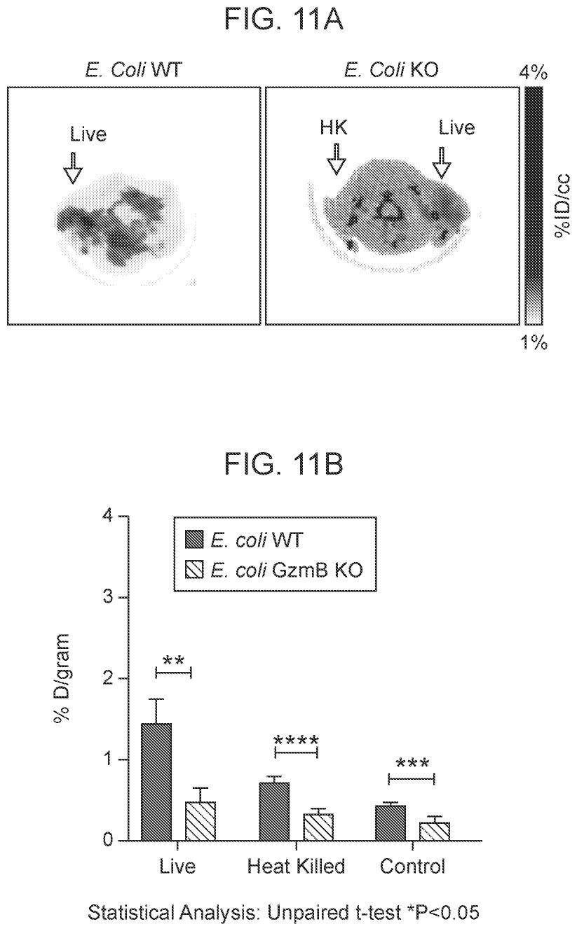

To confirm the radiotracer uptake in the abscess was due to granzyme B, imaging studies were performed in germline GZMB knockout mice. The mice received live and heat killed E. Coli and were injected and imaged following the same protocol for wild type mice. PET/CT and biodistribution data showed that ⁶⁴Cu-GRIP B uptake was lower in the infected muscle of GZMB knockout mice compared to wild type (FIG. 11). Moreover, there was negligible difference between tracer uptake in normal muscle, muscle treated with live E. Coli, or mice treated with heat killed E. Coli in the knockout strain.

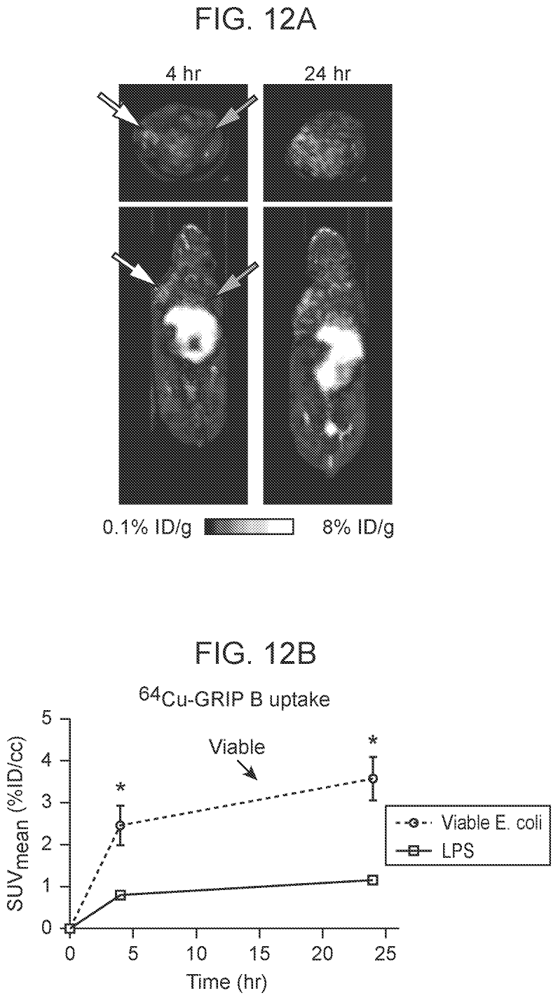

Next, the granzyme B responses to live E. coli versus treatment with a bolus of the endotoxin LPS were compared. Three to four hours after implantation, mice received ⁶⁴Cu-GRIP B and were imaged serially (FIG. 12). Remarkably, the live E. coli abscess was significantly more immunostimulatory than LPS, and differences in radiotracer uptake were detected from 6-24 hours post injection.

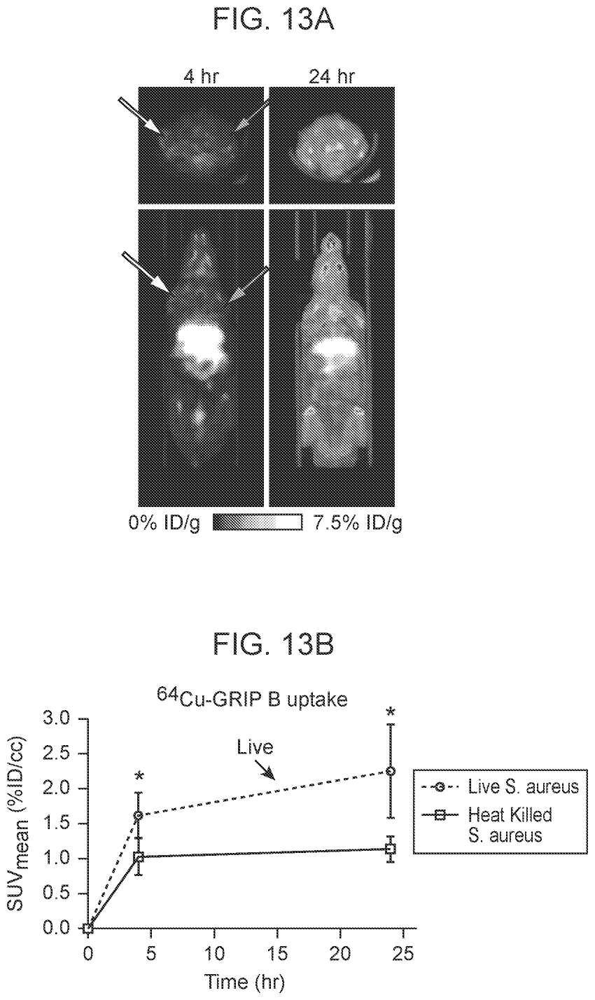

Granzyme B responses to other bacterial strains has also been tested. Live or heat killed S. aureus were implanted into the deltoids of mice to understand immune responses on PET. It was found that ⁶⁴Cu-GRIP B accumulation parallels what has been observed in E coli infections, with radiotracer uptake rising rapidly from 0-6 hours post injection and plateauing from 6-24 hours (FIG. 13). Moreover, radiotracer uptake in the live bacterial abscess was significantly higher than the heat killed abscess, as expected based on the E coli data. Interestingly, a more modest granzyme B response was detected in the live bacterial abscess compared to that of E. coli. One possibility for the difference in response is that granzyme B is not uniformly employed to attack all pathogens, and the immune cells may customize their granzyme response based on the features of the pathogen.

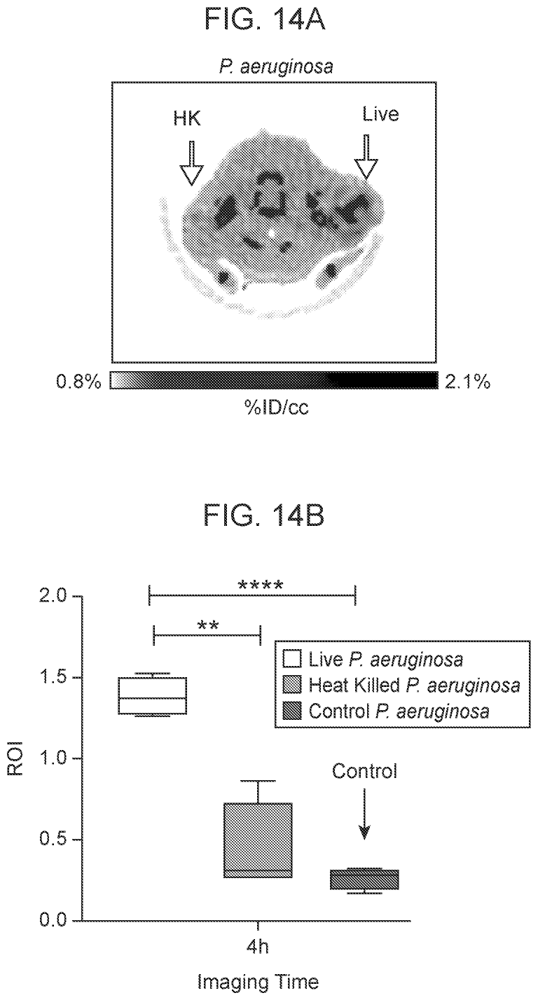

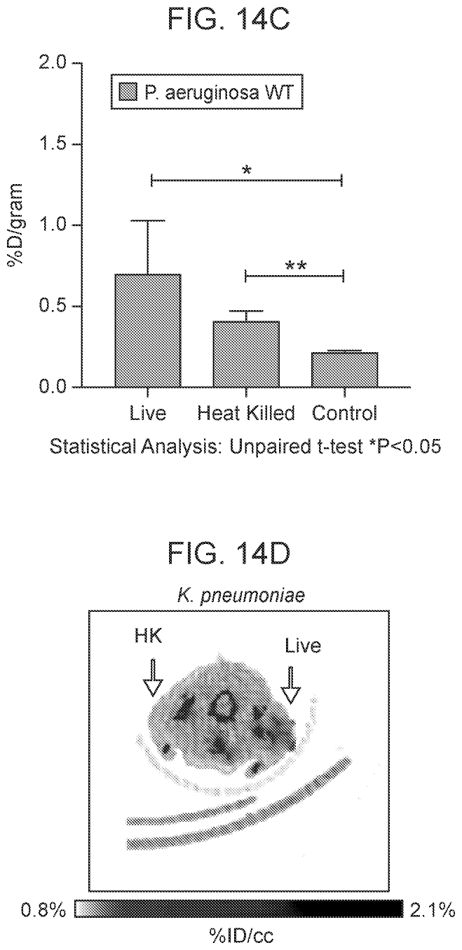

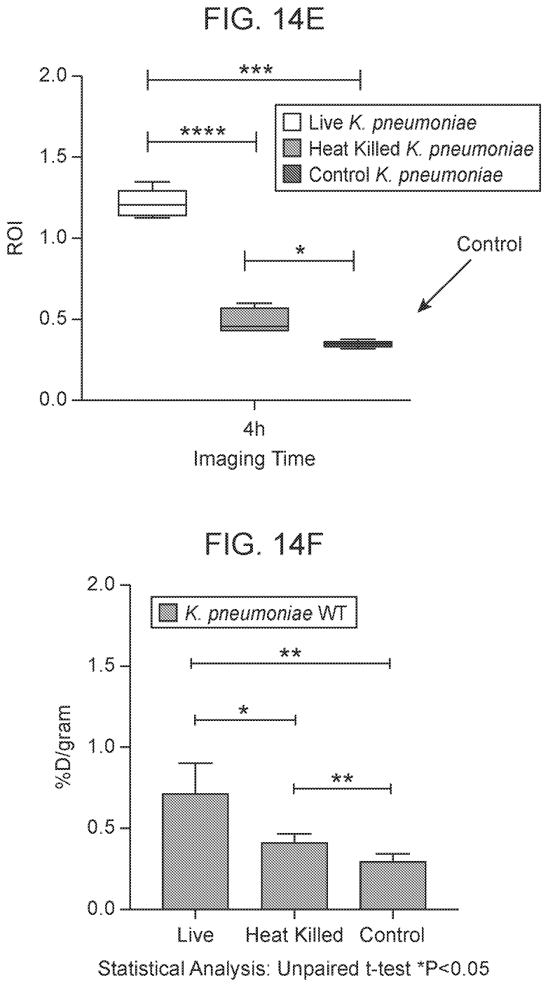

Performing additional myositis studies with other bacterial strains yielded unexpected and variable granzyme “responses” on PET. For example, both P. aeruginosa and K. pneumoniae displayed qualitatively similar findings to E. coli and S. aureus infection (FIG. 14). ⁶⁴Cu-GRIP B uptake in live bacterial infections in the deltoid muscles were higher than was observed in the contralateral deltoid muscle which was exposed to heat killed bacteria. In both mouse cohorts, radiotracer uptake in the treated muscles was higher than in the normal muscle.

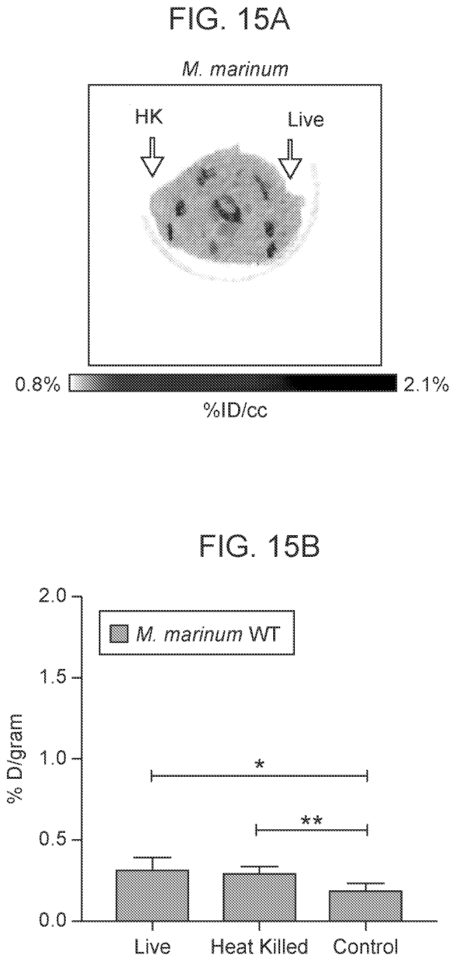

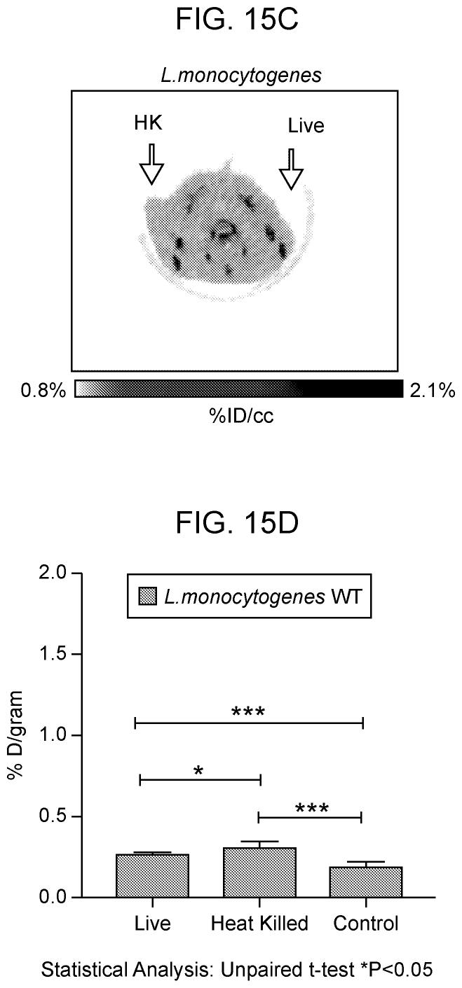

Two bacterial species did not impact ⁶⁴Cu-GRIP B biodistribution in vivo (FIG. 15). Neither M. marinum nor the L. monoctyogenes induced ⁶⁴Cu-GRIP B uptake in live bacterial abscesses compared to heat killed controls. A significant increase in radiotracer uptake was observed when compared to normal muscle, which likely reflects an inflammatory response to the foreign agents.

### Methods

### General Methods

All reagents were purchased from commercial sources and used without further purification. ⁶⁴Cu-hydrochloride acid was purchased from University of Wisconsin Madison. Recombinant human GZMB was purchased from Sigma Aldrich. The mouse cancer cell lines CT26 and EMT6 were purchase from ATCC. MC38 was purchased from Kerafast. Anti-mouse PD-1 (CD279) (BE0146) and anti-mouse CTLA-4 (CD152) (BE0164) were purchased from Bio X Cell; Anti granzym B (ab4059) was purchased from Abcam; anti-CD3 (MCA1477) was purchased from Bio-Rad; AF488 anti-Rabbit (A21206), AF546 anti-Mouse (A1 11081) and AF633 anti-Mouse (A21052) secondary antibodies were purchased from Invitrogen. DAPI (D1306) was purchased from Life Technologies Corporation. Antibodies for immunofluorescence. All cell lines were cultured according to manufacturer's instructions.

### Multiplex Substrate Profiling by Mass Spectrometry

Human GZMB (100 nM) was incubated with a library containing 228 synthetic tetradecapeptides (500 nM). Aliquots (10 μL) were removed at three time intervals and subsequently quenched with 10 μL of 8M guanidinium hydrochloride. Aliquots were then flash frozen until all timepoints were taken. Prior to mass spectrometry, samples were desalted using C18 tips (Rainin). Aliquots were then analyzed by LC-MS/MS sequencing using a Quadrupole Orbitrap mass spectrometer (LTQ Orbitrap XL) coupled to a 10,000 psi nanoACQUITY Ultra Performance Liquid Chromatography (UPLC) System (Waters) for peptide separation by reverse phase liquid chromatography (RPLC). Peptides were separated over a Thermo ES901 C18 column (75-μm inner diameter, 50-cm length) coupled to an EASY-Spray™ ion source and eluted by applying a flow rate of 300 nL/min with a 65-minute linear gradient from 2-50% in Buffer B (acetonitrile, 0.5% formic acid). Survey scans were recorded over a 325-1500 m/z range and up to the three most intense precursor ions (MS1 features of charge 2) were selected for higher energy collisional dissociation (HCD) at a resolution of 30,000 at m/z 200 for MS/MS[CB2]. Data was acquired using Xcalibur software and processed as previously described. Briefly, raw mass spectrometry data was processed to generate peak lists using MSConvert. Peak lists were then searched in Protein Prospectorv.6.2.2 against a proprietary database containing the sequences from the 228 tetradecapeptide library. Searches used a mass accuracy tolerance of 20 ppm for precursor ions and 30 ppm for fragment ions. Variable modifications included N-terminal pyroglutamate conversion from glutamine or glutamate and oxidation of tryptophan, proline, and tyrosine. Searches were subsequently processed using the MSP-xtractor software (http://www.craiklab.ucsf.edu/extractor.html), which extracts the peptide cleavage site and spectral counts of the corresponding cleavage products. Spectral counts were used for the relative quantification of peptide cleavage products. Human GZMB samples were processed as three biological replicates per time point and a non-enzyme control was used for each replicate to remove unspecific cleavages from data analysis.

### Fmoc-Solid-Phase Peptide Synthesis

A quenched fluorogenic peptide synthesized of the sequence NH2-K(MCA)IEPDVSQVK(DNP)-COOH (SEQ ID NO:62) was synthesized by Fmoc solid phase synthesis on a Biotage Syroll peptide synthesizer at ambient temperature. The synthesis scale was at 12.5 μM using preloaded lysine(2-dinitrophenyl) Wang resin where the DNP quencher was linked to the epsilon nitrogen of the lysine. Coupling reactions were carried out with 4.9 equivalents of HCTU (O-(1H-6-chlorobenzotriazole-1-yl)-1,1,3,3-tetramethyluronium hexafluorophosphate), 5 equivalents of Fmoc-amino acid-OH, and 20 equivalents of N-methylmorpholine (NMM) in 500 μL N,N-dimethylformamide (DMF) for 8 minutes while shaking. Each amino acid position was double coupled and subsequent Fmoc deprotection was carried out with 500 μL of 40% 4-methylpiperadine in DMF for 10-minutes followed by 6 washes with 500 μL DMF for 3-minutes. The final amino acid coupling contained the fluorophore, lysine (7-methoxycoumarin-4-acetic acid (MCA)) where MCA was linked to the epsilon nitrogen of the lysine. Peptides were cleaved from Wang's resin with 500 μL of solution composed of 95% trifluoroacetic acid, 2.5% water, and 2.5% triisopropylsilane for 1-hour while shaking. Crude peptide product was then precipitated in 30 mL cold 1:1 diethyl ether:hexanes and then solubilized in a 1:1:1 mixture of DMSO:water:acetonitrile. Solubilized crude was purified by high-performance liquid chromatography (HPLC) using an Agilent Pursuit 5 C18 column (5 mm bead size, 150×21.2 mm) on an Agilent PrepStar 218 series preparative HPLC. Mobile phase A and B were water+0.1% TFA and acetonitrile+0.1% TFA, respectively. Purified peptide product had solvent removed under reduced atmosphere and was solubilized into a DMSO stock with a final concentration of 10 mM. Purity was confirmed by liquid chromatography-mass spectrometry and the stock was stored at −20° C. Fluorescently labeled 5FAM-GRIP B was purchased from CPC Scientific at 95-98% purity.

### Synthesis of DO TA-GRIP B

DOTA-GRIP B (Dota-hexanoic acid-FVQWFSKFLGKIEPDVSQVQDPNDQYEPF-COOH; SEQ ID NO:63) was synthesized first using standard solid phase peptide synthesis conditions as outlined above. Resin-bound peptide with N-terminal hexanoic acid was triple coupled with two equivalents of dota-NHS, five equivalents of HCTU, and twenty equivalents of N,N-Diisopropylethylamine (DIPEA) for 12-hours. DOTA-GRIP B probe was then cleaved, purified, and analyzed as described for the fluorogenic peptide.

### In Vitro Kinetics

Kinetic measurements were carried out in Corning black 384 well flat bottom plates and read on a BioTek H4 multimode plate reader. Proteolysis of the quenched fluorogenic peptide (NH2-K(MCA)IEPDVSQVK(DNP)-COOH; SEQ ID NO:62) by GZMB was carried out at a final enzyme concentration of 40 nM in PBS. Kinetics were carried out at 37° C. and activity was monitors for 1 hr. Vo was calculated at 1 min and 30 min in RFU/s. Initial velocities were then converted to M/s using a standard curve of cleaved substrate.

### Intrinsic Tryptophan Fluorescence Spectroscopy Measuring Lipid Insertion

The fluorescence of the tryptophan within full length and activated GRIP B was monitored in the presence or absence of lipid micelles on a BioTek H4 multimode plate reader. Sodium dodecyl sulfate (SDS) was solubilized as a 5 mg mL⁻¹ stock. Full-length and activated GZMB-RIP were solubilized in PBS to a final concentration of 0.01 mg mL⁻¹ with a final peptide:lipid molar ratio of 1:40. Tryptophan emission spectra of the peptide:lipid suspension were acquired with an excitation wavelength of 295 nm and by scanning from 310 to 450 nm. The bandwidth was 5 nm for both excitation and emission. The spectrums of the peptides in PBS in the absence of SDS lipids were acquired at the same concentration of 0.01 mg mL⁻¹.

### Toxicity Assay Measuring Hemolysis of Human Erythrocytes

Blood from healthy anonymous donors was harvested from Trima Leukoreduction chambers (Vitalant, San Francisco CA). Erythrocytes were isolated from the anonymous blood samples. Full-length GRIP B and activated GRIP B were measured for their hemolytic activity on healthy human erythrocytes in triplicate. Aliquots of human erythrocytes were suspended in PBS (pH 7.4) and incubated with serial dilutions of both peptides which were initially solubilized in DMSO. DMSO and 1% Triton X-100 were incubated in parallel as negative and positive controls, respectively. Incubation was for 1 hr at 37° C. After incubation, the samples were centrifuged for 5 min at 2,000×g upon which the supernatant was collected. Supernatant was measured for the release of hemoglobin by the erythrocytes using a BioTek H4 multimode plate reader, monitoring the optical density of the supernatant at a wavelength of 540 nm.

Flow Cytometry with 5FAM-GRIP B

MC38 cells (2×10⁵/well) were seeded into 12-well plate and incubated at 37° C. for 48 hours. 5FAM-GRIP B (200 nM) and GZMB (20 nM) were dissolved in HBSS and incubated at 37° C. for 2 hours. 200 nM RIP with/without 50 nm GZMB in HBSS (300 mL) was added into the well with the cells, followed by incubation at 37° C. for 30 min. The probe solution was removed, and cells were washed with PBS 4-5 times. Trypsin (100 mL) was added, followed by 3 min incubation at 37° C. PBS was added into the wells, and all cells were collected and washed with PBS for one time before further diluting with PBS (300 mL) and passed through a cell strainer. Experiments were performed on a BD FACSCanto™ II Cell Analyzer. Data was analyzed by using FlowJo and Prism 8.0.

### Radiosynthesis and In Vitro Characterization of ⁶⁴Cu-GRIP B

Into a 1.5 mL reaction vial was added 5 mCi of ⁶⁴Cu-chloride (aq.) and the pH was adjusted to 7.0 with Na₂CO₃ (2 M). A solution of DOTA-GRIP B (50 μg in 20 μL DMSO) and 0.1 M NH₄OAC buffer (200 μL) was added into this reaction vial. The reaction mixture was incubated at 50° C. for 30 minutes. Reaction progress was monitored by analytical HPLC equipped with Agilent Pursuit analytical column (C18, 200 Å, 4.6 mm×10 cm, 5 μm) or Phenomenex Luna® analytical column (C18, 100 Å, 4.6 mm×250 cm, 10 μm) (70:30 MeOH:H₂O to 95:5 MeOH:H₂O over 10 min). The crude reaction was purified using a C18 Sep-Pak cartridge, and eluted with a small volume of CH₃CN. CH3CN was then removed at 50° C. under vacuum and a gentle stream of N₂(g) to afford neat ⁶⁴Cu-GRIP B. The chelation efficacy is usually >90% based on the HPLC. A formulation comprising 10% DMSO, 10% tween 80 and 80% saline was adopted for the further mice studies. Cleavage of ⁶⁴Cu-GRIP B by granzyme B was verified in vitro by adding the radiotracer (˜200 Ci) in to recombinant Granzyme B (10 nM) in 500 uL PBS. The vial was then incubated at 37° C. Rad-HPLC was used to monitor the cleavage of the radiotracer at the dedicated time points.

### Animal Studies

All animal experiments were approved by the Institutional Animal Care and Use Committee at UCSF. Four to six week old male or female balb/c mice and C57BL6/J mice were purchased from Jackson Laboratory and housed with free access to the water and food. All mice were inoculated with 5×10⁶ CT26, MC38, or EMT6 cells in a mixture of media and Matrigel (Corning) (v/v 1:1) subcutaneously into the left shoulder. Anti-mouse PD-1 (CD279) (BE0146) and anti-mouse CTLA-4 (CD152) (BE0164) were purchased from Bio X Cell and stored at 4° C. during the treatment studies. Mice bearing subcutaneous tumors were received anti-mouse CTLA-4 (200 ug) or/and anti-mouse PD-1 (200 ug) and as a combination therapy or PBS as the vehicle on days 5, 8, 11 following the tumor inoculation. Mice were weighed and the tumor volume were measured with calipers on the same day of the treatment. On day 14, all mice were used for PET/CT or BioD studies.

### Small Animal PET/CT

⁶⁴Cu-L-GRIP B or ⁶⁴Cu-D-GRIP B (˜100 μCi/mouse) in 100-150 μL of 10% DMSO and 10% Tween 80 in saline was injected via tail vein. After a period of uptake time, mice were anesthetized with isoflurane (˜2%), and imaged with a microPET/CT scanner (Inveon, Siemens). For static imaging, mice were scanned for 30 min for PET data acquisition and 10 min for CT data acquisition. For the dynamic acquisitions, the mice were anesthetized, positioned on the scanner bed, and injected intravenously with radiotracer. The dynamic acquisition was performed for scanned for 60 min followed by a 10 min CT acquisition.

List-mode PET data were histogrammed to generate sinograms that were reconstructed using a 2D ordered subsets expectation maximization algorithm provided by the scanner manufacturer. Attenuation correction was applied using the co-registered CT data that were acquired immediately following PET data acquisition. CT was acquired using the following setting: 220 degree angular coverage with 120 steps, x-ray tube operating at 80 kVp and 0.5 mA with each angular step exposure time set as 175 ms. All reconstructed 3D PET volume image voxels were calibrated to Bq/ml using a precalibrated quantification factor. AMIDE software was used for reconstruction of PET/CT data and image analysis.

### Biodistribution Studies

At dedicated time points post radiotracer injection, mice were euthanized with CO2(g) asphyxiation, and the blood was collected by direct cardiac puncture. Tissues were harvested, weighed and counted on a gamma counter (Hidex). The amount of radioactivity in the tissues was determined by comparison with a standard of known activity. The samples were decay-corrected and expressed as the percentage of the injected dose/weight of the harvested tissues (% ID/g).

### Digital Autoradiography

Tumors or designated tissue were flash frozen in OCT in dry ice. The tissues were sectioned with a microtome (Leica) into slices with 10-20 um thickness and directly mounted on glass slides (VWR). GE Storage Phosphor Screen were exposed by such slides with radioactive tissue. After 10 half-live of copper-64, the screen was developed on a phosphorimager (Typhoon 9400). The images were further analyzed by using Fiji software.

### Histology

H&E staining and IF staining were performed by the Pathology core facility at UCSF and Acepix Biosciences (Hayward, CA). For immunofluorescence studies, tumor samples were soaked in acetone −20° C. for 20 min, followed by in MeOH 4° C. for 10 min. Antigen retrieval was conducted with Citrate Buffer 10 mM pH=6 and samples were blocked with universal blocking buffer plus 5% goat and donkey serum. The primary antibodies: anti-GZMB (ab4059, Abcam) (1:50), anti-CD3 (MCA1477, Bio-Rad) (1:100) were added into samples and incubated at 4° C. overnight. Such primary antibodies were detected by AF488 anti-Rabbit (A21206, Invitrogen) (1:200); AF546 AF546 anti-Mouse (A1 11081, Invitrogen) (1:200) and AF633 anti-Mouse (A21052, Invitrogen) (1:200) secondary antibodies by incubating with samples. DAPI Nucleic Acid Stain (D1306, Life Technologies Corporation) was used to stain the nucleus by incubating with samples (10 min at room temperature. Immunofluorescence results were performed by the Gladstone Institutes' Histology & Light Microscopy Core. Images of whole sections were acquired on a VERSA automated slide scanner (Leica Biosystems, Wetzlar, Germany), equipped with an Andor Zyla 5.5 sCMOS camera (Andor Technologies, Belfast, UK). Individual images were created with the ImageScope software (Aperio Technologies, Vista, CA).

### Statistics

All statistical analysis was performed using PRISM v8.0 or ORIGIN software. Statistically significant difference was determined by an unpaired, two-tailed Student's T test. Changes only at the 95% confidence level (P<0.05) were regarded as statistically significant.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A promolecule comprising the structure, from N-terminal to C-terminal or C-terminal to N-terminal, 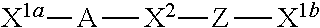
wherein:
X^{1a} and/or X^{1b} may be present or absent, and when present comprise a nucleophilic moiety;
A is a membrane-interacting polypeptide portion that, when separated from portion Z, comprises an alpha-helical structure capable of inserting into a phospholipid bilayer;
Z is a polypeptide that, when linked to portion A through portion X², is effective to inhibit interaction of portion A with a phospholipid bilayer; and
X² is a granzyme-cleavable linker, wherein X² joins portion A to portion Z, and wherein X² can be cleaved under physiological conditions.

2. The promolecule of claim 1, wherein Z is a polypeptide comprising the amino acid sequence SFLL(X^{Z})NPNDKYEPFW (SEQ ID NO:6), wherein X^{Z} is R or Q.

3. The promolecule of claim 2, wherein Z comprises the amino acid sequence
(SEQ ID NO: 55)
SFLLRNPNDKYEPFW.

4. The promolecule of claim 1, wherein Z comprises the amino acid sequence
(SEQ ID NO: 56)
SFLLQDPNDQYEPFW.

5. The promolecule of claim 4, wherein Z comprises the amino acid sequence
(SEQ ID NO: 7)
QDPNDQYEPF.

6. The promolecule of claim 1, wherein portion Z comprises a covalently linked water soluble polymer.

7. The promolecule of any of claims 1 to 6, wherein A comprises a protein from the Temporin family.

8. The promolecule of claim 7, wherein A comprises the amino acid sequence X^{a}X^{b}X^{c}X^{d}X^{e}X^{f}Y^{a}X^{g}X^{h}Y^{b}Y*XⁱX^{j}, where X^{a}, X^{b}, X^{c}, X^{d}, X^{e}, X^{f}, X^{g}, X^{h}, Xⁱ, and X^{j} are hydrophobic amino acid residues, Y^{a} and Y^{b} are hydrophilic amino acid residues, and Y* is a charged amino acid residue.

9. The promolecule of claim 7, wherein A comprises the amino acid sequence FLP(X^{k})IASLL(X^{l})KLL (SEQ ID NO:8), wherein X^{k} is I or L and X^{l} is S or G.

10. The promolecule of claim 7, wherein A comprises the amino acid sequence
(SEQ ID NO: 1)
FVQWFSKFLGRIL
or
(SEQ ID NO: 2)
FVQWFSKFLGKLL.

11. The promolecule of claim 7, wherein A comprises the amino acid sequence
(SEQ ID NO: 3)
FVQWFSKFLGK.

12. The promolecule of any one of claims 1 to 11, wherein X² is a linker cleavable by granzyme A, B, H, K or M.

13. The promolecule of any one of claims 1 to 12, wherein X² is a linker cleavable by granzyme B.

14. The promolecule of claim 13, wherein X² comprises the amino acid sequence X^{m}XⁿPDX^{o}SX^{p}X^{q}, wherein:
X^{m} is V, L, or I;
Xⁿ is E;
X^{o} is F, S, or V;
X^{p} is T or Q; and
X^{q} is V.

15. The promolecule of claim 13, wherein X² comprises the amino acid sequence IEPDVSQV (SEQ ID NO:57), LTYDFWIQ (SEQ ID NO:65), PQVDLYDK (SEQ ID NO:66), VVQDKHEI (SEQ ID NO:67), VYADSSEW (SEQ ID NO:68), TMADSQES (SEQ ID NO:69), GHIDHMXX (SEQ ID NO:70), LEQDVWIA (SEQ ID NO:71), LDPDNFKR (SEQ ID NO:72), XXPDFYLG (SEQ ID NO:73), MGPDAFNL (SEQ ID NO:74), LKDDMGXX (SEQ ID NO:75), IWFDYTLK (SEQ ID NO:76), XIGDNVEW (SEQ ID NO:77), XXXDQVNL (SEQ ID NO:78), PQADQWXX (SEQ ID NO:79), PSVDMXXX (SEQ ID NO:80), XNVDWTAP (SEQ ID NO:81), YGYDLQTA (SEQ ID NO:82), HGFDEAHN (SEQ ID NO:83), HSHDSWKA (SEQ ID NO:84), KQDDLMSE (SEQ ID NO:85), SFGDIMEM (SEQ ID NO:86), VNDDVKXX (SEQ ID NO:87), XXXDKQFT (SEQ ID NO:88), or NDVDGGXX (SEQ ID NO:89), where X is any amino acid.

16. The promolecule of any one of claims 1 to 12, wherein X² is a linker cleavable by granzyme K.

17. The promolecule of claim 16, wherein X² comprises the amino acid sequence X^{r}X^{s}FRSX^{t}X^{u}X^{v}, wherein:
X^{r} is E or W;
X^{s} is F, Y, or A;
X^{t} is F, R, or I;
X^{u} is Y, P, or T; and
X^{v} is W or H.

18. The promolecule of claim 17, wherein X² comprises the amino acid sequence
(SEQ ID NO: 58)
WAFRSRYH.

19. The promolecule of any one of claims 1 to 18, wherein one or more of X^{1a}, X^{1b}, A, or Z comprises a D-amino acid.

20. The promolecule of any one of claims 1 to 19, wherein X^{1a} is present and comprises a nucleophilic moiety.

21. The promolecule of any one of claims 1 to 20, wherein X^{1b} is present and comprises a nucleophilic moiety.

22. The promolecule of claim 20 or claim 21, wherein the nucleophilic moiety of X^{1a} or X^{1b} comprises a thiol functional group.

23. The promolecule of claim 20 or claim 21, wherein X^{1a} or X^{1b} comprises an amino acid residue comprising the nucleophilic moiety.

24. The promolecule of claim 23, wherein the amino acid residue is a cysteine residue.

25. The promolecule of claim 23, wherein the amino acid residue is a lysine residue.

26. The promolecule of claim 20 or claim 21, wherein X^{1a} or X^{1b} comprises a cargo moiety covalently attached to the nucleophilic moiety.

27. The promolecule of claim 26, wherein the cargo moiety is a detectable moiety.

28. The promolecule of claim 27, wherein the detectable moiety is a metal chelating moiety.

29. The promolecule of claim 28, wherein the metal chelating moiety is 1,4,7,10-tetraazacyclododecane-1,4,7,10-tetraacetic acid (DOTA).

30. The promolecule of claim 29, wherein the metal chelating moiety is bound to a radioisotope.

31. The promolecule of claim 27, wherein the detectable moiety comprises a radioisotope.

32. The promolecule of claim 30 or claim 31, wherein the radioisotope is Actinium-225, Astatine-211, Bismuth-212, Bismuth-213, Bromine-76, Bromine-77, Calcium-47, Carbon-11, Carbon-14, Chromium-51, Cobalt-57, Cobalt-58, Copper-64, Erbium-169, Fluorine-18, Gallium-67, Gallium-68, Hydrogen-3, Indium-111, Iodine-123, Iodine-125, Iodine-131, Iron-59, Krypton-81m, Lead-212, Lutetium-177, Nitrogen-13, Oxygen-15, Phosphorus-32, Radium-223, Radium-224, Samarium-153, Selenium-75, Sodium-22, Sodium-24, Strontium-89, Technetium-99m, Thallium-201, Thorium-226, Thorium-227, Xenon-133, or Yttrium-9.

33. The promolecule of claim 32, wherein the radioisotope is Copper-64.

34. The promolecule of any one of claims 27 to 33, wherein the detectable moiety is detectable by positron emission tomography (PET).

35. The promolecule of claim 27, wherein the detectable moiety comprises a fluorescent moiety.

36. The promolecule of claim 35, wherein the fluorescent moiety is a water soluble fluorescent dye.

37. The promolecule of claim 36, wherein the water soluble fluorescent dye is a cyanine dye.

38. The promolecule of claim 37, wherein the cyanine dye is Cy7.

39. The promolecule of any one of claims 1 to 38, wherein the promolecule does not inhibit activity of the granzyme.

40. A nucleic acid encoding the promolecule of any one of claims 1 to 39.

41. An expression vector comprising the nucleic acid of claim 40.

42. A cell comprising the nucleic acid of claim 40 or the expression vector of claim 41.

43. A composition comprising:
the promolecule of any one of claims 1 to 39 present in a liquid medium.

44. A composition comprising:
the promolecule of any one of claims 1 to 39; and
a pharmaceutically acceptable carrier.

45. The composition of claim 43 or claim 44, wherein the promolecule comprises a detectable moiety as defined in any one of claims 28 to 38.

46. A kit comprising:
the composition of claim 45; and
instructions for using the composition to detect granzyme activity in vitro, in vivo or ex vivo.

47. A method of detectably labeling a phospholipid bilayer of a cell in the presence of granzyme activity, the method comprising:
contacting the promolecule of any one of claims 27 to 39 with granzyme contributing to the granzyme activity,
wherein the cleavable linker of the promolecule is cleaved by the granzyme to release a cleavage product comprising a detectable moiety and a membrane interacting polypeptide portion, such that the membrane interacting polypeptide portion interacts with the phospholipid bilayer of the cell and detectably labels the phospholipid bilayer of the cell in the presence of granzyme activity.

48. The method of claim 47, wherein the contacting is in vitro, in vivo, or ex vivo.

49. A method for assessing granzyme activity in a cellular sample, the method comprising:
contacting the sample with the promolecule of any one of claims 27 to 39, wherein in the presence of granzyme activity, the promolecule is cleaved to release a cleavage product comprising the detectable moiety and the membrane interacting polypeptide portion, and wherein the cleavage product interacts with a phospholipid bilayer of a cell in the presence of the granzyme activity; and
assessing for the presence or absence of the detectable moiety of the cleavage product,
wherein the presence of the detectable moiety indicates granzyme activity in the cellular sample.

50. A method for assessing granzyme activity in a subject, the method comprising:
administering the promolecule of any one of claims 27 to 39 to the subject, wherein at sites of granzyme activity in the subject, the promolecule is cleaved by a granzyme contributing to the granzyme activity to release cleavage products comprising the detectable moiety and the membrane interacting polypeptide portion, and wherein the cleavage products interact with the phospholipid bilayers of cells at the sites of granzyme activity in the subject; and
assessing for the presence or absence of cells labeled with the cleavage products,
wherein the presence of cells labeled with the cleavage products indicates granzyme activity in a subject.

51. A method of assessing for activation of immune cells in a subject, wherein the immune cells secrete granzyme upon activation in the subject, the method comprising:
administering the promolecule of any one of claims 27 to 39 to the subject, wherein at sites of activated immune cell-secreted granzyme in the subject, the promolecule is cleaved by the activated immune cell-secreted granzyme to release cleavage products comprising the detectable moiety and the membrane interacting polypeptide portion, and wherein the cleavage products interact with the phospholipid bilayers of cells at the sites of activated immune cell-secreted granzyme in the subject; and
assessing for the presence or absence of cells labeled with the cleavage products,
wherein the presence of cells labeled with the cleavage products indicates activation of the immune cells in the subject.

52. The method of claim 51, wherein the immune cells comprise cytotoxic T lymphocytes (CTLs).

53. The method of claim 51 or claim 52, wherein the immune cells comprise natural killer (NK) cells.

54. The method of any one of claims 51 to 53, wherein the immune cell-secreted granzyme is granzyme B.

55. The method of any one of claims 51 to 53, wherein the immune cell-secreted granzyme is granzyme K.

56. The method of any one of claims 51 to 55, comprising assessing for immune cell activation in the subject at the organismal level.

57. The method of any one of claims 51 to 56, comprising assessing for immune cell activation within a target region of the subject.

58. The method of any one of claims 51 to 57, wherein assessing for immune cell activation comprises distinguishing locations of immune cell activation in the subject.

59. The method of any one of claims 51 to 58, wherein assessing for immune cell activation occurs 4 hours or more following administration of the promolecule to the subject.

60. The method of any one of claims 51 to 59, wherein assessing for immune cell activation comprises assessing for immune cell activation at multiple time points.

61. The method of any one of claims 51 to 60, wherein the subject is undergoing an immunomodulatory therapy.

62. The method of claim 61, wherein the method comprises monitoring the progress of the immunomodulatory therapy by assessing for the presence or absence of cells labeled with cleavage products at the site of treatment, wherein the presence of cells labeled with the cleavage products indicates activation of the immune cells in the subject at the site of treatment.

63. The method of claim 61 or 62, wherein the immunomodulatory therapy comprises a cell-based therapy, stimulator of interferon genes (STING) pathway modulation, immune checkpoint inhibition, chemotherapy, ionizing radiation, or any combination thereof.

64. The method of claim 63, wherein the immunomodulatory therapy comprises a cell-based therapy.

65. The method of claim 64, wherein the cell-based therapy comprises a chimeric antigen receptor T-cell (CAR T) therapy.

66. The method of claim 64, wherein the cell-based therapy comprises a chimeric antigen receptor natural killer cell (CAR NK cell) therapy.

67. The method of claim 64, wherein the cell-based therapy comprises administration of T cells comprising an engineered T-cell receptor (TCR).

68. The method of any one of claims 61 to 67, wherein the immunomodulatory therapy is for treating cancer in the subject, and wherein assessing for immune cell activation further comprises assessing a tumor in the subject for responsiveness to the immunomodulatory therapy.

69. The method of any one of claims 51 to 68, wherein assessing for immune cell activation comprises assessing for the presence or absence of T-cell exhaustion.

70. The method of any one of claims 51 to 69, wherein assessing for immune cell activation comprises assessing for an immune response in the subject.

71. The method of claim 70, wherein assessing for immune cell activation comprises assessing for an immune response to an infection in the subject.

72. The method of claim 71, wherein assessing for immune cell activation comprises assessing for an immune response to a viral infection in the subject.

73. The method of claim 72, wherein the viral infection causes pneumonia.

74. The method of claim 71, wherein assessing for immune cell activation comprises assessing for an immune response to a bacterial infection in the subject.

75. The method of claim 74, wherein the bacterial infection is an infection of Escherichia coli (E. coli).

76. The method of claim 74, wherein the bacterial infection is an infection of Staphylococcus aureus (S. aureus).

77. The method of claim 74, wherein the bacterial infection is an infection of Pseudomonas aeruginosa (P. aeruginosa).

78. The method of claim 74, wherein the bacterial infection is an infection of Klebsiella pneumoniae (K. pneumoniae).

79. The method of claim 70, wherein the presence of systemic immune cell activation in normal tissues indicates that the subject is at risk for developing immune related adverse effects.

80. The method of any one of claims 50 to 79, wherein the subject is a human.

81. The method of any one of claims 47 to 80, wherein the detectable moiety comprises a radioisotope.

82. The method of any one of claims 47 to 81, wherein the assessing comprises detecting the detectable moiety by PET.

83. A method of making a promolecule useful in delivery of a cargo moiety to a phospholipid bilayer, the method comprising:
synthesizing the promolecule of any one of claims 1 to 39, wherein X^{1a} is present; and
attaching a cargo moiety to the nucleophilic moiety of X^{1a},
wherein a promolecule useful in delivery of a cargo moiety to a phospholipid bilayer is produced.

84. The method of claim 83, wherein said synthesizing comprises culturing a recombinant host cell comprising an expression construct encoding the promolecule.

85. The method of claim 83, wherein said synthesizing is by chemical synthesis.
